(19) United States
(12) Patent Application Publication
(10) Pub. No.: US 2026/0272982 A1
(43) Pub. Date: Sep. 17, 2026

(54) MANUFACTURING OF THERAPEUTIC CELLS

(71) Applicant: CYTONUS THERAPEUTICS, INC., Carlsbad, CA (US)

(72) Inventors: Remo MOOMIAIE, Carlsbad, CA (US); Yuval ZUR, Carlsbad, CA (US)

(21) Appl. No.: 19/168,626

(22) PCT Filed: Apr. 11, 2024
(86) PCT No.: PCT/US2024/024070

§ 371 Date: Sep. 24, 2025

Related U.S. Application Data
(60) Provisional application No. 63/495,697, filed on Apr. 12, 2023.

(51) Int. Cl. A61K 35/17; A61K 35/28; A61K 38/00; A61K 40/15; A61K 40/42; A61K 45/06; C12N 5/00; C12N 5/0775; C12N 5/0783; C12N 15/11

(52) U.S. Cl. CPC A61K 35/17; A61K 35/28; A61K 38/00; A61K 40/15; A61K 40/42; A61K 45/06; C12N 5/0081; C12N 5/0646; C12N 5/0662; C12N 5/562; C12N 15/11; C12N 2510/00

(57) ABSTRACT

Described herein are methods for obtaining enucleated cells from nucleated cells. Also, described herein are methods for cell processing, including providing a composition containing nucleated cells and enucleating at least a portion of the nucleated cells to produce an enucleated cell fraction.

### CROSS-REFERENCE

This application claims the benefit of U.S. Provisional Application Ser. No. 63/495,697 filed on Apr. 12, 2023, the entirety of which is hereby incorporated by reference herein.

### BACKGROUND

Current cell-based therapies are prone to unwanted side effects, such as oncogenicity, limited engineering capability, and deleterious immune response. Additionally, generating sufficient quantity of cell-based therapies remains an ongoing challenge.

### SUMMARY

Accordingly, there remains a need for large-scale manufacturing of cell-based therapies. Described herein, in some aspects, is a method for obtaining a population of enucleated cells, the method comprising: contacting a plurality of cells with a particle density gradient generated by fixed angle centrifugation to produce the population of enucleated cells, where the population of enucleated cells accumulates at a density range in the particle density gradient. In some embodiments, the method further comprises contacting the plurality of cells with a toxin, wherein the toxin inhibits cytoskeletal formation of the plurality of cells. In some embodiments, the method further comprises removing the population of enucleated cells from the particle density gradient. In some embodiments, the toxin comprises a mycotoxin. In some embodiments, the mycotoxin comprises a cytochalasin. In some embodiments, the cytochalasin comprises a cytochalasin B. In some embodiments, the population of cells, after enucleation, comprises a decreased density compared to the plurality of cells having a nucleus. In some embodiments, the particle density gradient, prior to the fixed angle centrifugation, comprises a plurality of particle solutions comprising different particle concentrations. In some embodiments, the fixed angle centrifugation comprises centrifugating the plurality of cells in the particle density gradient at a 23-degree angle, at a 24-degree angle, at a 25-degree angle, at a 45-degree angle, at a 90-degree angle, or at a 180-degree angle. In some embodiments, the fixed angle centrifugation comprises centrifugation at about 80,000 RCF, at about 83,000 RCF, at about 85,000 RCF, at about 87,000 RCF, at about 90,000 RCF, at about 93,000 RCF, at about 95,000 RCF, at about 97,000 RCF, at about 100,000 RCF, at about 103,000 RCF, at about 105,000 RCF, at about 107,000 RCF, at about 110,000 RCF, at about 113,000 RCF, at about 115,000 RCF, at about 117,000 RCF, at about 120,000 RCF, at about 123,000 RCF, at about 125,000 RCF, at about 127,000 RCF, at about 130,000 RCF, at about 133,000 RCF, at about 135,000 RCF, or at about 137,000 RCF, In some embodiments, the fixed angle centrifugation comprises centrifugation at about 85,000 RCF. In some embodiments, the fixed angle centrifugation comprises centrifugation at about 127,000 RCF. In some embodiments, the fixed angle centrifugation comprises centrifugation at an r average of about 85,000 RCF. In some embodiments, the fixed angle centrifugation comprises centrifugation at an r max of about 127,000 RCF. In some embodiments, the fixed angle centrifugation comprises acceleration comprising about 5.2 min to reach RCF for centrifugation. In some embodiments, the fixed angle centrifugation comprises deceleration comprising about 4.0 min to reach a stoppage of the fixed angle centrifugation. In some embodiments, the fixed angle centrifugation comprises centrifugation for about 60 minutes. In some embodiments, the particle density gradient comprises a plurality of particle solutions. In some embodiments, the plurality of particle solutions comprises at least three particle solutions. In some embodiments, the particle density gradient comprises a protein density gradient. In some embodiments, the protein density gradient comprises a serum albumin gradient. In some embodiments, the particle density gradient comprises a polysaccharide density gradient. In some embodiments, the polysaccharide density gradient comprises a plurality of polysaccharide solutions. In some embodiments, the plurality of polysaccharide solutions comprises: a 12.5% polysaccharide solution, a 15% polysaccharide solution, a 16% polysaccharide solution, a 17% polysaccharide solution, a 25% polysaccharide solution, or any combination thereof. In some embodiments, the population of enucleated cells accumulates at a density range of about 15% polysaccharide. In some embodiments, the polysaccharide density gradient comprises a Ficoll density gradient. In some embodiments, the plurality of cells comprises a heterologous polynucleotide. In some embodiments, the method further comprises cryopreserving the population of cells. In some embodiments, the fixed angle centrifugation comprises ultracentrifugation. In some embodiments, the plurality of cells comprises stem cells. In some embodiments, the stem cells comprise induced pluripotent stem cells (iPSCs), adult stem cells, mesenchymal stromal cells, embryonic stem cells, fibroblasts, immortalized cells from a cell line, or any combination thereof. In some embodiments, the stem cells comprise the mesenchymal stromal cells. In some embodiments, the plurality of cells comprises immune cells. In some embodiments, the immune cells comprise lymphocytes or natural killer cells. In some embodiments, the plurality of cells comprises one or more intracellular organelles for synthesis or secretion of an exogenous polypeptide, wherein the one or more intracellular organelles is retained in the population of enucleated cells, and wherein the one or more intracellular organelles synthesizes or secretes the exogenous polypeptide in an absence of a nucleus. In some embodiments, the plurality of cells comprises a heterologous polynucleotide encoding the exogenous polypeptide. In some embodiments, the population of enucleated cells comprises a heterologous polynucleotide encoding the exogenous polypeptide, wherein the heterologous polynucleotide is introduced into the population of enucleated cells and the one or more intracellular organelles synthesizes or secretes the exogenous polypeptide in an absence of a nucleus. In some embodiments, the exogenous polypeptide comprises a therapeutic agent. In some embodiments, the population of enucleated cells comprise at least one targeting moiety. In some embodiments, the population of enucleated cells comprise at least one fusogenic moiety. In some embodiments, the population of enucleated cells comprise at least one immune evasion moiety. In some embodiments, an enucleated cell of the population of enucleated cells has a diameter comprising less than or equal to about 10%, about 20%, about 30%, about 40%, about 50%, about 60%, about 70%, about 80%, about 90%, about 95%, or about 99% of an average diameter of the nucleated cells. In some embodiments, an enucleated cell of the population of enucleated cells has a diameter comprising more than or equal to about 5 μm, about 10 μm, about 20 μm, about 30 μm, about 40 μm, about 50 μm, about 60 μm, about 70 μm, about 80 μm, or about 90 μm. In some embodiments, the diameter comprises about 8 μm.

Described herein, in some aspects, is a pharmaceutical composition comprising: the population of enucleated cells produced by a method described herein; and a pharmaceutically acceptable: excipient, carrier, or diluent. In some embodiments, the pharmaceutical composition is in a unit dose form. In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or any combination thereof, to a subject. In some embodiments, the pharmaceutical composition is formulated for administering intravenously. In some embodiments, the pharmaceutical composition further comprises at least one additional active agent. In some embodiments, the at least one additional active agent comprises a cytokine, a growth factor, a hormone, an enzyme, a small molecule, a compound, or any combination thereof.

Described herein, in some aspects, is a kit comprising: the population of cells produced by a method described herein or a pharmaceutical composition described herein; and a container.

Described herein, in some aspects, is a kit comprising instructions for enucleating the plurality of cells according to a method described herein to produce the population of enucleated cells; and one or more components of the polysaccharide density gradient.

Described herein, in some aspects, is a method of treating a disease or condition of a subject comprising: administering to the subject a therapeutically effective amount of a population of enucleated cells produced by a method described herein or a pharmaceutical composition described herein.

Described herein, in some aspects, is a method of treating cancer in a subject, the method comprising: administering to the subject with cancer a therapeutically effective amount of a population of enucleated cells produced by a method described herein or a pharmaceutical composition described herein.

Described herein, in some aspects, is a method of treating a lung disease in a subject, the method comprising: administering to the subject with the lung disease a therapeutically effective amount of a population of enucleated cells produced by a method described herein or a pharmaceutical composition described herein.

Described herein, in some aspects, is a method for obtaining a population of cells from a plurality of cells, comprising: contacting the plurality of cells with a polysaccharide density gradient generated by fixed angle centrifugation, wherein the population of cells accumulates at a density range in the polysaccharide density gradient. Also described herein, in some aspects, is a method for enucleating a population of cells from a plurality of cells, comprising: contacting the plurality of cells with a toxin, said toxin inhibits cytoskeletal formation of the plurality of cells; contacting the plurality of cells with a polysaccharide density gradient generated by fixed angle centrifugation, wherein the population of cells are enucleated by the fixed angle centrifugation and accumulates at a density range in the polysaccharide density gradient. In some embodiments, the method further comprises removing the population of cells from the polysaccharide density gradient. In some embodiments, the toxin comprises a mycotoxin. In some embodiments, the mycotoxin comprises a cytochalasin. In some embodiments, the cytochalasin comprises a cytochalasin B. In some embodiments, the population of cells, after enucleation, comprises a decreased density compared to the plurality of cells having nucleus. In some embodiments, the polysaccharide density gradient, prior to the fixed angle centrifugation, comprises a plurality of polysaccharide solutions comprising different polysaccharide concentrations. In some embodiments, the fixed angle centrifugation comprises centrifugating the plurality of cells in the polysaccharide density gradient at a 23-degree angle, at a 24-degree angle, at a 25-degree angle, at a 45-degree angle, at a 90-degree angle, or at a 180-degree angle. In some embodiments, the fixed angle centrifugation comprises centrifugation at about 126000 RCF. In some embodiments, the fixed angle centrifugation comprises centrifugation at an r average of about 85,000 RCF. In some embodiments, the fixed angle centrifugation comprises centrifugation at an r max of about 127,000 RCF. In some embodiments, the fixed angle centrifugation comprises acceleration comprising about 5.2 min to reach RCF for centrifugation. In some embodiments, the fixed angle centrifugation comprises deceleration comprising about 4.0 min to reach a stoppage of the fixed angle centrifugation. In some embodiments, the fixed angle centrifugation comprises centrifugation for about 60 minutes. In some embodiments, the polysaccharide density gradient comprises a plurality of polysaccharide solutions. In some embodiments, the plurality of polysaccharide solutions comprises at least three polysaccharide solutions. In some embodiments, the plurality of polysaccharide solutions comprises: a 12.5% polysaccharide solution, a 15% polysaccharide solution, a 16% polysaccharide solution, a 17% polysaccharide solution, a 25% polysaccharide solution, or a combination thereof. In some embodiments, the population of cells accumulates at the density range of about 15% polysaccharide. In some embodiments, the polysaccharide density gradient comprises a Ficoll density gradient. In some embodiments, the plurality of cells comprises a heterologous polynucleotide. In some embodiments, the method further comprises cryopreserving the population of cells. In some embodiments, the method further comprises thawing the population of cells, wherein, following the thawing, an enucleated cell of the population of cells is as viable as an otherwise comparable enucleated cell that is not cryopreserved. In some embodiments, the population of cells comprises stem cells. In some embodiments, the stem cells comprise induced pluripotent stem cells (iPSCs), adult stem cells, mesenchymal stromal cells, embryonic stem cells, fibroblasts, or immortalized cells from a cell line, or a combination thereof. In some embodiments, the stem cells comprise the mesenchymal stromal cells. In some embodiments, the population of cells comprises immune cells. In some embodiments, the immune cells comprise lymphocytes or natural killer cells. In some embodiments, the population of cells comprise one or more intracellular organelles for synthesis or secretion of an exogenous polypeptide in absence of a nucleus. In some embodiments, the exogenous polypeptide is encoded by the heterologous polynucleotide. In some embodiments, the exogenous polypeptide comprises a therapeutic agent. In some embodiments, the population of cells comprise at least one targeting moiety. In some embodiments, the population of cells comprise at least one fusogenic moiety. In some embodiments, the population of cells comprise at least one immune evasion moiety. In some embodiments, an enucleated cell of the population of cells has a diameter comprising less than or equal to about 10%, about 20%, about 30%, about 40%, about 50%, about 60%, about 70%, about 80%, about 90%, about 95%, or about 99% of an average diameter of the nucleated cells. In some embodiments, an enucleated cell of the population of cells has a diameter comprising more than or equal to about 5 μm, about 10 μm, about 20 μm, about 30 μm, about 40 μm, about 50 μm, about 60 μm, about 70 μm, about 80 μm, or about 90 μm. In some embodiments, the diameter comprises about 8 μm.

Described herein, in some aspects, is a pharmaceutical composition comprising: the population of cells described herein; and a pharmaceutically acceptable: excipient, carrier, or diluent. In some embodiments, the pharmaceutical composition is in a unit dose form. In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof, to a subject. In some embodiments, the pharmaceutical composition is formulated for administering intravenously. In some embodiments, pharmaceutical composition further comprises at least one additional active agent. In some embodiments, the at least one additional active agent comprises a cytokine, a growth factor, a hormone, an enzyme, a small molecule, a compound, or a combination thereof.

Described herein, in some aspects, is a kit comprising: the population of cells described herein or the pharmaceutical composition described herein; and a container.

Described herein, in some aspects, is a method of treating a disease or condition of a subject comprising: administering to the subject a therapeutically effective amount of the population of cells described herein or the pharmaceutical composition described herein.

Described herein, in some aspects, is a method of treating cancer in a subject, the method comprising: administering to the subject with cancer a therapeutically effective amount of the population of cells described herein or the pharmaceutical composition described herein.

Described herein, in some aspects, is a method of treating a lung disease in a subject, the method comprising: administering to the subject with the lung disease a therapeutically effective amount of the population of cells described herein or the pharmaceutical composition described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Some novel features of the methods and compositions disclosed herein are set forth in the present disclosure. A better understanding of the features and advantages of the methods and compositions disclosed herein will be obtained by reference to the following detailed description that sets forth illustrative embodiments, in which the principles of the disclosed compositions and methods are utilized, and the accompanying drawings of which:

FIG. 1 illustrates a flow chart showing non-limiting steps of a process for composition or pharmaceutical composition of enucleated cells for delivery of therapeutics, according to an embodiment of the present disclosure.

FIG. 2 illustrates a timeline for production of the enucleated cells for the delivery of the single-domain antibody according to various embodiments, as compared to a typical biological drug development timeline.

FIG. 3A illustrates enucleation of the cells in a fixed-angle centrifuge with three Ficoll gradients. The three Ficoll gradients used are 12.5%, 15%, and 17% with the enucleated cells located in the 15% gradient following enucleation by fixed angle centrifugation.

FIG. 3B illustrates images of the enucleated cells isolated from the 15% Ficoll gradient following enucleation in a fixed angle centrifuge. The image in the center shows the enucleated cells imaged with Bright-field microscopy. The image on the right shows the DAPI-stained enucleated cells imaged with fluorescent microscopy, where the enucleated cells did not exhibit DAPI-staining.

### DETAILED DESCRIPTION

Existing techniques of displacing nuclei in adherent cells, or enucleation, using centrifugal force have drawbacks rending them inoperable for large scale of an enucleated cell platform for biomedical applications. Such drawbacks include, without limitation, small capacities of swinging buckets, ranging from a few milliliters to a few hundred milliliters, and long pathlengths for overall pelleting time, resulting in small-scale production with low efficiency. These drawbacks greatly restrict the number of cells processable at once, making it difficult to achieve optimal cell density in each centrifugation bucket for large scale manufacturing.

To overcome these drawbacks, the present application discloses methods for cell processing comprising enucleating cells using fixed angle centrifugation, including continuous flow centrifugation. Due to their simple and efficient tube spacing, fixed-angle rotors can hold a greater quantity of tubes compared to its swinging-bucket counterpart, Also, as a result of the rigid design of the metal alloy material, fixed rotors can withstand much higher gravitational forces, and thus, shorter centrifugation times. In a fixed angle centrifugation, including continuous flow centrifugation, large volumes of material can be centrifuged at high centrifugal forces without the tedium of filling and decanting a lot of centrifuge tubes, or frequently starting and stopping the rotor. This combination of high centrifugal force, short centrifugation, and high throughput makes fixed angle centrifugation processing useful for the large-scale manufacturing of enucleated cells for biomedical application, such as pelleting bacteria and cells to isopycnic separation of macromolecules.

In addition, quality control of enucleated cell platforms for biomedical applications is a challenge, which become magnified by large scale manufacturing. Some of the many benefits of the enucleated cells disclosed herein are attributed to an absence of a nucleus, such as unwanted gene transfer in vivo, limited lifespan in vivo, and so forth. However, existing large scale manufacturing techniques result in a portion of nucleated parent cells in the resulting therapeutic composition, obviating the benefits of the enucleated cell platform.

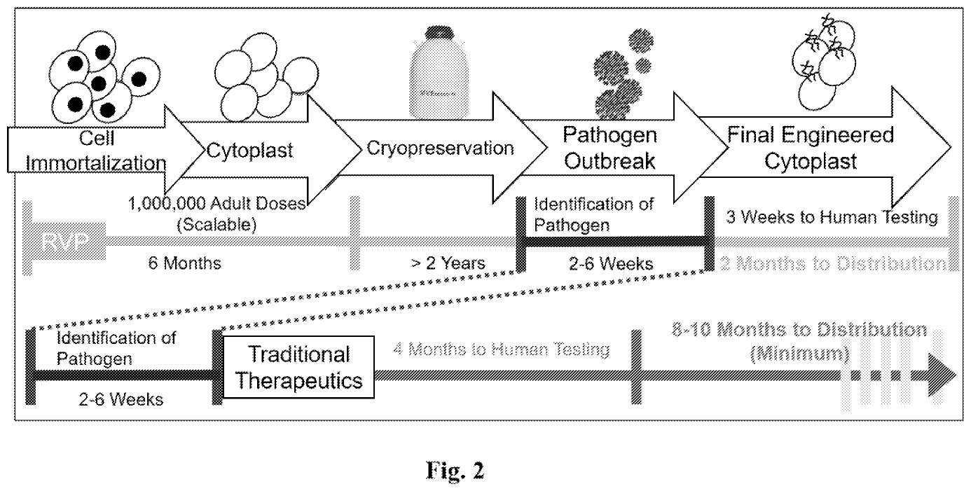

In addition to the advances in manufacturing scalability and quality control, the enucleated cell platform, itself, described herein possess certain advantages over existing cell-based therapeutic platforms that make it uniquely suitable for large scale use as therapeutic compositions. The enucleated cells described herein may be found in United States Patent Application No. 10,927,349, which is hereby incorporated by reference in its entirety. In addition, additional utility and advantages of the enucleated cells disclosed herein are discussed in U.S. patent application Ser. No. 18/176,259, filed Feb. 28, 2023; U.S. patent application Ser. No. 17/885,867, filed Aug. 11, 2022; and U.S. patent application Ser. No. 18/190,838, filed Mar. 27, 2023, each of which is hereby incorporated by reference in its entirety. For example, there are certain therapeutic applications of cellular delivery platforms, such as in response to a pathogen outbreak, for which existing manufacturing timelines can limit scalability and speed necessary to address a public health emergency due to a pathogen outbreak. Existing therapeutic cellular therapies requiring extensive engineering take on the order of 12 months to develop at minimum. Whereas the enucleated cells disclosed herein can be extensively engineered before and after enucleation (e.g., with targeting moieties specific to target tissue, immune-system evading moieties to reduce phagocytosis in vivo, etc.), and then stored by suitable means disclosed here (e.g., lyophilization, cryohibernation, and cryopreservation) for extended periods of time without sacrificing viability once revived. When a new pathogen or new strain of a known pathogen is identified, the biological activity of the enucleated cells (already engineered to express the appropriate targeting moieties, immune-system evading moieties, immune activators, etc.) can be restored (e.g., rehydration, thawing, etc.) and further engineered to express or carry a therapeutic agent for the prophylaxis or treatment of an infection by that recently discovered pathogen or strain. These benefits can be seen in FIG. 2, which illustrates that the process of manufacturing the enucleated cells of the present disclosure is roughly 2 months, as compared with suitable timelines, which is 12 months or longer.

Existing red blood cell or platelet therapeutic platforms are enucleated by erythropoiesis in which the blood cell is terminally differenced and intracellular organelles and ribosomes are eliminated, some of which are responsible for protein synthesis and secretion. Thus, the resulting red blood cell or platelet loses the cell-like functionality (e.g., protein expression, secretion, cell motility, chemokine sensing, homing capabilities, etc.) after enucleation by erythropoiesis that may be important for therapeutic applications, such as producing, delivering or secreting a therapeutic agent in vivo. By contrast, the enucleated cells described herein retain one or more intracellular organelles after enucleation that are endogenous to the parent cell. In some embodiments, all of the one or more intracellular organelles are retained. In some embodiments, fewer than all of the one or more intracellular organelles are retained. In some embodiments, the Golgi apparatus and/or the endoplasmic reticulum are retained, which are involved in protein synthesis and secretion. Retention of the one or more intracellular organelles at least partially enables the enucleated cells to synthesize or release the biomolecule disclosed herein (e.g., single-domain antibody, or portion thereof, targeting moiety, immune-evading moiety, etc.) in the absence of the nucleus. In some embodiments, the population of cells (e.g., enucleated cells) comprise one or more intracellular organelles. In some embodiments, the one or more intracellular organelles can synthesize and/or secrete an exogeneous polypeptide in the absence of a nucleus.

The enucleated cells disclosed herein may be derived from virtually any nucleated cell (referred to herein as “parent” cell). In some embodiments, the parent cell is an immune cell. In some embodiments, the immune cell is a neutrophil, eosinophil, basophil, mast cell, monocyte, macrophage, dendritic cell, natural killer cell, or lymphocyte (B cells and T cells). In some embodiments, the parent cell is a stem cell. In some embodiments, the parent cell is an adult stem cell. In some embodiments, the parent cell is a mesenchymal stromal cell (MSC). In some embodiments, the enucleated cell is derived from an inducible pluripotent stem cell (iPSC). In some embodiments, the parent cell is not an erythrocyte. In some embodiments, the parent cell is not an erythroid precursor cell. In some embodiments, the parent cell is not an endothelial cell. In some embodiments, the parent cell is not an endothelial precursor cell.

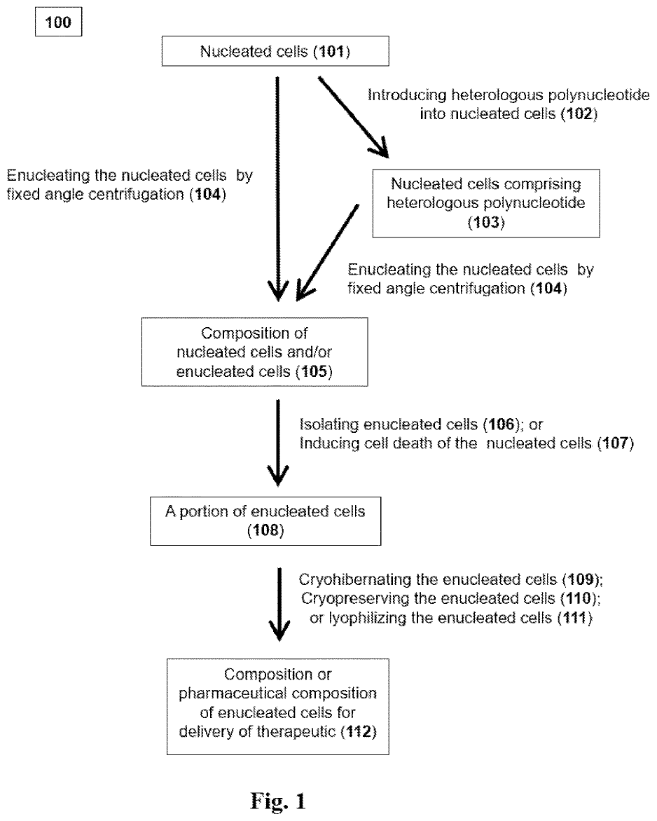

Described herein are methods for manufacturing enucleated cells in an increased quantity and purity, where the manufactured enucleated cells can be formulated into a composition or a pharmaceutical composition for treating a disease or condition in a subject in need thereof. FIG. 1 illustrates a non-limiting example of the manufacturing of the enucleated cells described herein (100). Nucleated cells (101) can be isolated from the subject and cultured in vitro for clonal expansion. In some embodiments, the nucleated cells (101) can also be immortalized or derived from a cell line. In some embodiments, the nucleated cells can be engineered (103) to comprise a heterologous polynucleotide (102). The nucleated cells can then be enucleated by fixed angle centrifugation, including continuous flow centrifugation, (104). The use of fixed angle centrifugation, including continuous flow centrifugation, for enucleating cells presents an improvement over the currently available methods for enucleation, where the enucleation conducted via fixed angle centrifugation, including continuous flow centrifugation, increases the quantity (e.g., yield) or purity of the enucleated cells obtained from the nucleated cells. In some embodiments, the fixed angle centrifugation yields an increased number of the population of enucleated cells. In some embodiments, the fixed angle centrifugation yields an increased number of the population of enucleated cells by at least 5%, at least 10%, at least 20%, at least 25%, at least 50%, at least 75%, at least 100%, 150%, at least 200%, at least 250%, at least 300%, at least 400%, at least 500%, at least 600%, at least 750%, at least 800%, at least 900%, or at least 1,000%. In some embodiments, the fixed angle centrifugation, compared to swing bucket centrifugation, results in higher yield of the population of enucleated cells. In some embodiments, the fixed angle centrifugation, compared to swing bucket centrifugation, results in at least 5%, at least 10%, at least 20%, at least 25%, at least 50%, at least 75%, at least 100%, 150%, at least 200%, at least 250%, at least 300%, at least 400%, at least 500%, at least 600%, at least 750%, at least 800%, at least 900%, at least 1,000%, at least 2,000%, at least 3,000%, at least 4,000%, at least 5,000%, at least 6,000%, at least 7,000%, at least 8,000%, or more in yield of the population of the enucleated cells. After obtaining the composition (105) of the enucleated cells (which may have residual nucleated cells), the composition can be further purified for the enucleated cells by selecting for markers of the enucleated cells (106) to obtain a portion of enucleated cells (107). The portion of enucleated cells can be cryohibernated (108), cryopreserved (109), lyophilized (110), or a combination thereof and be formulated into a composition or a pharmaceutical composition for delivery of therapeutic for treating the disease or condition in the subject (111).

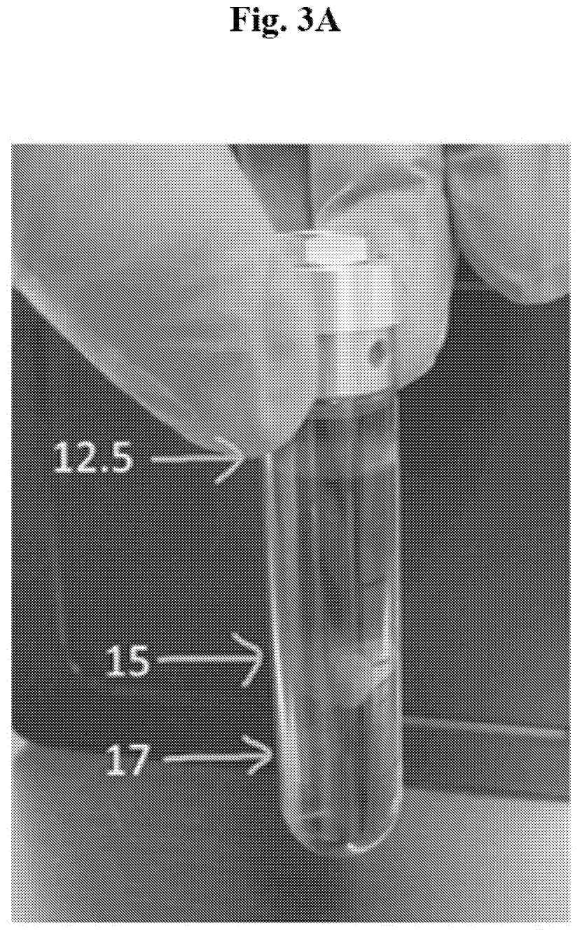

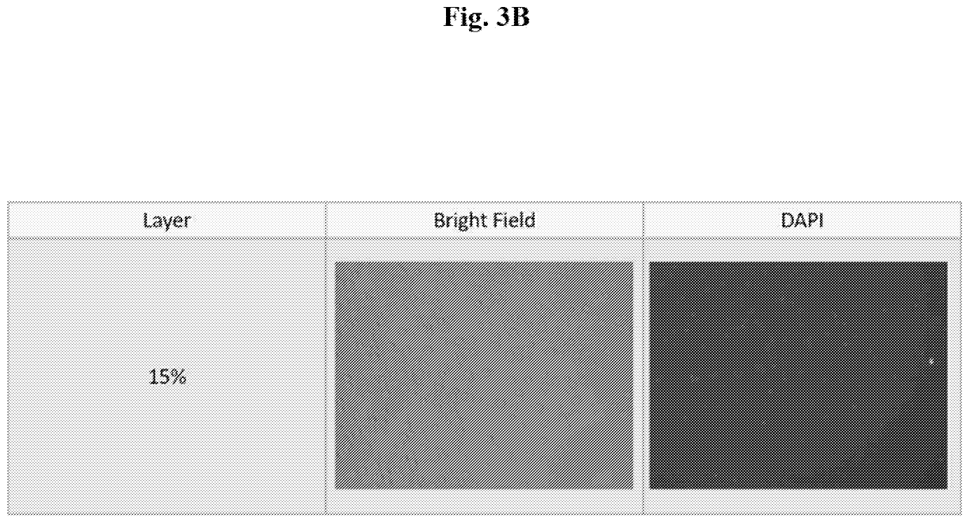

In some embodiments, the average enucleation yield is based on the total number of cells per loaded rotor. In some embodiments, average enucleation yield is about 40%, about 45%, about 50%, about 55%, about 60%, about 65%, about 70%, or about 75% of the total number of cells per loaded rotor. In some embodiments, the average enucleation efficiency is based on the average enucleation yield. In some embodiments, the average enucleation efficiency is about 80%, about 85%, about 90%, about 92%, about 94%, about 95%, about 96%, about 98% or about 99% based on the average enucleation yield. For example, after fixed angle centrifugation of cells, the fraction containing the enucleated cells is the enucleation yield and the number of actual enucleated cells in the fraction containing the enucleated cells is the enucleation efficiency. In some embodiments, the enucleation efficiency is determined by contacting the cells from the fraction containing the enucleated cells with a stain that stains nuclei. In some embodiments, the stain is a Hoechst stain. In some embodiments, the stain is a DAPI stain. For example. As shown in FIG. 3B, cells taken from a fraction of the tube comprising a 15% polysaccharide solution (Left) were examined under bright field (Center) and florescent (Right) microscopy. The bright field microscopy shows a lot of cells; however, once stained with DAPI and analyzed under florescent microscopy, there are fewer cells visible. In this experiment, the DAPI stains nuclei and the unstained/non-visualized cells under florescent microscopy have been successfully enucleated.

Described herein, in some aspects, is obtaining a population of enucleated cells from a plurality of cells, comprising contacting a plurality of cells with a particle density gradient generated by fixed angle centrifugation. In some embodiments, the particle density gradient comprises a polysaccharide density gradient. In some embodiments, the population of cells accumulates at a density range in the polysaccharide density gradient. In some embodiments, the method comprises contacting a plurality of cells with a toxin. In some embodiments, the toxin inhibits cytoskeletal formation of the plurality of cells. The toxin can be mycotoxin. The mycotoxin can comprise cytochalasin. The cytochalasin can comprise cytochalasin B. In some embodiments, the method comprises contacting the plurality of cells with a polysaccharide density gradient generated by a fixed angle centrifugation. In some embodiments, the population of cells are enucleated by the fixed angle centrifugation and accumulate at a density range in the polysaccharide density gradient. The population of cells can comprise a decreased density following enucleation as compared to the plurality of cells with a nucleus. In some embodiments, the method comprises removing the population of cells from the polysaccharide density gradient. In some embodiments, the method comprises contacting the population of cells with a polysaccharide density gradient. The polysaccharide density gradient can comprise a plurality of polysaccharide solutions comprising different polysaccharide concentrations prior to fixed angle centrifugation. In some embodiments, the method comprises subjecting the population of cells in the polysaccharide density to fixed angle centrifugation. In some embodiments, the fixed angle centrifugation comprises centrifugating the plurality of cells in the polysaccharide density gradient at about a 20-degree angle, about a 21-degree angle, about a 22-degree angle, about a 23-degree angle, about a 24-degree angle, about a 25-degree angle, about a 26-degree angle, about a 27-degree angle, about a 28-degree angle, about a 29-degree angle, about a 30-degree angle, about a 35-degree angle, about a 40-degree angle, about a 45-degree angle, about a 50-degree angle, about a 60-degree angle, about a 70-degree angle, about an 80-degree angle, about a 90-degree angle, about a 100-degree angle, about a 120-degree angle, about a 150-degree angle, or about a 180-degree angle. In some embodiments, the fixed angle centrifugation comprises centrifugating the plurality of cells in the polysaccharide density gradient at a 23-degree angle, at a 24-degree angle, at a 25-degree angle, at a 45-degree angle, at a 90-degree angle, or at a 180-degree angle. In some embodiments, the fixed angle centrifugation comprises centrifugation at about 120,000 RCF, about 122,000 RCF, about 124,000 RCF, about 126,000 RCF, about 127,000 RCF, about 128,000 RCF, about 130,000 RCF, about 132,000 RCF, about 134,000 RCF, or about 135,000 RCF. In some embodiments, the fixed angle centrifugation comprises centrifugation at about 127,000 RCF. In some embodiments, the fixed angle centrifugation comprises centrifugation at an r average of about 70,000 RCF, about 75,000 RCF, about 80,000 RCF, about 85,000 RCF, about 90,000 RCF, about 95,000 RCF, or about 100,000 RCF. In some embodiments, the fixed angle centrifugation comprises centrifugation at an r average of about 85,000 RCF. In some embodiments, the fixed angle centrifugation comprises centrifugation at an r max of about 120,000 RCF, about 122,000 RCF, about 124,000 RCF, about 126,000 RCF, about 127,000 RCF, about 128,000 RCF, about 130,000 RCF, about 132,000 RCF, about 134,000 RCF, or about 135,000 RCF. In some embodiments, the fixed angle centrifugation comprises centrifugation at an r max of about 127,000 RCF. In some embodiments, the fixed angle centrifugation comprises acceleration comprising about 3.0 minutes (min), about 3.2 min, about 3.4 min, about 3.5 min, about 3.6 min, about 3.8 min, about 4.0 min, about 4.2 min, about 4.4 min, about 4.5 min, about 4.6 min, about 4.8 min, about 5.0 min, about 5.2 min, about 5.4 min, about 5.5 min, about 5.6 min, about 5.8 min, about 6.0 min, about 6.2 min, about 6.4 min, about 6.5 min, about 6.6 min, about 6.8 min, or about 7.0 min to reach RCF for centrifugation. In some embodiments, the fixed angle centrifugation comprises acceleration comprising about 5.2 min to reach RCF for centrifugation.

In some embodiments, the fixed angle centrifugation comprises deceleration comprising about 2.0 minutes (min), about 2.2 min, about 2.4 min, about 2.5 min, about 2.6 min, about 2.8 min, about 3.0 minutes (min), about 3.2 min, about 3.4 min, about 3.5 min, about 3.6 min, about 3.8 min, about 4.0 min, about 4.2 min, about 4.4 min, about 4.5 min, about 4.6 min, about 4.8 min, about 5.0 min, about 5.2 min, about 5.4 min, about 5.5 min, about 5.6 min, about 5.8 min, or about 6.0 min to reach a stoppage of the fixed angle centrifugation. In some embodiments, the fixed angle centrifugation comprises deceleration comprising about 4.0 min to reach a stoppage of the fixed angle centrifugation. In some embodiments, the fixed angle centrifugation comprises centrifugation for about 30 minutes, about 40 minutes, about 45 minutes, about 50 minutes, about 55 minutes, about 60 minutes, about 65 minutes, about 70 minutes, about 75 minutes, about 80 minutes, or about 90 minutes. In some embodiments, the fixed angle centrifugation comprises centrifugation for about 60 minutes. In some embodiments, the method comprises subjecting the plurality of the cells to the fixed angle centrifugation for about 6 minutes. In some embodiments, the method comprises subjecting the plurality of the cells to the fixed angle centrifugation for about 5 minutes. In some embodiments, the method comprises subjecting the plurality of the cells to the fixed angle centrifugation for about 4 minutes.

In some embodiments, the method comprises subjecting the plurality of the cells to the fixed angle centrifugation comprising at a plurality of polysaccharide solutions. In some embodiments, the method comprises subjecting the plurality of the cells to the fixed angle centrifugation comprising at least two, at least three, at least four, at least five, at least six polysaccharide solutions. In some embodiments, the method comprises subjecting the plurality of the cells to the fixed angle centrifugation comprising at least three polysaccharide solutions. In some embodiments, the plurality of polysaccharide solutions comprises a 10% polysaccharide solution, a 10.5% polysaccharide solution, a 11% polysaccharide solution, a 11.5% polysaccharide solution, a 12% polysaccharide solution, a 12.5% polysaccharide solution, a 13% polysaccharide solution, a 13.5% polysaccharide solution, a 14% polysaccharide solution, a 14.5% polysaccharide solution, a 15% polysaccharide solution, a 15.5% polysaccharide solution, a 16% polysaccharide solution, a 16.5% polysaccharide solution, a 17% polysaccharide solution, a 17.5% polysaccharide solution, a 18% polysaccharide solution, a 18.5% polysaccharide solution, a 19% polysaccharide solution, a 19.5% polysaccharide solution, a 20% polysaccharide solution, a 21% polysaccharide solution, a 22% polysaccharide solution, a 23% polysaccharide solution, a 24% polysaccharide solution, a 25% polysaccharide solution, a 26% polysaccharide solution, a 28% polysaccharide solution, a 29% polysaccharide solution, a 30% polysaccharide solution, or any combination thereof. In some embodiments, the plurality of polysaccharide solutions comprises: a 12.5% polysaccharide solution, a 15% polysaccharide solution, a 16% polysaccharide solution, a 17% polysaccharide solution, a 25% polysaccharide solution, or any combination thereof. In some embodiments, the plurality of polysaccharide solutions comprises 12.5% polysaccharide solution, 15% polysaccharide solution, and 17% polysaccharide solution as shown in FIG. 3A. In some embodiments, the population of cells accumulates at a density range of about 14% polysaccharide solution, about 14.5% polysaccharide solution, about 15% polysaccharide solution, about 15.5% polysaccharide solution, or about 16% polysaccharide solution. In some embodiments, the population of cells accumulates at a density range of about 15% polysaccharide. In some embodiments, the polysaccharide density gradient comprises a Ficoll density gradient. In some embodiments, the plurality of cells comprises a heterologous polynucleotide. In some embodiments, the method comprises cryopreserving the population of cells. In some embodiments, the method comprises thawing the population of cells, wherein, following the thawing, an enucleated cell of the population of cells is as viable as an otherwise comparable enucleated cell that is not cryopreserved. In some embodiments, the fixed angle centrifugation comprises ultracentrifugation. In some embodiments, the population of cells comprises stem cells. In some embodiments, the stem cells comprise induced pluripotent stem cells (iPSCs), adult stem cells, mesenchymal stromal cells, embryonic stem cells, fibroblasts, immortalized cells from a cell line, or any combination thereof. In some embodiments, the stem cells comprise the mesenchymal stromal cells. In some embodiments, the population of cells comprises immune cells. In some embodiments, the immune cells comprise lymphocytes or natural killer cells. In some embodiments, the plurality of cells comprises a heterologous polynucleotide and wherein the exogenous polypeptide is encoded by the heterologous polynucleotide. In some embodiments, the exogenous polypeptide comprises a therapeutic agent. In some embodiments, the population of cells comprise at least one targeting moiety, at least one fusogenic moiety, at least one immune evasion moiety, or a combination thereof.

In some embodiments, the methods of enucleation disclosed herein result in a composition comprising the enucleated cells (also referred to herein as “enucleated cell fraction” of the composition). In some embodiments, the composition further comprises less than or equal to about one (1) percent (%) residual nucleated cells (also referred to herein as “nucleated cell fraction” of the composition) by volume that were not enucleated. In some embodiments, the nucleated cell fraction comprises less than or equal to about 0.1%, 0.2%, 0.3%, 0.4%, 0.5%, 0.6%, 0.7%, 0.8%, or 0.9% of the composition by volume. In some embodiments, the nucleated cell fraction comprises 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, or about 0.1% to about 1.0% of the composition by volume. In some embodiments, the nucleated cell fraction comprises about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, or about 0.2% to about 1.0% of the composition by volume. In some embodiments, the nucleated cell fraction comprises about 0.3% to about 0.4%, about 0.3% to about 0.5%, about 0.3% to about 0.6%, about 0.3% to about 0.7%, about 0.3% to about 0.8%, about 0.3% to about 0.9%, or about 0.3% to about 1.0% of the composition by volume. In some embodiments, the nucleated cell fraction comprises about 0.4% to about 0.5%, about 0.4% to about 0.6%, about 0.4% to about 0.7%, about 0.4% to about 0.8%, about 0.4% to about 0.9%, or about 0.4% to about 1.0% of the composition by volume. In some embodiments, the nucleated cell fraction comprises about 0.5% to about 0.6%, about 0.5% to about 0.7%, about 0.5% to about 0.8%, about 0.5% to about 0.9%, or about 0.5% to about 1.0% of the composition by volume. In some embodiments, the nucleated cell fraction comprises about 0.6% to about 0.7%, about 0.6% to about 0.8%, about 0.6% to about 0.9%, or about 0.6% to about 1.0% of the composition by volume. In some embodiments, the nucleated cell fraction comprises about 0.7% to about 0.8%, about 0.7% to about 0.9%, or about 0.7% to about 1.0% of the composition by volume. In some embodiments, the nucleated cell fraction comprises about 0.8% to about 0.9%, or about 0.8% to about 1.0% of the composition by volume. In some embodiments, the nucleated cell fraction comprises about 0.9% to about 1.0% of the composition by volume.

Also described herein are pharmaceutical compositions and formulations comprising the compositions described herein, and a pharmaceutically acceptable: carrier, excipient, diluent, or nebulized inhalant. The pharmaceutical compositions are provided in pharmaceutical formulations. In some embodiments, the pharmaceutical formulations are formulated for administration to a subject as a combination therapy (e.g., prodrug, adjuvant, additional therapeutic agent, or other therapy) or monotherapy. In some embodiments, the pharmaceutical formulations are formulated for systemic administration or at the site of action, such as intratumoral administration.

Disclosed herein are kits comprising the composition disclosed herein and packaging material configured to deliver the composition to an individual. The kits disclosed herein may comprise a composition comprising an enucleated cell fraction and less than 0.1% nucleated cell fraction. In some embodiments, the kits further comprise instructions for further engineering the enucleated cells in the enucleated cell fraction, such as for example, to produce or secrete a therapeutic agent disclosed herein. In some cases, the instructions may further comprise instructions for how to formulate the resulting composition into a pharmaceutical formulation for administration to a subject disclosed herein.

### Compositions

Disclosed herein are compositions and formulations thereof comprising enucleated cells capable of being extensively engineered to express an active agent, or portion thereof, in the absence of a nucleus. Such enucleated cells are viable cell-like entities capable of synthesizing, releasing (e.g., secreting), or delivering the active agent to a target cell or tissue in the absence of the nucleus. The compositions disclosed herein can be stored in a suspended biological stage by means such as cryohibernation, cryopreservation, or lyophilization for any period of time without impacting the viability of the enucleated cell once the biological activity is revived. Moreover, the compositions disclosed herein comprise less than or equal to about 0.1% of nucleated cells (e.g., parent cells that were not enucleated during the enucleation process), rendering the compositions disclosed herein optimal for therapeutic applications. The enucleated cells (as referred to here as “cytoplasts”) may further comprise naturally occurring cell-surface molecules retained from the parent cell. In some embodiments, the enucleated cells further comprise exogenous molecules, such as a targeting moiety, a transmembrane moiety, an additional therapeutic agent (e.g., other than the active agent) such as those disclosed herein.

### Enucleated Cell

The enucleated cells of the present disclosure are obtained or derived from a corresponding nucleated cell (referred to herein as a “parent cell”). The parent cell may be derived from a variety of different cell types, including eukaryotic cells. For example, an enucleated cell may be derived from an adult stem cell, a mesenchymal stromal cell (MSC), a natural killer (NK) cell, a macrophage, a myoblast, a neutrophil, endothelial cell, endothelial precursor cell, and/or a fibroblast. In some embodiments, an enucleated cell is derived from a mesenchymal stromal cell. In some embodiments, the enucleated cell is derived from an inducible pluripotent stem cell (iPSC). In some embodiments, the parent cell is derived from a cell is immortalized using suitable methods. In some embodiments, the enucleated cell comprises or retains one or more structural features of the parent cell, including intracellular organelles, one or more tunneling nanotubes, or a combination thereof. In some embodiments, the enucleated cell comprises one or more intracellular organelles for synthesis or secretion of an exogenous polypeptide (e.g., therapeutic agent) in absence of the nucleus. In some embodiments, the one or more intracellular organelles comprise a Golgi apparatus, an endoplasmic reticulum, or a combination thereof. In some embodiments, the enucleated cell comprises or expresses any one of the therapeutic agents described herein.

In some embodiments, the cell can originate from any organism having one or more cells. Non-limiting examples of cells include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell). In some embodiments, the cell is a somatic cell. In some embodiments, the cell is a stem cell or a progenitor cell. In some embodiments, the cell is a mesenchymal stem or progenitor cell. In some embodiments, the cell is a hematopoietic stem or progenitor cell. In some embodiments, the cell is a muscle cell, a skin cell, a blood cell, or an immune cell. Other non-limiting example of cells includes lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

In some embodiments, the cell is a eukaryotic cell. Non-limiting examples of eukaryotic cells include mammalian (e.g., rodent, non-human primate, or human), non-mammalian animal (e.g., fish, bird, reptile, or amphibian), invertebrate, insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. In some embodiments, the nucleated cell is a primary cell. In some embodiments, the nucleated cell is an immune cell (e.g., a lymphocyte (e.g., a T cell, a B cell), a macrophage, a natural killer cell, a neutrophil, a mast cell, a basophil, a dendritic cell, a monocyte, a myeloid-derived suppressor cell, an eosinophil). In some embodiments, the nucleated cell is a phagocyte or a leukocyte. In some embodiments, the nucleated cell is a stem cell (e.g., an adult stem cell (e.g., a hematopoietic stem cell, a mammary stem cell, an intestinal stem cell, mesenchymal stem cell, an endothelial stem cell, a neural stem cell, an olfactory adult stem cell, a neural crest stem cell, a testicular cell), an embryonic stem cell, an inducible pluripotent stem cell (iPS)). In some embodiments, the nucleated cell is a progenitor cell. In some embodiments, the nucleated cell is from a cell line. In some embodiments, the nucleated cell is a suspension cell. In some embodiments, the nucleated cell is an adherent cell. In some embodiments, the nucleated cell is a cell that has been immortalized by expression of an oncogene. In some embodiments, the nucleated cell is immortalized by the expression of human telomerase reverse transcriptase (hTERT) or any oncogene. In some embodiments, the nucleated cell is a patient or subject derived cell (e.g., an autologous patient-derived cell, or an allogenic patient-derived cell). In some embodiments, the nucleated cell is transfected with a vector (e.g., a viral vector (e.g., a retrovirus vector (e.g., a lentivirus vector), an adeno-associated virus (AAV) vector, a vesicular virus vector (e.g., vesicular stomatitis virus (VSV) vector), or a hybrid virus vector), a plasmid) before the nucleated cell is enucleated using any of the enucleation techniques described herein and known in the art.

In some embodiments, the cytoplast is derived from a cell autologous to the subject. In some embodiments, the cytoplast is derived from a cell allogenic to the subject.

In some embodiments, the cytoplast is derived from an immune cell. In some embodiments, the cytoplast is derived from a natural killer (NK) cell, a neutrophil, a macrophage, a lymphocyte, a fibroblast, an adult stem cell (e.g., hematopoietic stem cell, a mammary stem cell, an intestinal stem cell, a mesenchymal stem cell, a mesenchymal stromal cell, an endothelial stem cell, a neural stem cell, an olfactory adult stem cell, a neural crest stem cell, a skin stem cell, or a testicular cell), a mast cell, a basophil, an eosinophil, an endothelial cell, an endothelial cell precursor cell, or an inducible pluripotent stem cell.

In some embodiments, the parent cell may be enucleated and engineered for therapeutic use. In some embodiments, a parent cell may be treated with cytochalasin to soften the cortical actin cytoskeleton. In some embodiments, the nucleus is then physically extracted from the cell body by high-speed centrifugation in gradients of polysaccharide to generate an enucleated cell. In some embodiments, the polysaccharide is Ficoll for generating Ficoll gradients to generate an enucleated cell. Because enucleate cells and intact nucleated cells sediment to different layers in the Ficoll gradient, enucleated cells may be isolated and prepared for therapeutic purposes or fusion to other cells (nucleated or enucleated). The enucleation process can be clinically scalable to process tens of millions of cells by utilizing the methods described herein. In some embodiments, enucleated cells may be used as a disease-homing vehicle to deliver clinically relevant cargos or payloads to treat various diseases or conditions described herein.

In some embodiments, the enucleated cell comprises at least one therapeutic agent. In some embodiments, the enucleated cells disclosed herein express the therapeutic agent with one or more intracellular organelles in the absence of the nucleus. In some embodiments, the therapeutic agent is exogenous to the enucleated cell or parent (nucleated) cell thereof. In some embodiments, the enucleated cell expresses the therapeutic agent at the surface of the enucleated cell. In some embodiments, the therapeutic agent is secreted by the enucleated cell into extracellular space at a target tissue (e.g., a microenvironment). In some embodiments, the therapeutic agent is cargo (e.g., encapsulated by the enucleated cell) of the enucleated cell.

In some embodiments, the enucleated cell is obtained from a first subset of a plurality of nucleated cells. In some embodiments, the enucleated cells are in a composition, which further comprises a second subset of the plurality of the nucleated cells. In some embodiments, the second subset of the nucleated cells comprises less than about 0.1% by volume of the composition. In some embodiments, the second subset of the nucleated cells comprises less than about 0.5% by volume of the composition. In some embodiments, the second subset of the nucleated cells comprises less than about 1% by volume of the composition. In some embodiments, the second subset of the nucleated cells comprises less than about 5% by volume of the composition. In some embodiments, the second subset of the nucleated cells comprises less than about 10% by volume of the composition. In some embodiments, the second subset of the nucleated cells comprises less than about 15% by volume of the composition. In some embodiments, the second subset of the nucleated cells comprises less than about 20% by volume of the composition. In some embodiments, the second subset of the nucleated cells comprises less than about 25% by volume of the composition. In some embodiments, the second subset of the nucleated cells comprises less than about 30% by volume of the composition. In some embodiments, the second subset of the nucleated cells comprises less than about 40% by volume of the composition. In some embodiments, the second subset of the nucleated cells comprises less than about 50% by volume of the composition.

In some embodiments, the enucleated cell described herein can be cryopreserved, cryohibernated, lyophilized, or a combination thereof. In some embodiments, the cryopreserved enucleated cell, following thawing, the enucleated cell is as viable as an otherwise comparable enucleated cell that is not cryopreserved. In some embodiments, the lyophilized enucleated cell is as viable as an otherwise comparable enucleated cell that is not lyophilized. In some embodiments, the cryohibernated enucleated cell is as viable as an otherwise comparable enucleated cell that is not cryohibernated. In some embodiments, the cryohibernation comprises storing the enucleated cells at a temperature that is below room temperature but without freezing the enucleated cell. In some embodiments, the cryohibernation comprises storing the enucleated cells at about 4° C. In some embodiments, the cryohibernation comprises storing the enucleated cells at most about 4° C. In some embodiments, the cryohibernation comprises storing the enucleated cells for at least about 24 hours, for at least about 48 hours, at least about 72 hours, at least 96 about hours, at least about 5 days, at least about 6 days, at least about 7 days, at least about 10 days, at least about 15 days, at least about one month, at least about one month, or at least about one year. In some embodiments, the cryohibernation comprises contacting or storing the enucleated cells in a media such as a cell culture media. In some embodiments, the cell culture media comprises xenofree media. In some embodiments, the cell culture media comprises about 5% to about 20% serum.

In some embodiments, the enucleated cell or the composition comprising the enucleated cell may be cryopreserved (e.g., storing the enucleated cell or the composition comprising the enucleated cell at freezing temperature) or cryohibernated (e.g., storing the enucleated cell or the composition comprising the enucleated cell at a temperature that is between the ambient temperature and freezing temperature). The duration of cryopreservation or cryohibernation may be greater than or equal to about one hour, two hours, six hours, 12 hours, one day, two days, three days, four days, five days, six days, one week, two weeks, three weeks, four weeks, one month, two months, three months, or longer period of time. In some embodiments, the enucleated cell exhibits a viability after cryopreservation or cryohibernation that is greater than or equal to about 50%, 55%, 60%, 65%, 70%, 75%, 80%, 85%, 90%, 95%, or 99% similar to a comparable cell (e.g., a parent cell or an enucleated cell described herein that has not been cryopreserved or cryohibernated) after the same period of time of cryopreservation or cryohibernation. In some embodiments, the enucleated cell exhibits the viability following the cryohibernation as measured at 24 hours following the cryohibernation that is equal to or greater than the viability of a comparable enucleated cell that is not cryohibernated. In some embodiments, the enucleated cell exhibits the viability following the cryopreservation as measured at 24 hours following the cryopreservation that is equal to or greater than the viability of a comparable enucleated cell that is not cryopreserved. Viability in this context may be measured by Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, the viability is measured using Annexin-V cell surface staining. In some embodiments, the viability is measured by expression of the exogenous polypeptide. For example, the viability of the enucleated cell can be determined by the expression of the exogenous antibody or single-domain antibody expressed by the enucleated cell. In some embodiments, the viability is measured by expression of cell surface markers of any one of the cell surface markers described herein such as CD105, CD90, CD45, CXCR4, PSGL-1, or CCR2. In some embodiments, the viability is measured by the cell activity of the enucleated cell. In some embodiments, the viability is measured by the homing capability of the enucleated cell as determined by the chemosensing or chemokine homing activity described herein.

In some embodiments, the enucleated cell or the composition comprising the enucleated cell may be lyophilized. In some embodiments, the enucleated cell exhibits a viability after being reconstituted from lyophilization that is greater than or equal to about 50%, 55%, 60%, 65%, 70%, 75%, 80%, 85%, 90%, 95%, or 99% similar to a comparable cell (e.g., a parent cell or an enucleated cell described herein that has not been lyophilized). In some embodiments, the enucleated cell exhibits a viability after being rehydrated from lyophilization that is greater than or equal to about 50%, 55%, 60%, 65%, 70%, 75%, 80%, 85%, 90%, 95%, or 99% similar to a comparable cell (e.g., a parent cell or an enucleated cell described herein that has not been dehydrated).

In some embodiments, the enucleated cell or the composition comprising the enucleated cell may be dehydrated. In some embodiments, the enucleated cell exhibits a viability after being rehydrated that is greater than or equal to about 50%, 55%, 60%, 65%, 70%, 75%, 80%, 85%, 90%, 95%, or 99% similar to a comparable cell (e.g., a parent cell or an enucleated cell described herein that has not been dehydrated).

In some embodiments, the enucleated cell or the composition comprising the enucleated cell is stable at 4° C. for greater than or equal to about one hour, two hours, six hours, 12 hours, one day, two days, three days, four days, five days, six days, one week, two weeks, three weeks, four weeks, one month, two months, three months, or longer period of time. In some embodiments, the composition is stable at room temperature for greater than or equal to about one hour, two hours, six hours, 12 hours, one day, two days, three days, four days, five days, six days, one week, two weeks, three weeks, four weeks, one month, two months, three months, or longer period of time. In some embodiments, the composition is stable at 37° C. for greater than or equal to about one hour, two hours, six hours, 12 hours, one day, two days, three days, four days, five days, six days, one week, two weeks, three weeks, four weeks, one month, two months, three months, or longer period of time. In some embodiments, the enucleated cell or the composition comprising the enucleated cell may remain viable after being administered to a subject in need thereof for treating the disease or condition described herein. In some embodiments, the enucleated cell or the composition comprising the enucleated cell may remain viable after being administered to the subject for greater than or equal to about one hour, two hours, six hours, 12 hours, one day, two days, three days, four days, five days, six days, one week, two weeks, three weeks, four weeks, one month, two months, three months, or longer period of time.

In some embodiments, the enucleated cell may be obtained from a parent cell that is autologous to the subject, who is in need of the treatment by the enucleated cell described herein. In some embodiments, the enucleated cell may be obtained from a parent cell that is allogenic to the subject, who is in need of the treatment by the enucleated cell described herein.

Enucleated cells may be smaller than their nucleated counterparts (e.g., the nucleated parent cells), and for this reason the enucleated cells may migrate better through small openings in the vasculature and tissue parenchyma. In addition, removing the large dense nucleus alleviates a major physical barrier allowing the cell to move freely through small openings in the vessels and tissue parenchyma. Therefore, enucleated cells have improved bio-distribution in the body and movement into target tissues. In some embodiments, an enucleated cell comprises at least 1 μm in diameter. In some embodiments, an enucleated cell is greater than 1 μm in diameter. In some embodiments, an enucleated cell is 1-100 μm in diameter (e.g., 1-90 μm, 1-80 m, 1-70 μm, 1-60 μm, 1-50 μm, 1-40 μm, 1-30 am, 1-20 am, 1-10 μm, 1-5 μm, 5-90 μm, 5-80 m, 5-70 μm, 5-60 μm, 5-50 μm, 5-40 μm, 5-30 μm, 5-20 μm, 5-10 μm, 10-90 μm, 10-80 μm, 10-70 μm, 10-60 μm, 10-50 μm, 10-40 μm, 10-30 μm, 10-20 μm, 10-15 μm 15-90 μm, 15-80 μm, 15-70 μm, 15-60 μm, 15-50 μm, 15-40 μm, 15-30 μm, 15-20 m). In some embodiments, an enucleated cell is 10-30 μm in diameter. In some embodiments, the diameter of an enucleated cell is between 5-25 μm (e.g., 5-20 μm, 5-15 μm, 5-10 μm, 10-25 μm, 10-20 μm, 10-15 μm, 15-25 μm, 15-20 μm, or 20-25 m). In some embodiments, the enucleated cell has a diameter that is more than or equal to about 5 μm, about 10 μm, about 20 μm, about 30 μm, about 40 μm, about 50 μm, about 60 μm, about 70 μm, about 80 μm, about 90 μm, or about 100 μm. In some embodiments, the enucleated cell has a diameter that is more than or equal to about 5 μm, about 6 μm, about 7 μm, about 8 μm, about 9 μm, about 10 μm, about 11 μm, or about 12 μm. In some embodiments, the enucleated cell has a diameter that is about 8 μm. In some embodiments, some enucleated cells may advantageously be small enough to allow for better homing or delivery to a target site. For examples, the enucleated cells described herein may pass through passages in narrow lung tissues or lung structures such as alveolar duct or microcapillary that most cells such as the parent cells may not pass through.

In some embodiments, the enucleated cell of the enucleated cell fraction has a diameter comprising less than or equal to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of an average diameter of a nucleated parent cell. In some embodiments, the enucleated cell of the enucleated cell fraction has a diameter comprising less than or equal to about 10%, about 20%, about 30%, about 40%, about 50%, about 60%, about 70%, about 80%, about 90%, about 95%, or about 99% of an average diameter of the nucleated cells. In some embodiments, the enucleated cell of the enucleated cell fraction has a diameter comprising less than or equal to about 50% of an average diameter of the nucleated cells. In some embodiments, the enucleated cell of the enucleated cell fraction has a diameter comprising less than or equal to about 60% of an average diameter of the nucleated cells. In some embodiments, the enucleated cell of the enucleated cell fraction has a diameter comprising less than or equal to about 70% of an average diameter of the nucleated cells. In some embodiments, the enucleated cell of the enucleated cell fraction has a diameter comprising less than or equal to about 80% of an average diameter of the nucleated cells. In some embodiments, the enucleated cell of the enucleated cell fraction has a diameter comprising less than or equal to about 90% of an average diameter of the nucleated cells.

In some embodiments, enucleated cells possess significant therapeutic value, because they remain viable, do not differentiate into other cell types, secrete bioactive molecules, and may physically migrate/home for fewer than or equal to about 5 days, may be extensively enucleated ex vivo to perform specific therapeutic functions, and may be fused to the same or other cell types to transfer desirable production, natural or enucleated. Therefore, enucleated cells have wide utility as a cellular vehicle to deliver therapeutically important biomolecules and disease-targeting cargos including genes, viruses, bacteria, mRNAs, shRNAs, siRNA, polypeptides (including antibodies and antigen binding fragments), plasmids, gene-editing machinery, or nanoparticles. The present disclosure enables the generation of safe (e.g., no unwanted DNA is transferred to the subject), and controllable cell-based carrier that may be genetically enucleated to deliver specific disease-fighting and health promoting cargos to humans. In some embodiments, the enucleated cell remains viable and retain the function to migrate or home for greater than or equal to about 12 hours, 24 hours, 36 hours, 48 hours, 60 hours, 72 hours, 84 hours, 96 hours, 108 hours, 5 days, 6 days, 7 days, 8 days, 9 days, or longer after being administered to the subject in need thereof.

In some embodiments, the enucleated cell is engineered to express at least one of an exogenous DNA molecule, an exogenous RNA molecule, an exogenous protein, or an exogenous protein, gene-editing machinery or combinations thereof. In some embodiments, the exogenous DNA molecule is a single-stranded DNA, a double-stranded DNA, an oligonucleotide, a plasmid, a bacterial DNA molecule, a DNA virus, linear DNA, or combinations thereof. In some embodiments, the exogenous RNA molecule is messenger RNA (mRNA), small interfering RNA (siRNA), microRNA (miRNA), short hairpin RNA (shRNA), an RNA virus, or combinations thereof. In some embodiments, the exogenous protein is a cytokine, a growth factor, a hormone, an antibody, or the antigen-binding fragment thereof, an enzyme, or combinations thereof. In some embodiments, the antibody is a single-domain antibody or antigen-binding fragment thereof. In some embodiments, parental cells (e.g., nucleated cells) are genetically enucleated before enucleation (e.g., pre-enucleation). In some embodiments, the parent cell is genetically enucleated after enucleation (e.g., post-enucleation).

### Transmembrane Moiety

Described herein, in some aspects, are enucleated cells or compositions comprising the enucleated cell comprising at least one transmembrane moiety. In some embodiments, the enucleated cell comprises an exogenous polypeptide. The exogenous polypeptide may be covalently fused to a transmembrane moiety. In some embodiments, the exogenous polypeptide is complexed to the transmembrane moiety. In some embodiments, the transmembrane moiety comprises a full length protein or a variation thereof or a fragment thereof. In some embodiments, the transmembrane moiety is endogenous to the parent cell that is being enucleated for obtaining the enucleated cell. In some embodiments, the transmembrane moiety may be an exogenous transmembrane moiety to the parent cell or to the enucleated cell. In some embodiments, the transmembrane moiety is selected from a transmembrane protein comprising a single transmembrane α-helix (bitopic membrane protein). The transmembrane moiety comprises a polytopic transmembrane α-helical protein. In some embodiments, the transmembrane moiety comprises a polytopic transmembrane β-sheet protein. In some embodiments, the transmembrane moiety comprises a Type I, II, III, or IV transmembrane protein. Non-limiting examples of transmembrane protein may include CD4, CD14, glycophorin a (GPA), or any combination of integrins.

In some embodiments, the transmembrane moiety is added to the exogenous polypeptide by way of a modification. For example, a transmembrane moiety may be added to the N or C-terminus of the exogenous polypeptide to insert the exogenous polypeptide into the cell membrane of the enucleated cell described herein. Non-limiting examples of modifications that are made to the exogenous polypeptide to add the transmembrane moiety may include adding glycosylphosphatidylinositol, farnesyl, palmitate, myristate, or a combination thereof to the exogenous polypeptide.

In some embodiments, the transmembrane moiety is genetically modified to be fused or complexed with the at least one exogenous therapeutic agent described herein. In some embodiments, the transmembrane moiety is genetically modified to fuse to the at least one exogenous therapeutic agent described herein. In some embodiments, the enucleated cell comprises an immune-evading moiety. In some embodiments, the immune-evading comprises a “don't eat me” signaling peptide, such as CD47 (e.g., NCBI Gene ID 961), programmed cell death 1 ligand 1 (PD-L1, e.g., NCBI Gene ID 29126), major histocompatibility complex, class I, E (HLA-E, e.g., NCBI Gene ID 3133), major histocompatibility complex, class I, G (HLA-I, e.g., NCBI Gene ID 3135), a fragment thereof, or a combination thereof.

### Targeting Moiety

Described herein, in some aspects, are enucleated cells comprising a targeting moiety. The targeting moiety described herein is designed to guide the enucleated cell to a target cell or target environment (e.g., tissue) in a subject following delivery (e.g., systemic delivery) of the enucleated cell to the subject. In some embodiments, the targeting moiety is expressed on the surface of the enucleated cell. In some embodiments, the targeting moiety is complexed with a transmembrane moiety described herein. In some embodiments, the targeting moiety is secreted by the enucleated cell. In some embodiments, the enucleated cell comprising the targeting moiety localizes at the target cell or target environment with a 2-fold, 5-fold, 10-fold, 50-fold, 100-fold, 200-fold, 500-fold, 1,000-fold, 5,000-fold, or 10,000-fold increase as compared to localization of a comparable enucleated cell lacking the targeting moiety. In some embodiments, the enucleated cell comprising the targeting moiety localizes at the target cell or target environment with an increase of 50%, 55%, 60%, 65%, 70%, 75%, 80%, 85%, 90%, 95%, or 99% as compared with a comparable enucleated cell lacking the targeting moiety. In some embodiments, the target cell or target environment is in vivo. In some embodiments, the target cell or target environment is ex vivo.

In some embodiments, the targeting moiety comprises an exogenous antibody or an exogenous antigen-binding fragment for targeting a biomarker described herein. In some embodiments, the targeting moiety comprises an exogenous antibody or an exogenous antigen-binding fragment for targeting a chemokine receptor or a chemokine ligand, or portion thereof, involved in chemokine signaling. In some embodiments, the exogenous antibody is an exogenous single-domain antibody or fragment thereof.

In some embodiments, the targeting moiety targets the biomarker expressed by, or associated with, a target cell or with a microenvironment. In some embodiments, the biomarker may be released by the target cell. The biomarker may indicate the presence of the disease or the condition. In some embodiments, the biomarker is expressed by immune cells responding to the target cell or the microenvironment associated with the disease or the condition. In some embodiments, the biomarker may be an epitope or antigen. In some embodiments, the biomarker comprising the epitope may be bound by an antibody that is different from the antibody or the antigen-binding fragment thereof that confers therapeutic property (e.g., the therapeutic agent).

In some embodiments, the targeting moiety targets a biomarker expressed or released by a lung cell or a lung cancer cell. Non-limiting example of cancer cell biomarkers includes carbonic anhydrase 9 (CA9, e.g., NCBI Gene ID 768), carbonic anhydrase 12 (CA12, e.g., NCBI Gene ID 771), cancer/testis antigen 83 (CXorf61; e.g., NCBI Gene ID203413), desmoglein 3 (DSG3 (e.g., NCBI Gene ID 1830), FAT atypical cadherin 2 (FAT2 (e.g., NCBI Gene ID 2196), G protein-coupled receptor 87 (GPR87, e.g., NCBI Gene ID 53836), KISS1 receptor (KISSIR, e.g., NCBI Gene ID 84634), LY6/PLAUR domain containing 3 (LYPD3. e.g., NCBI Gene ID 27076), solute carrier family 7 member 11 (SLC7A11, e.g., NCBI Gene ID 23657), TMPRSS4 (e.g., NCBI Gene ID 56649), transmembrane serine protease 4 (TFPI, e.g., NCBI Gene ID 7035), midkine (MDK, e.g., NCBI Gene ID 4192), secreted phosphoprotein 1 (OPN, e.g., NCBI Gene ID 6696), matrix metallopeptidase 2 (MMP2, e.g., NCBI Gene ID 4313), TIMP metallopeptidase inhibitor 1 (TIMP1, e.g., NCBI Gene ID 7076), cell adhesion molecule 5 (CEA, e.g., NCBI Gene ID 1048), cytokeratin 19 fragment (CYFRA 21-1, e.g., NCBI Gene ID 3880), serpin family B member 3 (SCC, e.g., NCBI Gene ID 6317), advanced glycosylation end-product specific receptor (AGER, e.g., NCBI Gene ID 177), adipogenesis regulatory factor (C10orf116, e.g., NCBI Gene ID 10974), adducin 2 (ADD2, e.g., NCBI Gene ID 119), periaxin (PRX, e.g., NCBI Gene ID 57716), laminin subunit beta 3 (LAMB3, e.g., NCBI Gene ID 3914), synemin (SYNM, e.g., NCBI Gene ID 23336), spectrin alpha, erythrocytic 1 (SPTA1, e.g., NCBI Gene ID 6708), ankyrin 1 (ANK1, e.g., NCBI Gene ID 286), hemoglobin subunit epsilon 1 (HBEl, e.g., NCBI Gene ID 3046), hemoglobin subunit gamma 1 (HBG1, e.g., NCBI Gene ID 3047), carbonic anhydrase 1 (CAl, e.g., NCBI Gene ID 759), tenascin XB (TNXB, e.g., NCBI Gene ID 7148), multimerin 2 (MMRN2, e.g., NCBI Gene ID 79812), hemoglobin subunit alpha 1 (HBA1, e.g., NCBI Gene ID 3039), caveolin 1 (CAV1, e.g., NCBI Gene ID 857), hemoglobin subunit beta (HBB, e.g., NCBI Gene ID 3043), collagen type VI alpha 6 chain (COL6A6, e.g., NCBI Gene ID 131873), chromosome 1 open reading frame 198 (Clorfl98, e.g., NCBI Gene ID 84886), chloride intracellular channel 2 (CLIC2, e.g., NCBI Gene ID 1193), transcriptional regulator of SdpC synthesis operon (ArsR family) (SDPR, e.g., NCBI Gene ID 8436), EH domain containing 2 (EHD2, e.g., NCBI Gene ID 30846), apolipoprotein A2 (APOA2, e.g., NCBI Gene ID 336), NADH: ubiquinone oxidoreductase subunit B7 (NDUFB7, e.g., NCBI Gene ID 4713), protein kinase C delta binding protein (PRKCDBP, e.g., NCBI Gene ID 112464), laminin subunit alpha 3 (LAMA3, e.g., NCBI Gene ID), EvC ciliary complex subunit 2 (LBN, e.g., NCBI Gene ID 132884), serpin family A member 3 (ACT, e.g., NCBI Gene ID 12), insulin like growth factor binding protein 3 (3 IGFBP3, e.g., NCBI Gene ID 3486), prostaglandin D2 synthase (L-PGDS, e.g., NCBI Gene ID 5730), retinoic acid receptor beta (HAP, e.g., NCBI Gene ID 5915), hepatocyte growth factor (HGF, e.g., NCBI Gene ID 3082), eukaryotic translation initiation factor 4 gamma 2 (AAG1/2, e.g., NCBI Gene ID 1982), clusterin (CLU, e.g., NCBI Gene ID 1191), streptococcal superantigen SSA (SSA, e.g., NCBI Gene ID 6737), tetanic (TTA, e.g., NCBI Gene ID 100189453), apolipoprotein A4 (APOA4, e.g., NCBI Gene ID 337), fibrinogen-like protein A (FIBA, e.g., NCBI Gene ID 105209070), serum amyloid A cluster (SAA, e.g., NCBI Gene ID 6288), ceruloplasmin (CP, e.g., NCBI Gene ID 1356), haptoglobin (HP, e.g., NCBI Gene ID 3240), transthyretin (TTR, e.g., NCBI Gene ID 7276), keratin 2 (KRT2A, e.g., NCBI Gene ID 3849), glutamate transporter (GLT1B, e.g., NCBI Gene ID 6506), casein kinase 1 (CK1, e.g., NCBI Gene ID 1452), AKT serine/threonine kinase 1 (AKT, e.g., NCBI Gene ID 207), mannose binding lectin 2 (MBL2, e.g., NCBI Gene ID 4153), fibrinogen alpha chain (FGA, e.g., NCBI Gene ID 2243), gelsolin (GSN, e.g., NCBI Gene ID 2934), haptoglobin (HP, e.g., NCBI Gene ID 3240), ficolin 3 (FCN3, e.g., NCBI Gene ID 8547), carnosine dipeptidase 1 (CNDP1, e.g., NCBI Gene ID 84735), calcitonin related polypeptide alpha (CALCA, e.g., NCBI Gene ID 796), carbamoyl-phosphate synthase 1 (CPS1, e.g., NCBI Gene ID 1373), chromogranin B (CHGB, e.g., NCBI Gene ID 1114), involucrin (IVL, e.g., NCBI Gene ID 3713), anterior gradient 2 (AGR2, e.g., NCBI Gene ID 10551), nuclear autoantigenic sperm protein (NASP, e.g., NCBI Gene ID 4678), phosphofructokinase, platelet (PFKP, e.g., NCBI Gene ID 5214), thrombospondin 2 (THBS2, e.g., NCBI Gene ID 7058), thioredoxin domain containing 17 (TXNDC17, e.g., NCBI Gene ID 84817), proprotein convertase subtilisin/kexin type 1 (PCSK1, e.g., NCBI Gene ID 5122), cellular retinoic acid binding protein 2 (CRABP2, e.g., NCBI Gene ID 1382), acyl-CoA binding domain containing 3 (ACBD3, e.g., NCBI Gene ID 64746), desmoglein 2 (DSG2, e.g., NCBI Gene ID 1829), LPS responsive beige-like anchor protein (LRBA, e.g., NCBI Gene ID 987), serine/threonine kinase receptor associated protein (STRAP, e.g., NCBI Gene ID 11171), VGF nerve growth factor inducible (VGF, e.g., NCBI Gene ID 7425), NOP2 nucleolar protein (NOP2, e.g., NCBI Gene ID 4839), lipocalin 2 (LCN2, e.g., NCBI Gene ID 3934), creatine kinase, mitochondrial 1B (CKMTlB, e.g., NCBI Gene ID 1159), aldo-keto reductase family 1 member B10 (AKR1B10, e.g., NCBI Gene ID 57016), carboxypeptidase D (CPD, e.g., NCBI Gene ID 1362), proteasome activator subunit 3 (PSME3, e.g., NCBI Gene ID 10197), villin 1 (VIL1, e.g., NCBI Gene ID 7429), serpin family B member 5 (SERPINB5, e.g., NCBI Gene ID 5268), ribosomal protein L5 (RPL5, e.g., NCBI Gene ID 6125), plakophilin 1 (PKP1, e.g., NCBI Gene ID 5317), ribosomal protein L10 (RPL10, e.g., NCBI Gene ID 6134), aldo-keto reductase family 1 member B10 (AKR1B10, e.g., NCBI Gene ID 57016), aldo-keto reductase family 1 member C1 (AKR1C1, e.g., NCBI Gene ID 1645), proliferating cell nuclear antigen (PCNA, e.g., NCBI Gene ID 5111), ribosomal protein S2 (RPS2, e.g., NCBI Gene ID 6187), aldo-keto reductase family 1 member C3 (AKR1C3, e.g., NCBI Gene ID 8644), acyl-CoA binding domain containing 3 (ACBD3, e.g., NCBI Gene ID 64746), visinin like 1 (VSNL1, e.g., NCBI Gene ID 7447), adenosylhomocysteinase (AHCY, e.g., NCBI Gene ID 191), IMMP10, activated kinase 2 (PAK2, e.g., NCBI Gene ID 5062), involucrin (IVL, e.g., NCBI Gene ID 3713), isoleucine-tRNA synthetase (IARS, e.g., NCBI Gene ID 3376), proteasome 26S subunit ubiquitin receptor, non-ATPase 2 (PSMD2, e.g., NCBI Gene ID 5708), guanylate binding protein 5 (GBP5, e.g., NCBI Gene ID 115362), minichromosome maintenance complex component 6 (MCM6, e.g., NCBI Gene ID 4175), N-myc downstream regulated 1 (NDRG1, e.g., NCBI Gene ID 10397), NOP58 ribonucleoprotein (NOP58, e.g., NCBI Gene ID 51602), S100 calcium binding protein A2 (S100A2, e.g., NCBI Gene ID 6273), neuregulin 1 (NRG1, e.g., NCBI Gene ID 3084), neuregulin 2 (NRG2, e.g., NCBI Gene ID 9542), carnosine dipeptidase 1 (CNDP1, e.g., NCBI Gene ID 84735), ubiquitin cross-reactive protein (UCRP, e.g., NCBI Gene ID 9636), crammer (CER, e.g., NCBI Gene ID 8110), plasminogen activator (UPA, e.g., NCBI Gene ID 5328), matrix metallopeptidase 14 (MT1-MMP, e.g., NCBI Gene ID 4323), stratifin (SFN, e.g., NCBI Gene ID 2810), transferrin (TF, e.g., NCBI Gene ID 7018), albumin (ALB, e.g., NCBI Gene ID 213), S100 calcium binding protein A9 (S100A9, e.g., NCBI Gene ID 6280), stathmin 1 (STMN, e.g., NCBI Gene ID 3925), Enolase (ENO), plasminogen activator (PLAU, e.g., NCBI Gene ID 5328), insulin like growth factor binding protein 7 (IGFBP7, e.g., NCBI Gene ID 3490), matrix metallopeptidase 14 (MMP14, e.g., NCBI Gene ID 4323), thrombospondin 1 (THBS1, e.g., NCBI Gene ID 7057), or thrombospondin 2 (THBS2, e.g., NCBI Gene ID 7058).

In some embodiments, the targeting moiety targets a biomarker expressed or released by a cancer cell that has metastasized. For example, the cancer cell may arise from one tissue and subsequently metastasizes to a different location. In some embodiments, the metastasized cancer cell expresses the non-limiting example of cancer biomarker described herein. In some embodiments, the metastasized cancer cell expresses cancer biomarker includes Melanoma Associated Antigen (MAGE family member A3 (MAGE-A3, e.g., NCBI Gene ID 4102)), Membrane associated glycoprotein (MUC-1, e.g., NCBI Gene ID 4582), glycoproteine-epithelial cell adhesion molecule (EpCAM, e.g., NCBI Gene ID 4072), KRAS Proto-Oncogene (KRAS, e.g., NCBI Gene ID 3845), Anaplastic lymphoma kinase (ALK, e.g., NCBI Gene ID 238), Cytotoxic T-Lymphocyte Associated Protein 4 (CTLA-4, e.g., NCBI Gene ID 1493), Programmed cell death protein 1 (PD-1, e.g., NCBI Gene ID 5133), Epidermal growth factor (EGF, e.g., NCBI Gene ID 1950), Serine protease ester (EA, e.g., NCBI Gene ID 5328), Telomerase reverse transcriptase (TERT, e.g., NCBI Gene ID 7015), PRAME Nuclear Receptor Transcriptional Regulator (PRAME, e.g., NCBI Gene ID 23532), Receptor tyrosine-protein kinase erbB-2 (HER, e.g., NCBI Gene ID 2064), or Vascular endothelial growth factor (VEGF, e.g., NCBI Gene ID 7422), Carcinoembryonic antigen (CEA, e.g., NCBI Gene ID 1048), MAGE family member A1 (MAGE-A1, e.g., NCBI Gene ID 4100), MAGE family member A1 MAGE-A4, e.g., NCBI Gene ID 4103), Survivin, Six Transmembrane Epithelial Antigene of the Prostate 1 (STEAPI, e.g., NCBI Gene ID 26872), SRY (sex determining region Y)-box 2 (SOX2, e.g., NCBI Gene ID 6657), or Cancer/testis antigen 1 (CTAG1B, e.g., NCBI Gene ID 1485).

In some embodiments, the targeting moiety targets a biomarker expressed or released by an endothelial cell. In some embodiments, the endothelial cell is a blood vessel cell. In some embodiments, the endothelial cell is a lymphatic vessel cell. In some embodiments, the biomarker is expressed or released by a blood vessel cell. In some embodiments, the biomarker is expressed or released by a lymphatic vessel cell. Non-limiting examples of the endothelial cell biomarker include angiotensin I converting enzyme (ACE/CD143, e.g., NCBI Gene ID 1636), CD93 molecule (ClqR1/CD93, e.g., NCBI Gene ID 22918), cadherin 5 (VE-Cadherin, e.g., NCBI Gene ID 1003), D6 protein (CC Chemokine Receptor D6, e.g., NCBI Gene ID 1238), platelet and endothelial cell adhesion molecule 1 (CD31/PECAM-1, e.g., NCBI Gene ID 5175), CD34 molecule (CD34, e.g., NCBI Gene ID 947), CD36 molecule (CD36/SR-B3, e.g., NCBI Gene ID 948), CD151 molecule (CD151, e.g., NCBI Gene ID 977), CD160 molecule (CD160, e.g., NCBI Gene ID 11126),

CD300 molecule like family member g (CD300 g/Nepmucin, e.g., NCBI Gene ID 146894), CDC like kinase 1 (CL-K1/COLEC11, e.g., NCBI Gene ID 78989), cleavage factor polyribonucleotide kinase subunit 1 (CL-P1/COLEC12, e.g., NCBI Gene ID 81035), Coagulation Factor III/Tissue Factor (e.g., NCBI Gene ID 2152), C-type lectin domain family 4 member M (DC-SIGNR/CD299, e.g., NCBI Gene ID 10332), discoidin, CUB and LCCL domain containing 2 (DCBLD2/ESDN, e.g., NCBI Gene ID 131566), endothelial cell surface expressed chemotaxis and apoptosis regulator (ECSCR, e.g., NCBI Gene ID 641700), basigin (Ok blood group) (EMMPRIN/CD147, e.g., NCBI Gene ID 682), Endoglin/CD105 (e.g., NCBI Gene ID 5077), Endomucin (e.g., NCBI Gene ID 2022), Endosialin/CD248 (e.g., NCBI Gene ID 57124), protein C receptor (EPCR, e.g., NCBI Gene ID 10544), Erythropoietin R (e.g., NCBI Gene ID 2056), endothelial cell adhesion molecule (ESAM, e.g., NCBI Gene ID 90952), fatty acid binding protein 5 (FABP5/E-FABP, e.g., NCBI Gene ID 2171), fatty acid binding protein 6 (FABP6, e.g., NCBI Gene ID 2172), intercellular adhesion molecule 1 (ICAM-1/CD54, e.g., NCBI Gene ID 3383), intercellular adhesion molecule 2 (ICAM-2/CD102, e.g., NCBI Gene ID 3384), interleukin 1 receptor (IL-1 RI, e.g., NCBI Gene ID 3553), Interleukin 13 receptor, alpha 1 (IL-13 R alpha 1, e.g., NCBI Gene ID 3597), Integrin alpha 4/CD49d (e.g., NCBI Gene ID 3676), Integrin alpha 4 beta 1 (e.g., NCBI Gene ID 3688), Integrin alpha 4 beta 7/LPAM-1 (e.g., NCBI Gene ID 3676), Integrin beta 2/CD18 (e.g., NCBI Gene ID 3689), KLF transcription factor 4 (KLF4, e.g., NCBI Gene ID 9314), lymphatic vessel endothelial hyaluronan receptor 1 (LYVE-1, e.g., NCBI Gene ID 10894), melanoma cell adhesion molecule (MCAM/CD146, e.g., NCBI Gene ID 4162), nectin cell adhesion molecule 2 (Nectin-2/CD112, e.g., NCBI Gene ID 5819), PD-ECGF/Thymidine Phosphorylase (e.g., NCBI Gene ID 1890), Podocalyxin (e.g., NCBI Gene ID 5420), Podoplanin (e.g., NCBI Gene ID 10630), sphingosine-1-phosphate receptor 1 (S1P1/EDG-1, e.g., NCBI Gene ID 1901), sphingosine-1-phosphate receptor 2 (S1P2/EDG-5, e.g., NCBI Gene ID 9294), sphingosine-1-phosphate receptor 3 (S1P3/EDG-3, e.g., NCBI Gene ID 1903), sphingosine-1-phosphate receptor 4 (S1P4/EDG-6, e.g., NCBI Gene ID 8698), sphingosine-1-phosphate receptor 5 (S1P5/EDG-8, e.g., NCBI Gene ID 53637), E-Selectin/CD62E (e.g., NCBI Gene ID 6401), P-Selectin/CD62P (e.g., NCBI Gene ID 6403), slow as molasses (SLAM/CD150, e.g., NCBI Gene ID 6504), Stabilin-1 (e.g., NCBI Gene ID 23166), Stabilin-2 (e.g., NCBI Gene ID 55576), plexin domain containing 1 (TEM7/PLXDC1, e.g., NCBI Gene ID 57125), ANTXR cell adhesion molecule 1 (TEM8/ANTXR1, e.g., NCBI Gene ID 84168), Thrombomodulin/BDCA-3 (e.g., NCBI Gene ID Thrombomodulin), thrombospondin type 1 domain containing 1 (THSD1, e.g., NCBI Gene ID 55901), thrombospondin type 1 domain containing 7A (THSD7A, e.g., NCBI Gene ID 221981), TEK receptor tyrosine kinase (Tie-2, e.g., NCBI Gene ID 7010), TNF receptor superfamily member 1A (TNF RI/TNFRSF1A, e.g., NCBI Gene ID 7132), TNF receptor superfamily member 1B (TNF RII/TNFRSF1B, e.g., NCBI Gene ID 7133), basigin (Ok blood group) (TRA-1-85/CD147, e.g., NCBI Gene ID 682), TNF receptor superfamily member 10b (TRAIL R2/TNFRSF10B, e.g., NCBI Gene ID 8795), TNF receptor superfamily member 10a (TRAILR1/TNFRSF10A, e.g., NCBI Gene ID 8797), vascular cell adhesion molecule 1 (VCAM-1/CD106, e.g., NCBI Gene ID 7412), EGF like domain multiple 7 (VE-Statin, e.g., NCBI Gene ID: 51162), fms related receptor tyrosine kinase 1 (VEGFR1/Flt-1, e.g., NCBI Gene ID 2321), kinase insert domain receptor (VEGFR2/KDR/Flk-1, e.g., NCBI Gene ID 3791), fms related receptor tyrosine kinase 4 (VEGFR3/Flt-4, e.g., NCBI Gene ID 2324), angiogenic factor with G-patch and FHA domains 1 (VG5Q, e.g., NCBI Gene ID 55109), or von Willebrand Factor domain 2 (vWF-A2, e.g., NCBI Gene ID 7450).

In some embodiments, the targeting moiety comprises a chemokine receptor or a chemokine ligand, or portion thereof, involved in chemokine signaling, such as for example, SDF-la/CXCR4, CCL2/CCR2, or adhesion molecules, such as for example, PSGL-1. As shown herein, the enucleated cell may be enucleated to express functional CXCR4, CCR2 as well as glycosylated PSGL-1, which may greatly promote the specific targeting of the enucleated cell. In some embodiments, the targeting moiety, such as CXCR4, CCR2 or PSGL-1 may be expressed on the surface of the enucleated cell. Non-limiting examples of cell surface proteins that may be expressed on the cell surface of the enucleated cell as the targeting moiety include chemokines such as CXCR4, CCR2, CCR1, CCR5, CXCR7, CXCR2, and CXCR1. In some embodiments, the enucleated cell may be enucleated to secrete the targeting moiety or is tethered to the extracellular matrix, e.g., SDF1α or CCL2. Non-limiting examples of targeting moiety that may be secreted by the enucleated cell include SDF1α, CCL2, CCL3, CCL5, CCL8, CCL1, CXCL9, CXCL10, CCL11 and CXCL12. In some embodiments, the enucleated cell comprises cell-matrix receptors and cell-cell adhesion molecules include integrins, cadherins, glycoproteins, and heparin sulfate proteoglycans.

In some embodiments, the enucleated cells may further include (e.g., by engineering or from the cell from which they were obtained) a surface marker that aids in their evasion of the subject immune system. For example, in some embodiments, the enucleated cells may include a CD47, PD-L1, HLA-E, HLA-G, a fragment thereof, or a combination thereof. Without being bound by any particular theory, it is believed that a CD47, PD-L1, HLA-E, HLA-G, a fragment thereof, or a combination thereof helps to prevent the enucleated cells from being phagocytosed by macrophages. Non-limiting examples of cell-matrix receptors and cell-cell adhesion molecules include integrins, cadherins, glycoproteins, or heparin sulfate proteoglycans. In some embodiments, the cell-matrix receptors or cell-cell adhesion molecules include PD-L1, HLA-E, or HLA-G. Non-limiting examples of therapeutic molecules include tumor antigens and immunomodulatory peptides, polyamines, and ATP. In some embodiments, the therapeutic molecules can be recognized by immune cells and can induce immune response. For example, the therapeutic molecules can be 4-1BB or any one of the cytokines described herein to induce immune response.

### Therapeutic Agent

In some embodiments, the enucleated cell of the present disclosure comprises at least one therapeutic agent. In some embodiments, the enucleated cell of the present disclosure comprises at least two, three, four, five, six, seven, eight, nine, ten, or more therapeutic agents. In some embodiments, the therapeutic agent comprises an active agent. In some embodiments, the therapeutic agent is exogenous to the enucleated cell or parent cell thereof. An active agent comprises at least one of a DNA molecule, a RNA molecule, a protein (e.g., an enzyme, an antibody, an antigen, a toxin, cytokine, a protein hormone, a growth factor, a cell surface receptor, or a vaccine), a peptide (e.g., a peptide hormone or an antigen), a small molecule (e.g., a steroid, a polyketide, an alkaloid, a toxin, an antibiotic, an antiviral, a colchicine, a taxol, a mitomycin, or emtansine), a gene editing factor, a nanoparticle, or another active agent (e.g., bacteria, bacterial spores, bacteriophages, bacterial components, viruses (e.g., oncolytic viruses), exosomes, lipids, or ions). In some embodiments, the active agent is a cytokine, a growth factor, a hormone, an enzyme, a small molecule, a compound, or any combination thereof. In some embodiments, an enucleated cell is engineered to produce (e.g., express, and in some cases, release or secrete) the therapeutic agent. In some embodiments, the parent may be engineered to produce the therapeutic agent prior to enucleation to produce the enucleated cell. Non-limiting examples of oncolytic viruses include Talimogene laherparepvec, Onyx-015, GL-ONC1, CV706, Voyager-V1, and HSV-1716. Some wild-type viruses also show oncolytic behavior, such as Vaccinia virus, Vesicular stomatitis virus, Poliovirus, Reovirus, Senecavirus, ECHO-7, and Semliki Forest virus.

The therapeutic agent may be, or include, a targeting moiety described herein. Non-limiting example of the targeting moieties that may be produced by or contained in an enucleated cell includes chemokine receptors, adhesion molecules, and antigens. In some embodiments, the therapeutic agent may be, or include, a transmembrane moiety described herein.

In some embodiments, the therapeutic agent is recombinantly expressed by the enucleated cell or parent cell thereof. In some embodiments, the parent cell from which the enucleated cell is derived or obtained is engineered to produce or express the therapeutic agent. In some embodiments, expression of the therapeutic agent is stable (e.g., permanent). In some embodiments, the expression of the therapeutic agent by the parent cell is transient (e.g., non-permanent). In some embodiments, the parent cell is enucleated prior to engineering the enucleated cell to recombinantly express the therapeutic agent.

In some embodiments, the therapeutic agent is not naturally expressed (e.g., in the absence of engineering) in the cell from which the enucleated cell was derived or obtained (e.g., the therapeutic agent is exogenous to the parent cell). In some embodiments, the therapeutic agent is not naturally expressed in the subject (e.g., the therapeutic agent is exogenous to the subject). In some embodiments, the therapeutic agent is not naturally expressed in the subject at the intended site of therapy (e.g., a tumor, or a particular tissue, such as the brain, the intestine, the lungs, the heart, the liver, the spleen, the pancreas, muscles, eyes, and the like) (e.g., the therapeutic agent is exogenous to the intended site of therapy). In some embodiments, the level of the therapeutic agent is not naturally occurring in the enucleated cell of the parent cell.

In some embodiments, the therapeutic agent is naturally expressed (e.g., in the absence of engineering) in the cell from which the enucleated cell was derived or obtained (e.g., the therapeutic agent is endogenous to the enucleated cell). In some embodiments, the therapeutic agent is naturally expressed in the subject (e.g., the therapeutic agent is endogenous to the subject). In some embodiments, therapeutic agent is naturally expressed in the subject at the intended site of therapy (e.g., a tumor, or a particular tissue, such as the brain, the intestine, the lungs, the heart, the liver, the spleen, the pancreas, muscles, eyes, and the like) (e.g., the therapeutic agent is endogenous to the intended site of therapy).

In some embodiments, the therapeutic agent is derived from a synthetic cell and loaded into the enucleated cell. For example, the therapeutic agent may be endocytosed into the cell. Alternatively, the therapeutic agent may be synthesized by the cell and subsequently delivered to a target cell.

In some embodiments, the therapeutic agent comprises a corrected, a truncated, or a non-mutated version and/or copy of the DNA molecule, the RNA molecule, the protein, the peptide, the small molecule active agent, and/or the gene-editing factor as compared to the cell from which the enucleated cell was derived or obtained. For example, the therapeutic agent can correct a mutated p53 or EGFR in the target cell as part of the treatment for lung cancer.

In some embodiments, therapeutic agent comprises at least 2 (e.g., at least 2, 3, 4, 5, or more) different therapeutic DNA molecules, therapeutic RNA molecules, therapeutic proteins, therapeutic peptides, small molecule active agents, or therapeutic gene-editing factors, in any combination. For example, in some embodiments, a therapeutic agent comprises a therapeutic DNA molecule and a small molecule active agent. For example, in some embodiments, the therapeutic agent comprises two different small molecule active agents. For example, in some embodiments, the therapeutic agent comprises a chemokine receptor (e.g., for targeting) and a small molecule active agent.

In some embodiments, the therapeutic agent comprises an RNA molecule comprising messenger RNA (mRNA), short hairpin RNA (shRNA), small interfering RNA (siRNA), microRNA, long non-coding RNA (lncRNA) or an RNA virus. In some embodiments, the therapeutic agent comprises a DNA molecule that is single-stranded DNA, double-stranded DNA, an oligonucleotide, a plasmid, a bacterial DNA molecule or a DNA virus. In some embodiments, the therapeutic agent comprises a protein, or a portion thereof. In some embodiments, the protein is a cytokine, a growth factor, a hormone, an antibody or an antigen-binding fragment thereof, a small-peptide based drug, or an enzyme. In some embodiments, the enucleated cell transiently expresses the therapeutic agent. In some embodiments, the expression of the therapeutic agent is inducible. In some embodiments, the expression of the therapeutic agent permanent.

In some embodiments, the therapeutic agent comprises an exogenous agent. In some embodiments, the exogenous agent is an exogenous polypeptide. In some embodiments, the exogenous polypeptide is encoded by an exogenous polynucleotide delivered into the parent cell or the enucleated cell. In some embodiments, the exogenous polypeptide is synthesized or released by at least one intracellular organelle of the enucleated cell. In some embodiments, the exogenous polypeptide is released by the enucleated cell. In some embodiments, the exogenous polypeptide is expressed on the cell surface or the enucleated cell. In some embodiments, the enucleated cell delivers the exogenous polypeptide to a target cell. In some embodiments, the target cell is a cancer cell expressing the cancer biomarker of any cancer described herein. In some embodiments, the target cell is a pneumocyte. In some embodiments, the target cell is an epithelial cell. In some embodiments, the epithelial cells are located on, in, or from lung tissue. In some embodiments, the target cell is an endothelial cell expressing an endothelial biomarker described herein. In some embodiments, the endothelial cell is a blood vessel cell. In some embodiments, the endothelial cell is a lymphatic vessel cell.

In some embodiments, the exogenous polypeptide comprises a cytokine of any one of the cytokine described herein. In some embodiments, the exogenous polypeptide comprises a soluble cytokine. For example, the exogenous polypeptide can comprise an extracellular domain or fragment of the cytokine. In some embodiments, the exogenous polypeptide comprises a solubility as determined by turbidimetric solubility assay or thermodynamic solubility assay by dissolving the exogenous polypeptide in solvent such as organic solvent, including dimethyl sulfoxide (DMSO), dimethylformamide (DMF), acetonitrile, etc., or inorganic solvent, including water or phosphate-buffered saline (PBS). In some embodiments, the exogenous polypeptide comprises a solubility that is at least 0.0001 mg/ml, 0.0005 mg/ml, 0.001 mg/ml, 0.005 mg/ml, 0.01 mg/ml, 0.05 mg/ml, 0.1 mg/ml, 0.5 mg/ml, 1.0 mg/ml, 5.0 mg/ml, 10 mg/ml, 50 mg/ml, 100 mg/ml, 500 mg/ml 1,000 mg/ml 5,000 mg/ml, 10,000 mg/ml, 50,000 mg/ml, or 100,000 mg/ml.

In some embodiments, the exogenous polypeptide comprises a tumor necrosis factor (TNF) superfamily member or a catalytically active fragment thereof. Non-limiting examples of the TNF superfamily member include Lymphotoxin alpha (TNFβ), Tumor necrosis factor (TNFα), Lymphotoxin beta (TNFγ), OX40 ligand (CD252, Gp34, or CD134L), CD40 ligand (CD154, TRAP, Gp39, or T-BAM), Fas ligand (CD178, APTL, or CD95L), CD27 ligand (CD70), CD30 ligand (CD153), CD137 ligand (4-1 BBL), TNF-related apoptosis-inducing ligand (CD253 or APO-2L), Receptor activator of nuclear factor kappa-B ligand (CD254, OPGL, TRANCE, or ODF), TNF-related weak inducer of apoptosis (APO-3L or DR3L), a proliferation-inducing ligand (CD256, TALL-2, or TRDL1), B-cell activating factor (CD257, BLyS, TALL-1, or TNFSF20), LIGHT (CD258 or HVEML), Vascular endothelial growth inhibitor (TL1 or TL-1A), TNF superfamily member 18 (GITRL, AITRL, or TL-6), or Ectodysplasin A (ED1-A1 or ED1-A2).

In some embodiments, the therapeutic agent comprises any one of the immune checkpoint proteins described herein or an immune checkpoint inhibitor for inhibiting any one of the immune checkpoint protein described herein. Non-limiting examples of the immune checkpoint protein include PD-1, PD-L1, CTLA-4, VISTA, B7-H3 (also called CD276), A2AR, CD27, LAG3, TIM-3, T cell immunoreceptor with Ig and ITIM domains (TIGIT), CD73, NKG2A, PVRIG, PVRL2, CEACAMI, CEACAM5, CEACAM6, FAK, CCR-2, CCL-2, LIF, CD47, SIRPα, M-CSF, CSF-1R, IL-3, IL-1RAP, IL-8, SEMA4D, Angiopoietin-2, CLEVER-1, Axl, phosphatidylserine or a fragment thereof.

In some embodiments, the enucleated cells comprise an additional therapeutic agent, such as those disclosed herein. In some embodiments, the composition comprising the enucleated cells is formulated for administration to a subject disclosed herein with an additional therapeutic agent. In some embodiments, the additional therapeutic agent is administered to the subject sequentially, simultaneously, substantially sequentially, or substantially simultaneously.

### Pharmaceutical Compositions

Disclosed herein, in some aspects, are pharmaceutical compositions comprising the compositions disclosed herein and a pharmaceutically acceptable: carrier, excipient, diluent, or nebulized inhalant. In some embodiments, the compositions disclosed herein comprise the population of cells disclosed herein. In some embodiments, the compositions disclosed herein comprise one or more active agents or therapeutic agents. In some embodiments, the pharmaceutical composition disclosed herein comprise at least one additional active agent. In some embodiments, the pharmaceutical compositions disclosed herein comprise two or more active or therapeutic agents.

In some embodiments, the pharmaceutical composition is in a unit dose form. In some embodiments, the compositions comprise two or more active agents, or two or more therapeutic agents as disclosed herein. In some embodiments, the two or more active agents are contained in a single dosage unit, such as for example, when the enucleated cell comprises two or more therapeutic agents. In embodiments, the two or more active agents are contained in separate dosage units, such as when the enucleated cell is administered separately from an additional therapeutic agent or adjuvant. In some embodiments, the pharmaceutical composition described herein includes at least one additional active agent other than the enucleated cell described herein. In some embodiments, the at least one additional active agent is a cytokine, a growth factor, a hormone, an enzyme, a small molecule, a compound, or any combination thereof. In some embodiments, the at least one additional active agent is a chemotherapeutic agent, cytotoxic agent, cytokine, growth-inhibitory agent, anti-hormonal agent, anti-angiogenic agent, cardio protectant, and/or checkpoint inhibitor. Non-limiting checkpoint inhibitor includes IMP321/Eftilagimod alpha (Immutep), Relatlimab BMS-986016, Ipilimumab (Yervoy), Pembrolizumab (Keytruda), Nivolumab (Opdivo), Cemiplimab (Libtayo), Atezolizumab (Tecentriq), Avelumab (Bavencio), Durvalumab (Imfinzi), Ipilimumab (Yervoy), LAG525, MK-4280, Irinotecan, Oxaliplatin, REGN3767, TSR-033, B1754111, Sym022, FS118 (a bi-specific anti-LAG3/PD-L1 antagonistic mAb), MGD013 (a bi-specific anti-LAG3/PD-1 antagonistic mAb), TSR-022, Niraparib, Bevacizumab, MBG453, Decitabine, Spartalizumab, Sym023, INCAGN2390, LY3321367, Ramucirumab, Abemaciclib, Merestinib, BMS-986258, SHR-1702, Camrelizumab, MK-7684, Etigilimab/OMP-313 M32, Tiragolumab/MTIG7192A/RG-6058, BMS-986207, AB-154, ASP-8374, JNJ-61610588, CA-170d, Enoblituzumab/MGA271, MGD009, I-8H9/omburtamab, Trastuzumab, MGD013 (Anti-PD-1, anti-LAG-3 dual checkpoint inhibitor), BGB-A1217, CM-24 (MK-6018), BMS 986178, MEDI6469, PF-04518600, GSK3174998, MOXR0916, Utomilimab (PF-05082566), Urelumab (BMS-663513) ES101, BMS-986156, TRX-518, AMG 228, JTX-2011, GSK3359609, BMS-986226, MEDI-570, or Varlilumab (CDX-1127). Such compounds or drugs may be present in combination in amounts that are effective for the purpose intended. Additional non-limiting examples of the additional therapeutic agent include CPI-006 (for inhibiting CD73 and allowing T cell and APC activation); Monalizumab (for inhibiting NKG2A); COM701 (for inhibiting PVRIG/PVRL2 and activating T cell); CM24 (for inhibiting CEACAMI and allowing T and NK cells activation); NEO-201 (for inhibiting CEACAM5 and CEACAM6 which allows T cell activation while interfering with tumor cell growth); Defactinib (for inhibiting FAK and interfering with tumor growth); PF-04136309 (for inhibiting CCR-2 and CCL-2 and allowing T cell recruitment and activation); MSC-1 (for inhibiting LIF and allowing T cell and APC activation while interfering with cancer growth); Hu5F9-G4 (5F9), ALX148, TTI-662, and RRx-001 (for inhibiting CD47 or SIRPα and allowing T cell and APC activation); Lacnotuzumab (MCS-110), LY3022855, SNDX-6352, Emactuzumab (RG7155), and Pexidartinib (PLX3397) (for inhibiting M-CSF or CSF-1R and allowing APC activation); CAN04 and Canakinumab (ACZ885) (for inhibiting IL-3 or IL-1RAP and allowing T cell and APC activation); BMS-986253 (for inhibiting IL-8 and decreasing immunosuppressive tumor microenvironment while interfering with tumor growth); Pepinemab (VX15/2503) (for inhibiting SEMA4D and decreasing immunosuppressive tumor microenvironment while interfering with tumor growth); Trebananib (for inhibiting Angiopoietin-2 and allowing APC activation while interfering with cancer growth); FP-1305 (for inhibiting CLEVER-1 and allowing APC activation); Enapotamab vedotin (EnaV) (for inhibiting Axl and allowing APC activation while interfering with cancer growth); or Bavituximab (for inhibiting phosphatidylserine and allowing T cell and APC activation while interfering with cancer growth).

The compositions may include at least an exogenous therapeutic agent as an active ingredient in free-acid or free-base form, or in a pharmaceutically acceptable salt form. In addition, the methods and compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, compositions described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, I about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

### Formulations for Administration

The compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the compositions (e.g., pharmaceutical compositions disclosed herein) are formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or any combination thereof, to a subject. In some embodiments, the compositions (e.g., pharmaceutical compositions disclosed herein) are formulated for administering intravenously. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

For intravenous injections or drips or infusions, a composition described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Compositions for injection may be presented in unit dosage form, e.g., in ampoules or in multi dose containers, with an added preservative. The composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or nebulizers, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch. Formulations that include a composition are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compositions described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, the compositions of the exogenous therapeutic agents are in the form of a capsules, including push fit capsules made of gelatin, as well as soft, sealed capsules made of gelatin and a plasticizer, such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

Compositions for oral administration are in dosages suitable for such administration. In one aspect, solid oral dosage forms are prepared by mixing a composition with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the composition is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating may provide a delayed release of a therapeutic agent from the formulation. In other embodiments, the film coating aids in patient compliance. Film coatings may range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings.

In another aspect, dosage forms include microencapsulated formulations. In some embodiments, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the compositions described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

The compositions (e.g., pharmaceutical compositions) described herein may be formulated for administration to a subject by administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended-release formulations, pulsatile release formulations, multiparticulate formulations, and mixed immediate and controlled release formulations.

Buccal formulations are administered using a variety of formulations known in the art. In addition, the buccal dosage forms described herein may further include a bioerodible (hydrolysable) polymeric carrier that also serves to adhere the dosage form to the buccal mucosa. For buccal or sublingual administration, the compositions may take the form of tablets, lozenges, or gels formulated in a suitable manner.

For intravenous injections, a composition is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a composition described herein is in a form suitable for parenteral injection as a sterile suspensions, solutions or emulsions in oily or aqueous vehicles, and contain formulatory agents such as suspending, stabilizing and/or dispersing agents. The compositions for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Suitable formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., Wurster coating), tangential coating, top spraying, tableting, extruding and the like.

In some embodiments, the compositions are provided that include particles of a therapeutic agent and at least one dispersing agent or suspending agent for oral administration to a subject. The formulations may be a powder and/or granule for suspension, and upon admixture with water, a substantially uniform suspension is obtained.

Furthermore, the compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other the compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state for at least 4 hours. In one embodiment, an aqueous suspension is resuspended into a homogenous suspension by physical agitation lasting less than 1 minute. In still another embodiment, no agitation is necessary to maintain a homogeneous aqueous dispersion.

An aerosol formulation for nasal administration is generally an aqueous solution designed to be administered to the nasal passages in drops or sprays. Nasal solutions may be similar to nasal secretions in that they are generally isotonic and slightly buffered to maintain a pH of about 5.5 to about 6.5, although pH values outside of this range may additionally be used. Antimicrobial agents or preservatives may also be included in the formulation.

An aerosol formulation for inhalations and inhalants may be designed so that the agent or combination of agents is carried into the respiratory tree of the subject when administered by the nasal or oral respiratory route. Inhalation solutions may be administered, for example, by a nebulizer. Inhalations or insufflations, comprising finely powdered or liquid drugs, may be delivered to the respiratory system as a pharmaceutical aerosol of a solution or suspension of the agent or combination of agents in a propellant, e.g., to aid in disbursement. Propellants may be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Aerosol formulations may also include other components, for example, ethanol, isopropanol, propylene glycol, as well as surfactants or other components such as oils and detergents. These components may serve to stabilize the formulation and/or lubricate valve components.

The aerosol formulation may be packaged under pressure and may be formulated as an aerosol using solutions, suspensions, emulsions, powders and semisolid preparations. For example, a solution aerosol formulation comprises a solution of an agent such as a transporter, carrier, or ion channel inhibitor in (substantially) pure propellant or as a mixture of propellant and solvent. The solvent may be used to dissolve the agent and/or retard the evaporation of the propellant. Solvents may include, for example, water, ethanol and glycols. Any combination of suitable solvents may be use, optionally combined with preservatives, antioxidants, and/or other aerosol components.

An aerosol formulation may be a dispersion or suspension. A suspension aerosol formulation comprises a suspension of an agent or combination of agents, e.g., a transporter, carrier, or ion channel inhibitor, and a dispersing agent. Dispersing agents may include, for example, sorbitan trioleate, oleyl alcohol, oleic acid, lecithin and corn oil. A suspension aerosol formulation may also include lubricants, preservatives, antioxidant, and/or other aerosol components.

An aerosol formulation may similarly be formulated as an emulsion. An emulsion aerosol formulation may include, for example, an alcohol such as ethanol, a surfactant, water and a propellant, as well as an agent or combination of agents, e.g., a transporter, carrier, or ion channel. The surfactant used may be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

### Methods

Disclosed herein, in some embodiments, are methods of producing or using the compositions disclosed herein. In some embodiments, methods comprise high throughput techniques for enucleated cells to produce compositions comprising enucleated cells for biomedical applications with minimal residual nucleated parent cells. The methods disclosed herein also provide methods of using the enucleated cells as fusion partners (e.g., fusion to another cell in vivo or ex vivo), or a therapeutic agent delivery vehicle, or a combination thereof.

Disclosed herein, in some aspects, are methods of producing an enucleated cell described herein comprising enucleating a nucleated parent cell. In some embodiments, the parent cell may be treated with an exogenous molecule to soften cytoskeleton of the parent cell. For example, the parent cell can be treated with cytochalasin to soften the cortical actin cytoskeleton. In some embodiments, the nucleus is physically extracted from the cell body by centrifugation to generate an enucleated cell. In some embodiments, the centrifugation comprises use of density gradients, where the enucleated cells are isolated at least because the enucleate cells and intact nucleated cells sediment to different layers in the density gradient. In some embodiments, the density gradient can be constructed by material such as salts of alkali metals (e.g., cesium chloride, sodium iodide, sodium bromide, cesium sulfate, cesium acetate, or potassium tartrate; neutral and water-soluble molecules (e.g., saccharide); hydrophilic macromolecules (e.g., dextran) or synthetic molecules (e.g., sodium or methyl glucamine salt of triiodobenzoic acid and of metrizoic acid). In some embodiments, the density gradient is not comprised of colloid such as Percoll. In some embodiments, the density gradient comprises polysaccharide such as Ficoll.

In some embodiments, the centrifugation comprises fixed angle centrifugation, including continuous flow centrifugation. Example 1 illustrates an exemplary fixed angle centrifugation experiment for obtaining enucleated cells from nucleated cells. In some embodiments, the fixed angle centrifugation comprises ultracentrifugation. In some embodiments, the fixed angle centrifugation comprises continuous-flow centrifugation (e.g., fixed angle centrifugation by Sorvall CC40NX centrifugation). In some embodiments, the use of fixed angle centrifugation increases the volume that can be centrifuged. For example, the use of fixed angle centrifugation increases a volume that can be centrifuged compared to swinging-bucket centrifugation (for generating a comparable density gradient). In some embodiments, the use of fixed angle rotors can hold a greater quantity of tubes compared to its swinging-bucket counterpart. In some embodiments, the fixed angle rotors can withstand much higher gravitational forces, and thus, shorter centrifugation times. In some embodiments, a combination of high centrifugal force and short centrifugation makes fixed angle centrifugation useful for a variety of applications from pelleting bacteria and cells to isopycnic separation of macromolecules. In some embodiments, the nucleated cells can be engineered to possess a heterologous polynucleotide encoding a heterologous gene product described herein.

In some embodiments, methods disclosed herein result in a composition comprising tens of millions of enucleated cells (“enucleated cell fraction”). In some embodiments, the composition also includes residual nucleated cells (“nucleated cell fraction”). In some embodiments, the composition is further processed to purify the enucleated cell fraction from the nucleated cell fraction. In some embodiments, the enucleated cell fraction is formulated in a pharmaceutical composition comprising a pharmaceutically acceptable: carrier, excipient, or diluent.

In some embodiments, methods of producing the enucleated cell do not consist or comprise of differentiation of the parent cell. For example, the enucleated cell is not obtained by differentiating a nucleated erythroid progenitor cell into a differentiated and enucleated red blood cell. In some embodiments, the enucleated cell is not a terminally differentiated cell. In some embodiments, the enucleated cell is not a platelet. In some embodiments, the enucleated cell is not obtained from a platelet lineage cell. In some embodiments, the enucleated cell is not a red blood cell. In some embodiments, the enucleated cell is not obtained from a red blood cell lineage cell.

In some embodiments, the parent cell containing a nucleus is engineered to express at least one of therapeutic agent, transmembrane moiety, immune-evading moiety, or targeting moiety described herein; and subsequently, the nucleus of the parent cell is removed. In some embodiments, the parent cell containing the nucleus is enucleated, and the enucleated cell is engineered to express therapeutic agent, transmembrane moiety, immune-evading moiety, or targeting moiety described herein. In some embodiments, the parent cell is engineered to express one or more of the biomolecules above (e.g., immune-evading moiety and/or targeting moiety), and the resulting enucleated cell (e.g., already expressing the immune-evading moiety and/or targeting moiety) is further engineered to express a second of the biomolecules above (e.g., a therapeutic agent). In this manner, the enucleated cells of the present disclosure can be extensively engineered prior to enucleation, stored for long periods of time as needed (through for e.g., lyophilization, cryohibernation, cryopreservation), and quickly engineered to express a therapeutic agent closer to the time of need.

In some embodiments, the composition disclosed herein is loaded in a tube for fixed angle centrifugation.

In some embodiments, the fixed angle centrifugation comprises at least one tube. In some embodiments, the fixed angle centrifugation comprises at least two tubes. In some embodiments, the fixed angle centrifugation comprises at least three tubes. In some embodiments, the fixed angle centrifugation comprises at least four tubes. In some embodiments, the fixed angle centrifugation comprises at least five tubes. In some embodiments, the fixed angle centrifugation comprises at least six tubes. In some embodiments, the fixed angle centrifugation comprises at least seven tubes. In some embodiments, the fixed angle centrifugation comprises at least eight tubes. In some embodiments, the fixed angle centrifugation comprises at least nine tubes. In some embodiments, the fixed angle centrifugation comprises at least ten tubes.

In some embodiments, the composition has the volume comprising ranging from about 0.001 L to about 10 L. In some embodiments, the composition has the volume comprising ranging from about 0.001 L to about 0.01 L, about 0.001 L to about 0.1 L, about 0.001 L to about 1 L, about 0.001 L to about 2 L, about 0.001 L to about 3 L, about 0.001 L to about 5 L, about 0.001 L to about 6 L, about 0.001 L to about 7 L, about 0.001 L to about 8 L, about 0.001 L to about 9 L, about 0.001 L to about 10 L, about 0.01 L to about 0.1 L, about 0.01 L to about 1 L, about 0.01 L to about 2 L, about 0.01 L to about 3 L, about 0.01 L to about 5 L, about 0.01 L to about 6 L, about 0.01 L to about 7 L, about 0.01 L to about 8 L, about 0.01 L to about 9 L, about 0.01 L to about 10 L, about 0.1 L to about 1 L, about 0.1 L to about 2 L, about 0.1 L to about 3 L, about 0.1 L to about 5 L, about 0.1 L to about 6 L, about 0.1 L to about 7 L, about 0.1 L to about 8 L, about 0.1 L to about 9 L, about 0.1 L to about 10 L, about 1 L to about 2 L, about 1 L to about 3 L, about 1 L to about 5 L, about 1 L to about 6 L, about 1 L to about 7 L, about 1 L to about 8 L, about 1 L to about 9 L, about 1 L to about 10 L, about 2 L to about 3 L, about 2 L to about 5 L, about 2 L to about 6 L, about 2 L to about 7 L, about 2 L to about 8 L, about 2 L to about 9 L, about 2 L to about 10 L, about 3 L to about 5 L, about 3 L to about 6 L, about 3 L to about 7 L, about 3 L to about 8 L, about 3 L to about 9 L, about 3 L to about 10 L, about 5 L to about 6 L, about 5 L to about 7 L, about 5 L to about 8 L, about 5 L to about 9 L, about 5 L to about 10 L, about 6 L to about 7 L, about 6 L to about 8 L, about 6 L to about 9 L, about 6 L to about 10 L, about 7 L to about 8 L, about 7 L to about 9 L, about 7 L to about 10 L, about 8 L to about 9 L, about 8 L to about 10 L, or about 9 L to about 10 L. In some embodiments, the composition has the volume comprising ranging from about 0.001 L, about 0.01 L, about 0.1 L, about 1 L, about 2 L, about 3 L, about 5 L, about 6 L, about 7 L, about 8 L, about 9 L, or about 10 L. In some embodiments, the composition has the volume comprising ranging from at least about 0.001 L, about 0.01 L, about 0.1 L, about 1 L, about 2 L, about 3 L, about 5 L, about 6 L, about 7 L, about 8 L, or about 9 L. In some embodiments, the composition has the volume comprising ranging from at most about 0.01 L, about 0.1 L, about 1 L, about 2 L, about 3 L, about 5 L, about 6 L, about 7 L, about 8 L, about 9 L, or about 10 L.

In some embodiments, described herein are methods for cell processing by enucleating a portion of the nucleated cells (parent cells) to produce an enucleated cell fraction using fixed angle centrifugation, including continuous flow centrifugation.

In some embodiments, the resulting composition comprises an enucleated cell fraction, which may be 100% of the composition. In other embodiments, there may be a nucleated cell fraction of the composition comprised of nucleated parent cells that were not enucleated. In some embodiments, the enucleated cell fraction is greater than or equal and about 30% composition. In some embodiments, the enucleated cell fraction is greater than or equal to about 35% of the composition. In some embodiments, the enucleated cell fraction is greater than or equal to about 40% of the composition. In some embodiments, the enucleated cell fraction is greater than or equal to about 45% of the composition. In some embodiments, the enucleated cell fraction is greater than or equal to about 50% of the composition. In some embodiments, the enucleated cell fraction is greater than or equal to about 55% of the composition. In some embodiments, the enucleated cell fraction is greater than or equal to about 60% of the composition. In some embodiments, the enucleated cell fraction is greater than or equal to about 65% of the composition. In some embodiments, the enucleated cell fraction is greater than or equal to about 70% of the composition. In some embodiments, the enucleated cell fraction is greater than or equal to about 75% of the composition. In some embodiments, the enucleated cell fraction is greater than or equal to about 80% of the composition. In some embodiments, the enucleated cell fraction is greater than or equal to about 85% of the composition. In some embodiments, the enucleated cell fraction is greater than or equal to about 90% of the composition. In some embodiments, the enucleated cell fraction is greater than or equal to about 95% of the composition. In some embodiments, the enucleated cell fraction is greater than or equal to about 96% of the composition. In some embodiments, the enucleated cell fraction is greater than or equal to about 97% of the composition. In some embodiments, the enucleated cell fraction is greater than or equal to about 98% of the composition. In some embodiments, the enucleated cell fraction is greater than or equal to about 99% of the composition.

In some embodiments, cell separation, cell isolation, or cell sorting, is a process to isolate one or more specific cell populations from a heterogeneous mixture of cells. In some embodiments, the methods of enucleating cells disclosed herein is performed on an isolated population of homogenous cells. In some embodiments, the methods of enucleated cells disclosed herein is performed on a heterogeneous mixture of cells. In some embodiments, methods disclosed herein comprise isolating a population of homogenous cells from a mixture of heterogenous cells using a suitable cell separation technique including, but not limited to, immunomagnetic cell separation, fluorescence-activated cell sorting, density gradient centrifugation, immune density cell isolation, microfluidic cell sorting, buoyancy-activated cell sorting, aptamer-based cell isolation, complement depletion, or any combination thereof.

In centrifugation, more dense particles can move to the outer edges of the mixture while less dense objects groups together further in as the sample is spun. A biological sample can be centrifuged until the cell types are isolated into layers. During centrifugation, each cell type can sediment to its isopycnic point, which is the place in the medium gradient, where the density of the cells and medium are equal. Examples of a particle density gradient media include Lymphoprep™, Lympholyte®, Ficoll-Paque®, Percoll®, OptiPrep™, Cell Separation with Accuspin™ Aystem-Histopaque® Media, Histopaque® Media, Histopaque® Iodinated Gradient Media, inorganic salts, nonionic iodinated density gradient media, polyhydric alcohols, polysaccharides, etc. For instance, Lymphoprep™, Lympholyte®, and Ficoll-Paque® consists of saccharides and sodium diatrizoate and may be used to isolate mononuclear cells from peripheral blood, cord blood, and bone marrow. Percoll® consists of colloidal silica particles coated with polyvinylpyrrolidone and is widely used to separate cells, organelles, viruses, and other subcellular particles. OptiPrep™ is a medium consisting of iodixanol in water and used to isolate viruses, organelles, macromolecules, and cells. In some embodiments, the particle density gradient can be established by overlaying various concentrations of polyhydric (sugar) alcohols (e.g., sucrose, glycerol, or sorbitol), polysaccharides (e.g., Ficoll, polysucrose, or dextrans), inorganic salts (CsCl, Cs₂SO₄, or KBr), iodinated compounds (Diatrizoate, Nycodenz, Histodenz, or iodixanol), colloidal silica (e.g., Percoll), or a combination thereof. In some embodiments, the particle density gradient can be established by overlaying various concentrations of a peptide or a protein solution. For example, the particle density gradient can be established with serum albumin solutions.

In some embodiments, the less dense particles can move to the outer edges of the mixtures while the denser particles group together further in. For example, the use of the continuous flow centrifugation (e.g., fixed angle centrifugation by Sorvall CC40NX centrifugation) can establish such density gradient, where the loading of the particle gradient is done from lowest density to highest density starting from the bottom of the centrifuge. In some embodiments, the particle density gradient can be established by loading the less dense particle solution followed by dense particle solutions into the fixed angle centrifuge. In some embodiments, the particle density gradient can be established by loading the denser particle solution followed by less dense particle solutions into the fixed angle centrifuge.

In some embodiments, disclosed herein are methods for cell processing by enucleating a portion of the nucleated cells to produce an enucleated cell fraction using fixed angle centrifugation, including continuous flow centrifugation. In some embodiments, disclosed herein are methods for cell processing by enucleating a portion of the nucleated cells to produce an enucleated cell fraction using zonal centrifugation. In some embodiments, the fixed angle centrifugation is continuous flow centrifugation. In some embodiments, the fixed angle centrifugation generates a density gradient. In some embodiments, the density gradient separates the enucleated cell fraction from the nucleated cells in the composition. In some embodiments, the density gradient comprises a polysaccharide density gradient. In some embodiments, the polysaccharide density gradient comprises a Ficoll density gradient. In some embodiments, methods further comprise producing the Ficoll gradient by polymerizing sucrose molecules with epichlorohydrin to give a polysaccharide that is osmotically inert.

In some embodiments, the gradient comprises between 2 ranges to 20 ranges of the density gradient. In some embodiments, the gradient comprises between 2 ranges to 3 ranges, 2 ranges to 4 ranges, 2 ranges to 5 ranges, 2 ranges to 6 ranges, 2 ranges to 8 ranges, 2 ranges to 10 ranges, 2 ranges to 12 ranges, 2 ranges to 14 ranges, 2 ranges to 16 ranges, 2 ranges to 18 ranges, 2 ranges to 20 ranges, 3 ranges to 4 ranges, 3 ranges to 5 ranges, 3 ranges to 6 ranges, 3 ranges to 8 ranges, 3 ranges to 10 ranges, 3 ranges to 12 ranges, 3 ranges to 14 ranges, 3 ranges to 16 ranges, 3 ranges to 18 ranges, 3 ranges to 20 ranges, 4 ranges to 5 ranges, 4 ranges to 6 ranges, 4 ranges to 8 ranges, 4 ranges to 10 ranges, 4 ranges to 12 ranges, 4 ranges to 14 ranges, 4 ranges to 16 ranges, 4 ranges to 18 ranges, 4 ranges to 20 ranges, 5 ranges to 6 ranges, 5 ranges to 8 ranges, 5 ranges to 10 ranges, 5 ranges to 12 ranges, 5 ranges to 14 ranges, 5 ranges to 16 ranges, 5 ranges to 18 ranges, 5 ranges to 20 ranges, 6 ranges to 8 ranges, 6 ranges to 10 ranges, 6 ranges to 12 ranges, 6 ranges to 14 ranges, 6 ranges to 16 ranges, 6 ranges to 18 ranges, 6 ranges to 20 ranges, 8 ranges to 10 ranges, 8 ranges to 12 ranges, 8 ranges to 14 ranges, 8 ranges to 16 ranges, 8 ranges to 18 ranges, 8 ranges to 20 ranges, 10 ranges to 12 ranges, 10 ranges to 14 ranges, 10 ranges to 16 ranges, 10 ranges to 18 ranges, 10 ranges to 20 ranges, 12 ranges to 14 ranges, 12 ranges to 16 ranges, 12 ranges to 18 ranges, 12 ranges to 20 ranges, 14 ranges to 16 ranges, 14 ranges to 18 ranges, 14 ranges to 20 ranges, 16 ranges to 18 ranges, 16 ranges to 20 ranges, or 18 ranges to 20 ranges of the density gradient. In some embodiments, the gradient comprises between 2 ranges, 3 ranges, 4 ranges, 5 ranges, 6 ranges, 8 ranges, 10 ranges, 12 ranges, 14 ranges, 16 ranges, 18 ranges, or 20 ranges. In some embodiments, the gradient comprises between at least 2 ranges, 3 ranges, 4 ranges, 5 ranges, 6 ranges, 8 ranges, 10 ranges, 12 ranges, 14 ranges, 16 ranges, or 18 ranges of the density gradient. In some embodiments, the gradient comprises between at most 3 ranges, 4 ranges, 5 ranges, 6 ranges, 8 ranges, 10 ranges, 12 ranges, 14 ranges, 16 ranges, 18 ranges, or 20 ranges of the density gradient. In some embodiments, the gradient comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty ranges of the density gradient. In some embodiments, the gradient comprises at least seven ranges of the density gradient. In some embodiments, the gradient comprises at least five ranges of the density gradient. In some embodiments, the gradient comprises at least three ranges of the density gradient. In some embodiments, the gradient comprises seven ranges of the density gradient. In some embodiments, the gradient comprises five ranges of the density gradient. In some embodiments, the gradient comprises three ranges of the density gradient.

In some embodiments, the gradient comprises about 7.5% density gradient media to about 30% density gradient media. 7.5% density gradient media to about 10% density gradient media, about 7.5% density gradient media to about 12.5% density gradient media, about 7.5% density gradient media to about 15% density gradient media, about 7.5% density gradient media to about 16% density gradient media, about 7.5% density gradient media to about 17% density gradient media, about 7.5% density gradient media to about 18% density gradient media, about 7.5% density gradient media to about 19% density gradient media, about 7.5% density gradient media to about 20% density gradient media, about 7.5% density gradient media to about 25% density gradient media, about 7.5% density gradient media to about 27.5% density gradient media, about 7.5% density gradient media to about 30% density gradient media, about 10% density gradient media to about 12.5% density gradient media, about 10% density gradient media to about 15% density gradient media, about 10% density gradient media to about 16% density gradient media, about 10% density gradient media to about 17% density gradient media, about 10% density gradient media to about 18% density gradient media, about 10% density gradient media to about 19% density gradient media, about 10% density gradient media to about 20% density gradient media, about 10% density gradient media to about 25% density gradient media, about 10% density gradient media to about 27.5% density gradient media, about 10% density gradient media to about 30% density gradient media, about 12.5% density gradient media to about 15% density gradient media, about 12.5% density gradient media to about 16% density gradient media, about 12.5% density gradient media to about 17% density gradient media, about 12.5% density gradient media to about 18% density gradient media, about 12.5% density gradient media to about 19% density gradient media, about 12.5% density gradient media to about 20% density gradient media, about 12.5% density gradient media to about 25% density gradient media, about 12.5% density gradient media to about 27.5% density gradient media, about 12.5% density gradient media to about 30% density gradient media, about 15% density gradient media to about 16% density gradient media, about 15% density gradient media to about 17% density gradient media, about 15% density gradient media to about 18% density gradient media, about 15% density gradient media to about 19% density gradient media, about 15% density gradient media to about 20% density gradient media, about 15% density gradient media to about 25% density gradient media, about 15% density gradient media to about 27.5% density gradient media, about 15% density gradient media to about 30% density gradient media, about 16% density gradient media to about 17% density gradient media, about 16% density gradient media to about 18% density gradient media, about 16% density gradient media to about 19% density gradient media, about 16% density gradient media to about 20% density gradient media, about 16% density gradient media to about 25% density gradient media, about 16% density gradient media to about 27.5% density gradient media, about 16% density gradient media to about 30% density gradient media, about 17% density gradient media to about 18% density gradient media, about 17% density gradient media to about 19% density gradient media, about 17% density gradient media to about 20% density gradient media, about 17% density gradient media to about 25% density gradient media, about 17% density gradient media to about 27.5% density gradient media, about 17% density gradient media to about 30% density gradient media, about 18% density gradient media to about 19% density gradient media, about 18% density gradient media to about 20% density gradient media, about 18% density gradient media to about 25% density gradient media, about 18% density gradient media to about 27.5% density gradient media, about 18% density gradient media to about 30% density gradient media, about 19% density gradient media to about 20% density gradient media, about 19% density gradient media to about 25% density gradient media, about 19% density gradient media to about 27.5% density gradient media, about 19% density gradient media to about 30% density gradient media, about 20% density gradient media to about 25% density gradient media, about 20% density gradient media to about 27.5% density gradient media, about 20% density gradient media to about 30% density gradient media, about 25% density gradient media to about 27.5% density gradient media, about 25% density gradient media to about 30% density gradient media, or about 27.5% density gradient media to about 30% density gradient media.

In some embodiments, the gradient is a Ficoll gradient. In some embodiments, the Ficoll gradient comprises between 2 ranges to 20 ranges of the density Ficoll gradient. In some embodiments, the Ficoll gradient comprises between 2 ranges to 3 ranges, 2 ranges to 4 ranges, 2 ranges to 5 ranges, 2 ranges to 6 ranges, 2 ranges to 8 ranges, 2 ranges to 10 ranges, 2 ranges to 12 ranges, 2 ranges to 14 ranges, 2 ranges to 16 ranges, 2 ranges to 18 ranges, 2 ranges to 20 ranges, 3 ranges to 4 ranges, 3 ranges to 5 ranges, 3 ranges to 6 ranges, 3 ranges to 8 ranges, 3 ranges to 10 ranges, 3 ranges to 12 ranges, 3 ranges to 14 ranges, 3 ranges to 16 ranges, 3 ranges to 18 ranges, 3 ranges to 20 ranges, 4 ranges to 5 ranges, 4 ranges to 6 ranges, 4 ranges to 8 ranges, 4 ranges to 10 ranges, 4 ranges to 12 ranges, 4 ranges to 14 ranges, 4 ranges to 16 ranges, 4 ranges to 18 ranges, 4 ranges to 20 ranges, 5 ranges to 6 ranges, 5 ranges to 8 ranges, 5 ranges to 10 ranges, 5 ranges to 12 ranges, 5 ranges to 14 ranges, 5 ranges to 16 ranges, 5 ranges to 18 ranges, 5 ranges to 20 ranges, 6 ranges to 8 ranges, 6 ranges to 10 ranges, 6 ranges to 12 ranges, 6 ranges to 14 ranges, 6 ranges to 16 ranges, 6 ranges to 18 ranges, 6 ranges to 20 ranges, 8 ranges to 10 ranges, 8 ranges to 12 ranges, 8 ranges to 14 ranges, 8 ranges to 16 ranges, 8 ranges to 18 ranges, 8 ranges to 20 ranges, 10 ranges to 12 ranges, 10 ranges to 14 ranges, 10 ranges to 16 ranges, 10 ranges to 18 ranges, 10 ranges to 20 ranges, 12 ranges to 14 ranges, 12 ranges to 16 ranges, 12 ranges to 18 ranges, 12 ranges to 20 ranges, 14 ranges to 16 ranges, 14 ranges to 18 ranges, 14 ranges to 20 ranges, 16 ranges to 18 ranges, 16 ranges to 20 ranges, or 18 ranges to 20 ranges of the density Ficoll gradient. In some embodiments, the Ficoll gradient comprises between 2 ranges, 3 ranges, 4 ranges, 5 ranges, 6 ranges, 8 ranges, 10 ranges, 12 ranges, 14 ranges, 16 ranges, 18 ranges, or 20 ranges. In some embodiments, the Ficoll gradient comprises between at least 2 ranges, 3 ranges, 4 ranges, 5 ranges, 6 ranges, 8 ranges, 10 ranges, 12 ranges, 14 ranges, 16 ranges, or 18 ranges of the density Ficoll gradient. In some embodiments, the Ficoll gradient comprises between at most 3 ranges, 4 ranges, 5 ranges, 6 ranges, 8 ranges, 10 ranges, 12 ranges, 14 ranges, 16 ranges, 18 ranges, or 20 ranges of the density Ficoll gradient. In some embodiments, the Ficoll gradient comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty ranges of the density Ficoll gradient. In some embodiments, the Ficoll gradient comprises at least seven ranges of the density Ficoll gradient. In some embodiments, the Ficoll gradient comprises at least five ranges of the density Ficoll gradient. In some embodiments, the Ficoll gradient comprises at least three ranges of the density Ficoll gradient. In some embodiments, the Ficoll gradient comprises seven ranges of the density Ficoll gradient. In some embodiments, the Ficoll gradient comprises five ranges of the density Ficoll gradient. In some embodiments, the Ficoll gradient comprises three ranges of the density Ficoll gradient.

In some embodiments, the Ficoll density gradient comprises about 7.5% Ficoll to about 10% Ficoll, about 7.5% Ficoll to about 12.5% Ficoll, about 7.5% Ficoll to about 15% Ficoll, about 7.5% Ficoll to about 16% Ficoll, about 7.5% Ficoll to about 17% Ficoll, about 7.5% Ficoll to about 18% Ficoll, about 7.5% Ficoll to about 19% Ficoll, about 7.5% Ficoll to about 20% Ficoll, about 7.5% Ficoll to about 25% Ficoll, about 7.5% Ficoll to about 27.5% Ficoll, about 7.5% Ficoll to about 30% Ficoll, about 10% Ficoll to about 12.5% Ficoll, about 10% Ficoll to about 15% Ficoll, about 10% Ficoll to about 16% Ficoll, about 10% Ficoll to about 17% Ficoll, about 10% Ficoll to about 18% Ficoll, about 10% Ficoll to about 19% Ficoll, about 10% Ficoll to about 20% Ficoll, about 10% Ficoll to about 25% Ficoll, about 10% Ficoll to about 27.5% Ficoll, about 10% Ficoll to about 30% Ficoll, about 12.5% Ficoll to about 15% Ficoll, about 12.5% Ficoll to about 16% Ficoll, about 12.5% Ficoll to about 17% Ficoll, about 12.5% Ficoll to about 18% Ficoll, about 12.5% Ficoll to about 19% Ficoll, about 12.5% Ficoll to about 20% Ficoll, about 12.5% Ficoll to about 25% Ficoll, about 12.5% Ficoll to about 27.5% Ficoll, about 12.5% Ficoll to about 30% Ficoll, about 15% Ficoll to about 16% Ficoll, about 15% Ficoll to about 17% Ficoll, about 15% Ficoll to about 18% Ficoll, about 15% Ficoll to about 19% Ficoll, about 15% Ficoll to about 20% Ficoll, about 15% Ficoll to about 25% Ficoll, about 15% Ficoll to about 27.5% Ficoll, about 15% Ficoll to about 30% Ficoll, about 16% Ficoll to about 17% Ficoll, about 16% Ficoll to about 18% Ficoll, about 16% Ficoll to about 19% Ficoll, about 16% Ficoll to about 20% Ficoll, about 16% Ficoll to about 25% Ficoll, about 16% Ficoll to about 27.5% Ficoll, about 16% Ficoll to about 30% Ficoll, about 17% Ficoll to about 18% Ficoll, about 17% Ficoll to about 19% Ficoll, about 17% Ficoll to about 20% Ficoll, about 17% Ficoll to about 25% Ficoll, about 17% Ficoll to about 27.5% Ficoll, about 17% Ficoll to about 30% Ficoll, about 18% Ficoll to about 19% Ficoll, about 18% Ficoll to about 20% Ficoll, about 18% Ficoll to about 25% Ficoll, about 18% Ficoll to about 27.5% Ficoll, about 18% Ficoll to about 30% Ficoll, about 19% Ficoll to about 20% Ficoll, about 19% Ficoll to about 25% Ficoll, about 19% Ficoll to about 27.5% Ficoll, about 19% Ficoll to about 30% Ficoll, about 20% Ficoll to about 25% Ficoll, about 20% Ficoll to about 27.5% Ficoll, about 20% Ficoll to about 30% Ficoll, about 25% Ficoll to about 27.5% Ficoll, about 25% Ficoll to about 30% Ficoll, or about 27.5% Ficoll to about 30% Ficoll.

In some embodiments, the Ficoll density gradient comprises about 7.5% Ficoll, about 10% Ficoll, about 12.5% Ficoll, about 15% Ficoll, about 16% Ficoll, about 17% Ficoll, about 18% Ficoll, about 19% Ficoll, about 20% Ficoll, about 25% Ficoll, about 27.5% Ficoll, or about 30% Ficoll. In some embodiments, the Ficoll density gradient comprises at least about 7.5% Ficoll, about 10% Ficoll, about 12.5% Ficoll, about 15% Ficoll, about 16% Ficoll, about 17% Ficoll, about 18% Ficoll, about 19% Ficoll, about 20% Ficoll, about 25% Ficoll, or about 27.5% Ficoll. In some embodiments, the Ficoll density gradient comprises at most about 10% Ficoll, about 12.5% Ficoll, about 15% Ficoll, about 16% Ficoll, about 17% Ficoll, about 18% Ficoll, about 19% Ficoll, about 20% Ficoll, about 25% Ficoll, about 27.5% Ficoll, or about 30% Ficoll. In some embodiments, the Ficoll density gradient comprises about 25% Ficoll, about 17% Ficoll, about 16% Ficoll, about 15% Ficoll, or about 12.5% Ficoll. In some embodiments, the Ficoll density gradient comprises about 25% Ficoll. In some embodiments, the Ficoll density gradient comprises about 17% Ficoll. In some embodiments, the Ficoll density gradient comprises about 16% Ficoll. In some embodiments, the Ficoll density gradient comprises about 15% Ficoll. In some embodiments, the Ficoll density gradient comprises about 12.5% Ficoll. In some embodiments, the Ficoll percentage is synonymous with the polysaccharide solution.

In some embodiments, the fixed angle centrifugation comprises centrifugating the plurality of cells in the polysaccharide density gradient, relative to the axis of rotation, at a 20-degree angle, at a 21-degree angle, at a 22-degree angle, at a 23-degree angle, at a 24-degree angle, at a 25-degree angle, at a 26-degree angle, at a 27-degree angle, at a 28-degree angle, at a 29-degree angle, at a 30-degree angle, at a 31-degree angle, at a 32-degree angle, at a 33-degree angle, at a 34-degree angle, at a 35-degree angle, at a 36-degree angle, at a 37-degree angle, at a 38-degree angle, at a 39-degree angle, at a 40-degree angle, at a 41-degree angle, at a 42-degree angle, at a 43-degree angle, at a 44-degree angle, at a 45-degree angle, at a 46-degree angle, at a 47-degree angle, at a 48-degree angle, at a 49-degree angle, at a 50-degree angle, at a 90-degree angle, or at a 180-degree angle. In some embodiments, the fixed angle centrifugation comprises centrifugating the plurality of cells in the polysaccharide density gradient at a 23-degree angle, at a 24-degree angle, at a 25-degree angle, or at a 45-degree angle. In some embodiments, the fixed angle centrifugation comprises centrifugating the plurality of cells in the polysaccharide density gradient at a 23-degree angle. In some embodiments, the fixed angle centrifugation comprises centrifugating the plurality of cells in the polysaccharide density gradient at a 24-degree angle. In some embodiments, the fixed angle centrifugation comprises centrifugating the plurality of cells in the polysaccharide density gradient at a 25-degree angle. In some embodiments, the fixed angle centrifugation comprises centrifugating the plurality of cells in the polysaccharide density gradient at a 45-degree angle. In some embodiments, the fixed angle centrifugation comprises centrifugating the plurality of cells in the polysaccharide density gradient at a 90-degree angle. In some embodiments, the fixed angle centrifugation comprises centrifugating the plurality of cells in the polysaccharide density gradient at a 180-degree angle.

In some embodiments, the fixed angle centrifugation is generated by a fixed angle rotor. In some embodiments, the fixed angle centrifugation is generated by a combination of fixed angle rotor and a swinging bucket rotor. For example, the cells and density gradient can be loaded in a vertical position (0 degree) and, when it spins, it swings out to a fixed angle (e.g., 25 or 45 degree angle). In some embodiments, the fixed angle centrifugation is not generated by swinging bucket centrifugation.

In some embodiments, the methods of enucleating a portion of the nucleated cells to produce an enucleated cell fraction using fixed angle centrifugation for cell processing further include generating the density gradient by centrifuging a polysaccharide.

In some embodiments, the fixed angle centrifugation comprises centrifugation between about 10,000 Relative Centrifugal Force (RCF) to about 250,000 RCF. In some embodiments, the fixed angle centrifugation comprises centrifugation between about 10,000 RCF to about 20,000 RCF, about 10,000 RCF to about 30,000 RCF, about 10,000 RCF to about 40,000 RCF, about 10,000 RCF to about 50,000 RCF, about 10,000 RCF to about 60,000 RCF, about 10,000 RCF to about 70,000 RCF, about 10,000 RCF to about 80,000 RCF, about 10,000 RCF to about 100,000 RCF, about 10,000 RCF to about 150,000 RCF, about 10,000 RCF to about 200,000 RCF, about 10,000 RCF to about 250,000 RCF, about 20,000 RCF to about 30,000 RCF, about 20,000 RCF to about 40,000 RCF, about 20,000 RCF to about 50,000 RCF, about 20,000 RCF to about 60,000 RCF, about 20,000 RCF to about 70,000 RCF, about 20,000 RCF to about 80,000 RCF, about 20,000 RCF to about 100,000 RCF, about 20,000 RCF to about 150,000 RCF, about 20,000 RCF to about 200,000 RCF, about 20,000 RCF to about 250,000 RCF, about 30,000 RCF to about 40,000 RCF, about 30,000 RCF to about 50,000 RCF, about 30,000 RCF to about 60,000 RCF, about 30,000 RCF to about 70,000 RCF, about 30,000 RCF to about 80,000 RCF, about 30,000 RCF to about 100,000 RCF, about 30,000 RCF to about 150,000 RCF, about 30,000 RCF to about 200,000 RCF, about 30,000 RCF to about 250,000 RCF, about 40,000 RCF to about 50,000 RCF, about 40,000 RCF to about 60,000 RCF, about 40,000 RCF to about 70,000 RCF, about 40,000 RCF to about 80,000 RCF, about 40,000 RCF to about 100,000 RCF, about 40,000 RCF to about 150,000 RCF, about 40,000 RCF to about 200,000 RCF, about 40,000 RCF to about 250,000 RCF, about 50,000 RCF to about 60,000 RCF, about 50,000 RCF to about 70,000 RCF, about 50,000 RCF to about 80,000 RCF, about 50,000 RCF to about 100,000 RCF, about 50,000 RCF to about 150,000 RCF, about 50,000 RCF to about 200,000 RCF, about 50,000 RCF to about 250,000 RCF, about 60,000 RCF to about 70,000 RCF, about 60,000 RCF to about 80,000 RCF, about 60,000 RCF to about 100,000 RCF, about 60,000 RCF to about 150,000 RCF, about 60,000 RCF to about 200,000 RCF, about 60,000 RCF to about 250,000 RCF, about 70,000 RCF to about 80,000 RCF, about 70,000 RCF to about 100,000 RCF, about 70,000 RCF to about 150,000 RCF, about 70,000 RCF to about 200,000 RCF, about 70,000 RCF to about 250,000 RCF, about 80,000 RCF to about 100,000 RCF, about 80,000 RCF to about 150,000 RCF, about 80,000 RCF to about 200,000 RCF, about 80,000 RCF to about 250,000 RCF, about 100,000 RCF to about 150,000 RCF, about 100,000 RCF to about 200,000 RCF, about 100,000 RCF to about 250,000 RCF, about 150,000 RCF to about 200,000 RCF, about 150,000 RCF to about 250,000 RCF, or about 200,000 RCF to about 250,000 RCF. In some embodiments, the fixed angle centrifugation comprises centrifugation between about 10,000 RCF, about 20,000 RCF, about 30,000 RCF, about 40,000 RCF, about 50,000 RCF, about 60,000 RCF, about 70,000 RCF, about 80,000 RCF, about 100,000 RCF, about 150,000 RCF, about 200,000 RCF, or about 250,000 RCF. In some embodiments, the fixed angle centrifugation comprises centrifugation between at least about 10,000 RCF, about 20,000 RCF, about 30,000 RCF, about 40,000 RCF, about 50,000 RCF, about 60,000 RCF, about 70,000 RCF, about 80,000 RCF, about 100,000 RCF, about 150,000 RCF, or about 200,000 RCF. In some embodiments, the fixed angle centrifugation comprises centrifugation between at most about 20,000 RCF, about 30,000 RCF, about 40,000 RCF, about 50,000 RCF, about 60,000 RCF, about 70,000 RCF, about 80,000 RCF, about 100,000 RCF, about 150,000 RCF, about 200,000 RCF, or about 250,000 RCF. In some embodiments, the fixed angle centrifugation comprises about 124,000 RCF. In some embodiments, the fixed angle centrifugation comprises about 125,000 RCF. In some embodiments, the fixed angle centrifugation comprises about 126,000 RCF. In some embodiments, the fixed angle centrifugation comprises about 127,000 RCF. In some embodiments, the fixed angle centrifugation comprises about 128,000 RCF.

In some embodiments, the fixed angle centrifugation comprises an r average, an average RCF value at the middle of a tube during centrifugation, between about 10,000 RCF to about 150,000 RCF. In some embodiments, the fixed angle centrifugation comprises the r average between about 10,000 RCF to about 30,000 RCF, about 10,000 RCF to about 50,000 RCF, about 10,000 RCF to about 60,000 RCF, about 10,000 RCF to about 70,000 RCF, about 10,000 RCF to about 80,000 RCF, about 10,000 RCF to about 85,000 RCF, about 10,000 RCF to about 90,000 RCF, about 10,000 RCF to about 95,000 RCF, about 10,000 RCF to about 100,000 RCF, about 10,000 RCF to about 120,000 RCF, about 10,000 RCF to about 150,000 RCF, about 30,000 RCF to about 50,000 RCF, about 30,000 RCF to about 60,000 RCF, about 30,000 RCF to about 70,000 RCF, about 30,000 RCF to about 80,000 RCF, about 30,000 RCF to about 85,000 RCF, about 30,000 RCF to about 90,000 RCF, about 30,000 RCF to about 95,000 RCF, about 30,000 RCF to about 100,000 RCF, about 30,000 RCF to about 120,000 RCF, about 30,000 RCF to about 150,000 RCF, about 50,000 RCF to about 60,000 RCF, about 50,000 RCF to about 70,000 RCF, about 50,000 RCF to about 80,000 RCF, about 50,000 RCF to about 85,000 RCF, about 50,000 RCF to about 90,000 RCF, about 50,000 RCF to about 95,000 RCF, about 50,000 RCF to about 100,000 RCF, about 50,000 RCF to about 120,000 RCF, about 50,000 RCF to about 150,000 RCF, about 60,000 RCF to about 70,000 RCF, about 60,000 RCF to about 80,000 RCF, about 60,000 RCF to about 85,000 RCF, about 60,000 RCF to about 90,000 RCF, about 60,000 RCF to about 95,000 RCF, about 60,000 RCF to about 100,000 RCF, about 60,000 RCF to about 120,000 RCF, about 60,000 RCF to about 150,000 RCF, about 70,000 RCF to about 80,000 RCF, about 70,000 RCF to about 85,000 RCF, about 70,000 RCF to about 90,000 RCF, about 70,000 RCF to about 95,000 RCF, about 70,000 RCF to about 100,000 RCF, about 70,000 RCF to about 120,000 RCF, about 70,000 RCF to about 150,000 RCF, about 80,000 RCF to about 85,000 RCF, about 80,000 RCF to about 90,000 RCF, about 80,000 RCF to about 95,000 RCF, about 80,000 RCF to about 100,000 RCF, about 80,000 RCF to about 120,000 RCF, about 80,000 RCF to about 150,000 RCF, about 85,000 RCF to about 90,000 RCF, about 85,000 RCF to about 95,000 RCF, about 85,000 RCF to about 100,000 RCF, about 85,000 RCF to about 120,000 RCF, about 85,000 RCF to about 150,000 RCF, about 90,000 RCF to about 95,000 RCF, about 90,000 RCF to about 100,000 RCF, about 90,000 RCF to about 120,000 RCF, about 90,000 RCF to about 150,000 RCF, about 95,000 RCF to about 100,000 RCF, about 95,000 RCF to about 120,000 RCF, about 95,000 RCF to about 150,000 RCF, about 100,000 RCF to about 120,000 RCF, about 100,000 RCF to about 150,000 RCF, or about 120,000 RCF to about 150,000 RCF. In some embodiments, the fixed angle centrifugation comprises the r average between about 10,000 RCF, about 30,000 RCF, about 50,000 RCF, about 60,000 RCF, about 70,000 RCF, about 80,000 RCF, about 85,000 RCF, about 90,000 RCF, about 95,000 RCF, about 100,000 RCF, about 120,000 RCF, or about 150,000 RCF. In some embodiments, the fixed angle centrifugation comprises the r average between at least about 10,000 RCF, about 30,000 RCF, about 50,000 RCF, about 60,000 RCF, about 70,000 RCF, about 80,000 RCF, about 85,000 RCF, about 90,000 RCF, about 95,000 RCF, about 100,000 RCF, or about 120,000 RCF. In some embodiments, the fixed angle centrifugation comprises the r average between at most about 30,000 RCF, about 50,000 RCF, about 60,000 RCF, about 70,000 RCF, about 80,000 RCF, about 85,000 RCF, about 90,000 RCF, about 95,000 RCF, about 100,000 RCF, about 120,000 RCF, or about 150,000 RCF. In some embodiments, the fixed angle centrifugation comprises the r average of about 70,000 RCF. In some embodiments, the fixed angle centrifugation comprises the r average of about 75,000 RCF. In some embodiments, the fixed angle centrifugation comprises the r average of about 80,000 RCF. In some embodiments, the fixed angle centrifugation comprises the r average of about 85,000 RCF. In some embodiments, the fixed angle centrifugation comprises the r average of about 90,000 RCF. In some embodiments, the fixed angle centrifugation comprises the r average of about 95,000 RCF. In some embodiments, the fixed angle centrifugation comprises the r average of about 100,000 RCF.

In some embodiments, the fixed angle centrifugation comprises r max, an average RCF value at the bottom of a tube during centrifugation, between about 10,000 RCF to about 200,000 RCF. In some embodiments, the fixed angle centrifugation comprises the r max between about 10,000 RCF to about 30,000 RCF, about 10,000 RCF to about 50,000 RCF, about 10,000 RCF to about 60,000 RCF, about 10,000 RCF to about 80,000 RCF, about 10,000 RCF to about 100,000 RCF, about 10,000 RCF to about 120,000 RCF, about 10,000 RCF to about 130,000 RCF, about 10,000 RCF to about 140,000 RCF, about 10,000 RCF to about 160,000 RCF, about 10,000 RCF to about 180,000 RCF, about 10,000 RCF to about 200,000 RCF, about 30,000 RCF to about 50,000 RCF, about 30,000 RCF to about 60,000 RCF, about 30,000 RCF to about 80,000 RCF, about 30,000 RCF to about 100,000 RCF, about 30,000 RCF to about 120,000 RCF, about 30,000 RCF to about 130,000 RCF, about 30,000 RCF to about 140,000 RCF, about 30,000 RCF to about 160,000 RCF, about 30,000 RCF to about 180,000 RCF, about 30,000 RCF to about 200,000 RCF, about 50,000 RCF to about 60,000 RCF, about 50,000 RCF to about 80,000 RCF, about 50,000 RCF to about 100,000 RCF, about 50,000 RCF to about 120,000 RCF, about 50,000 RCF to about 130,000 RCF, about 50,000 RCF to about 140,000 RCF, about 50,000 RCF to about 160,000 RCF, about 50,000 RCF to about 180,000 RCF, about 50,000 RCF to about 200,000 RCF, about 60,000 RCF to about 80,000 RCF, about 60,000 RCF to about 100,000 RCF, about 60,000 RCF to about 120,000 RCF, about 60,000 RCF to about 130,000 RCF, about 60,000 RCF to about 140,000 RCF, about 60,000 RCF to about 160,000 RCF, about 60,000 RCF to about 180,000 RCF, about 60,000 RCF to about 200,000 RCF, about 80,000 RCF to about 100,000 RCF, about 80,000 RCF to about 120,000 RCF, about 80,000 RCF to about 130,000 RCF, about 80,000 RCF to about 140,000 RCF, about 80,000 RCF to about 160,000 RCF, about 80,000 RCF to about 180,000 RCF, about 80,000 RCF to about 200,000 RCF, about 100,000 RCF to about 120,000 RCF, about 100,000 RCF to about 130,000 RCF, about 100,000 RCF to about 140,000 RCF, about 100,000 RCF to about 160,000 RCF, about 100,000 RCF to about 180,000 RCF, about 100,000 RCF to about 200,000 RCF, about 120,000 RCF to about 130,000 RCF, about 120,000 RCF to about 140,000 RCF, about 120,000 RCF to about 160,000 RCF, about 120,000 RCF to about 180,000 RCF, about 120,000 RCF to about 200,000 RCF, about 130,000 RCF to about 140,000 RCF, about 130,000 RCF to about 160,000 RCF, about 130,000 RCF to about 180,000 RCF, about 130,000 RCF to about 200,000 RCF, about 140,000 RCF to about 160,000 RCF, about 140,000 RCF to about 180,000 RCF, about 140,000 RCF to about 200,000 RCF, about 160,000 RCF to about 180,000 RCF, about 160,000 RCF to about 200,000 RCF, or about 180,000 RCF to about 200,000 RCF. In some embodiments, the fixed angle centrifugation comprises the r max between about 10,000 RCF, about 30,000 RCF, about 50,000 RCF, about 60,000 RCF, about 80,000 RCF, about 100,000 RCF, about 120,000 RCF, about 130,000 RCF, about 140,000 RCF, about 160,000 RCF, about 180,000 RCF, or about 200,000 RCF. In some embodiments, the fixed angle centrifugation comprises the r max between at least about 10,000 RCF, about 30,000 RCF, about 50,000 RCF, about 60,000 RCF, about 80,000 RCF, about 100,000 RCF, about 120,000 RCF, about 130,000 RCF, about 140,000 RCF, about 160,000 RCF, or about 180,000 RCF. In some embodiments, the fixed angle centrifugation comprises the r max between at most about 30,000 RCF, about 50,000 RCF, about 60,000 RCF, about 80,000 RCF, about 100,000 RCF, about 120,000 RCF, about 130,000 RCF, about 140,000 RCF, about 160,000 RCF, about 180,000 RCF, or about 200,000 RCF. In some embodiments, the fixed angle centrifugation comprises the r max of about 100,000 RCF. In some embodiments, the fixed angle centrifugation comprises the r max of about 105,000 RCF. In some embodiments, the fixed angle centrifugation comprises the r max of about 110,000 RCF. In some embodiments, the fixed angle centrifugation comprises the r max of about 115,000 RCF. In some embodiments, the fixed angle centrifugation comprises the r max of about 120,000 RCF. In some embodiments, the fixed angle centrifugation comprises the r max of about 121,000 RCF. In some embodiments, the fixed angle centrifugation comprises the r max of about 122,000 RCF. In some embodiments, the fixed angle centrifugation comprises the r max of about 123,000 RCF. In some embodiments, the fixed angle centrifugation comprises the r max of about 124,000 RCF. In some embodiments, the fixed angle centrifugation comprises the r max of about 125,000 RCF. In some embodiments, the fixed angle centrifugation comprises the r max of about 126,000 RCF. In some embodiments, the fixed angle centrifugation comprises the r max of about 127,000 RCF. In some embodiments, the fixed angle centrifugation comprises the r max of about 128,000 RCF. In some embodiments, the fixed angle centrifugation comprises the r max of about 129,000 RCF. In some embodiments, the fixed angle centrifugation comprises the r max of about 130,000 RCF. In some embodiments, the fixed angle centrifugation comprises the r max of about 135,000 RCF. In some embodiments, the fixed angle centrifugation comprises the r max of about 140,000 RCF. In some embodiments, the fixed angle centrifugation comprises the r max of about 145,000 RCF. In some embodiments, the fixed angle centrifugation comprises the r max of about 150,000 RCF.

In some embodiments, the methods for cell processing further include generating the density gradient by centrifuging a density gradient media with acceleration spanning over at least about 1 minute (min), at least about 5 min, at least about 9 min, at least about 10 min, at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min. In some embodiments, the methods for cell processing further include generating the density gradient by centrifuging a polysaccharide with acceleration spanning over at least about 1 min, at least about 5 min, at least about 9 min, at least about 10 min, at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min. In some embodiments, the methods for cell processing further include generating the density gradient by centrifuging a polysaccharide with acceleration spanning over at least about 9 min, at least about 10 min, at least about 20 min, at least about 30 min, at least about 40 min, or at least about 50 min.

In some embodiments, the methods for cell processing further include generating the density gradient by centrifuging a density gradient media with deceleration spanning over at least about 1 minute (min), at least about 5 min, at least about 9.5 min, at least about 10 min, at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min. In some embodiments, the methods for cell processing further include generating the density gradient by centrifuging a polysaccharide with deceleration spanning over at least about 1 min, at least about 5 min, at least about 9.5 min, at least about 10 min, at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min. In some embodiments, the methods for cell processing further include generating the density gradient by centrifuging a polysaccharide with deceleration spanning over at least about 9.5 min, at least about 10 min, at least about 20 min, at least about 30 min, at least about 40 min, or at least about 50 min.

In some embodiments, the fixed angle centrifugation comprises centrifugation for about 10 minutes to about 120 minutes. In some embodiments, the fixed angle centrifugation comprises centrifugation for about 20 minutes to about 110 minutes. In some embodiments, the fixed angle centrifugation comprises centrifugation for about 30 minutes to about 100 minutes. In some embodiments, the fixed angle centrifugation comprises centrifugation for about 40 minutes to about 90 minutes. In some embodiments, the fixed angle centrifugation comprises centrifugation for about 50 minutes to about 80 minutes. In some embodiments, the fixed angle centrifugation comprises centrifugation for about 50 minutes to about 70 minutes. In some embodiments, the fixed angle centrifugation comprises centrifugation for about 50 minutes to about 60 minutes.

In some embodiments, the fixed angle centrifugation comprises centrifugation for about 10 minutes. In some embodiments, the fixed angle centrifugation comprises centrifugation for about 20 minutes. In some embodiments, the fixed angle centrifugation comprises centrifugation for about 30 minutes. In some embodiments, the fixed angle centrifugation comprises centrifugation for about 40 minutes. In some embodiments, the fixed angle centrifugation comprises centrifugation for about 50 minutes. In some embodiments, the fixed angle centrifugation comprises centrifugation for about 60 minutes. In some embodiments, the fixed angle centrifugation comprises centrifugation for about 70 minutes. In some embodiments, the fixed angle centrifugation comprises centrifugation for about 80 minutes. In some embodiments, the fixed angle centrifugation comprises centrifugation for about 90 minutes. In some embodiments, the fixed angle centrifugation comprises centrifugation for about 100 minutes. In some embodiments, the fixed angle centrifugation comprises centrifugation for about 110 minutes. In some embodiments, the fixed angle centrifugation comprises centrifugation for about 120 minutes.

In some embodiments, the fixed angle centrifugation comprises an acceleration, comprising from about 1.0 min to about 10.0 min to reach the RCF for centrifugation. In some embodiments, the fixed angle centrifugation comprises acceleration, comprising about 1.0 min to reach RCF for centrifugation. In some embodiments, the fixed angle centrifugation comprises acceleration, comprising about 1.5 min to reach RCF for centrifugation. In some embodiments, the fixed angle centrifugation comprises acceleration, comprising about 2.0 min to reach RCF for centrifugation. In some embodiments, the fixed angle centrifugation comprises acceleration, comprising about 2.5 min to reach RCF for centrifugation. In some embodiments, the fixed angle centrifugation comprises acceleration, comprising about 3.0 min to reach RCF for centrifugation. In some embodiments, the fixed angle centrifugation comprises acceleration, comprising about 3.5 min to reach RCF for centrifugation. In some embodiments, the fixed angle centrifugation comprises acceleration, comprising about 4.0 min to reach RCF for centrifugation. In some embodiments, the fixed angle centrifugation comprises acceleration, comprising about 4.5 min to reach RCF for centrifugation. In some embodiments, the fixed angle centrifugation comprises acceleration, comprising about 5.0 min to reach RCF for centrifugation. In some embodiments, the fixed angle centrifugation comprises acceleration, comprising about 5.2 min to reach RCF for centrifugation. In some embodiments, the fixed angle centrifugation comprises acceleration, comprising about 5.5 min to reach RCF for centrifugation. In some embodiments, the fixed angle centrifugation comprises acceleration, comprising about 6.0 min to reach RCF for centrifugation. In some embodiments, the fixed angle centrifugation comprises acceleration, comprising about 6.5 min to reach RCF for centrifugation. In some embodiments, the fixed angle centrifugation comprises acceleration, comprising about 7.0 min to reach RCF for centrifugation. In some embodiments, the fixed angle centrifugation comprises acceleration, comprising about 7.5 min to reach RCF for centrifugation. In some embodiments, the fixed angle centrifugation comprises acceleration, comprising about 8.0 min to reach RCF for centrifugation. In some embodiments, the fixed angle centrifugation comprises acceleration, comprising about 8.5 min to reach RCF for centrifugation. In some embodiments, the fixed angle centrifugation comprises acceleration, comprising about 9.0 min to reach RCF for centrifugation. In some embodiments, the fixed angle centrifugation comprises acceleration, comprising about 9.5 min to reach RCF for centrifugation. In some embodiments, the fixed angle centrifugation comprises acceleration, comprising about 10.0 min to reach RCF for centrifugation.

In some embodiments, the fixed angle centrifugation comprises deceleration comprising from about 1.0 min to about 10.0 min to reach a stoppage of the fixed angle centrifugation. In some embodiments, the fixed angle centrifugation comprises deceleration comprising about 1.0 min to reach a stoppage of the fixed angle centrifugation. In some embodiments, the fixed angle centrifugation comprises deceleration comprising about 1.5 min to reach a stoppage of the fixed angle centrifugation. In some embodiments, the fixed angle centrifugation comprises deceleration comprising about 2.0 min to reach a stoppage of the fixed angle centrifugation. In some embodiments, the fixed angle centrifugation comprises deceleration comprising about 2.5 min to reach a stoppage of the fixed angle centrifugation. In some embodiments, the fixed angle centrifugation comprises deceleration comprising about 3.0 min to reach a stoppage of the fixed angle centrifugation. In some embodiments, the fixed angle centrifugation comprises deceleration comprising about 3.5 min to reach a stoppage of the fixed angle centrifugation. In some embodiments, the fixed angle centrifugation comprises deceleration comprising about 4.0 min to reach a stoppage of the fixed angle centrifugation. In some embodiments, the fixed angle centrifugation comprises deceleration comprising about 4.5 min to reach a stoppage of the fixed angle centrifugation. In some embodiments, the fixed angle centrifugation comprises deceleration comprising about 5.0 min to reach a stoppage of the fixed angle centrifugation. In some embodiments, the fixed angle centrifugation comprises deceleration comprising about 5.5 min to reach a stoppage of the fixed angle centrifugation. In some embodiments, the fixed angle centrifugation comprises deceleration comprising about 6.0 min to reach a stoppage of the fixed angle centrifugation. In some embodiments, the fixed angle centrifugation comprises deceleration comprising about 6.5 min to reach a stoppage of the fixed angle centrifugation. In some embodiments, the fixed angle centrifugation comprises deceleration comprising about 7.0 min to reach a stoppage of the fixed angle centrifugation. In some embodiments, the fixed angle centrifugation comprises deceleration comprising about 7.5 min to reach a stoppage of the fixed angle centrifugation. In some embodiments, the fixed angle centrifugation comprises deceleration comprising about 8.0 min to reach a stoppage of the fixed angle centrifugation. In some embodiments, the fixed angle centrifugation comprises deceleration comprising about 8.5 min to reach a stoppage of the fixed angle centrifugation. In some embodiments, the fixed angle centrifugation comprises deceleration comprising about 9.0 min to reach a stoppage of the fixed angle centrifugation. In some embodiments, the fixed angle centrifugation comprises deceleration comprising about 9.5 min to reach a stoppage of the fixed angle centrifugation. In some embodiments, the fixed angle centrifugation comprises deceleration comprising about 10.0 min to reach a stoppage of the fixed angle centrifugation.

In some embodiments, the methods for cell processing disclosed herein include enucleating a portion of the nucleated cells to produce an enucleated cell fraction using fixed angle centrifugation, including continuous flow centrifugation, wherein the portion of the nucleated cells that is enucleated is greater than or equal to about 10% of the nucleated cells. In some embodiment, the portion of the nucleated cells is greater than or equal to about 20% of the nucleated cells. In some embodiment, the portion of the nucleated cells is greater than or equal to about 25% of the nucleated cells. In some embodiment, the portion of the nucleated cells is greater than or equal to about 30% of the nucleated cells. In some embodiment, the portion of the nucleated cells is greater than or equal to about 35% of the nucleated cells. In some embodiment, the portion of the nucleated cells is greater than or equal to about 40% of the nucleated cells. In some embodiment, the portion of the nucleated cells is greater than or equal to about 45% of the nucleated cells. In some embodiment, the portion of the nucleated cells is greater than or equal to about 50% of the nucleated cells. In some embodiment, the portion of the nucleated cells is greater than or equal to about 55% of the nucleated cells. In some embodiment, the portion of the nucleated cells is greater than or equal to about 60% of the nucleated cells. In some embodiment, the portion of the nucleated cells is greater than or equal to about 65% of the nucleated cells. In some embodiment, the portion of the nucleated cells is greater than or equal to about 70% of the nucleated cells. In some embodiment, the portion of the nucleated cells is greater than or equal to about 75% of the nucleated cells. In some embodiment, the portion of the nucleated cells is greater than or equal to about 80% of the nucleated cells. In some embodiment, the portion of the nucleated cells is greater than or equal to about 85% of the nucleated cells. In some embodiment, the portion of the nucleated cells is greater than or equal to about 90% of the nucleated cells. In some embodiment, the portion of the nucleated cells is greater than or equal to about 95% of the nucleated cells.

In some embodiments, the enucleated cell fraction produced by methods disclosed herein comprises more than or equal to about 1×10⁵ enucleated cells, 1×10⁶ enucleated cells, about 1×10⁷ enucleated cells, 3×10⁵ enucleated cells, 5×10⁵ enucleated cells, 7×10⁷ of enucleated cells, 8×10⁷ of enucleated cells, 9×10⁷ of enucleated cells, 10×10⁷ of enucleated cells, 15×10⁷ of enucleated cells, 20×10⁷ of enucleated cells, 50×10⁷ of enucleated cells, 70×10⁷ enucleated cells, 90×10⁷ enucleated cells, 100×10⁷ of enucleated cells, 150×10⁷ of enucleated cells, 200×10⁷ of enucleated cells, 250×10⁷ enucleated cells, 300×10⁷ enucleated cells, or 500×10⁷ of enucleated cells.

In some embodiments, the enucleated cell of the enucleated cell fraction has a diameter comprising less than or equal to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of an average diameter of the nucleated cells. In some embodiments, the enucleated cell of the enucleated cell fraction has a diameter comprising less than or equal to about 10%, about 20%, about 30%, about 40%, about 50%, about 60%, about 70%, about 80%, about 90%, about 95%, or about 99% of an average diameter of the nucleated cells. In some embodiments, the enucleated cell of the enucleated cell fraction has a diameter comprising less than or equal to about 50% of an average diameter of the nucleated cells. In some embodiments, the enucleated cell of the enucleated cell fraction has a diameter comprising less than or equal to about 60% of an average diameter of the nucleated cells. In some embodiments, the enucleated cell of the enucleated cell fraction has a diameter comprising less than or equal to about 70% of an average diameter of the nucleated cells. In some embodiments, the enucleated cell of the enucleated cell fraction has a diameter comprising less than or equal to about 80% of an average diameter of the nucleated cells. In some embodiments, the enucleated cell of the enucleated cell fraction has a diameter comprising less than or equal to about 90% of an average diameter of the nucleated cells.

In some embodiments, the enucleated cell of the enucleated cell fraction has a diameter comprising more than or equal to about 5 μm, about 10 μm, about 20 μm, about 30 μm, about 40 μm, about 50 μm, about 60 μm, about 70 μm, about 80 μm, or about 90 μm. In some embodiments, the enucleated cell of the enucleated cell fraction has a diameter ranging from about 1 μm to about 10 μm. In some embodiments, the enucleated cell of the enucleated cell fraction has a diameter ranging from about 1 μm to about 2 μm, about 1 μm to about 3 μm, about 1 μm to about 4 μm, about 1 μm to about 5 μm, about 1 μm to about 6 μm, about 1 μm to about 7 μm, about 1 μm to about 8 μm, about 1 μm to about 9 μm, about 1 μm to about 10 μm, about 2 μm to about 3 μm, about 2 μm to about 4 μm, about 2 μm to about 5 μm, about 2 μm to about 6 μm, about 2 μm to about 7 μm, about 2 μm to about 8 μm, about 2 μm to about 9 μm, about 2 μm to about 10 μm, about 3 μm to about 4 μm, about 3 μm to about 5 μm, about 3 μm to about 6 μm, about 3 μm to about 7 μm, about 3 μm to about 8 μm, about 3 μm to about 9 μm, about 3 μm to about 10 μm, about 4 μm to about 5 μm, about 4 μm to about 6 μm, about 4 μm to about 7 μm, about 4 μm to about 8 μm, about 4 μm to about 9 μm, about 4 μm to about 10 μm, about 5 μm to about 6 μm, about 5 μm to about 7 μm, about 5 μm to about 8 μm, about 5 μm to about 9 μm, about 5 μm to about 10 μm, about 6 μm to about 7 μm, about 6 μm to about 8 μm, about 6 μm to about 9 μm, about 6 μm to about 10 μm, about 7 μm to about 8 μm, about 7 μm to about 9 μm, about 7 μm to about 10 μm, about 8 μm to about 9 μm, about 8 μm to about 10 μm, or about 9 μm to about 10 μm. In some embodiments, the enucleated cell of the enucleated cell fraction has a diameter ranging from about 1 μm, about 2 μm, about 3 μm, about 4 μm, about 5 μm, about 6 μm, about 7 μm, about 8 μm, about 9 μm, or about 10 μm. In some embodiments, the enucleated cell of the enucleated cell fraction has a diameter ranging from at least about 1 μm, about 2 μm, about 3 μm, about 4 μm, about 5 μm, about 6 μm, about 7 μm, about 8 μm, or about 9 μm. In some embodiments, the enucleated cell of the enucleated cell fraction has a diameter ranging from at most about 2 μm, about 3 μm, about 4 μm, about 5 μm, about 6 μm, about 7 μm, about 8 μm, about 9 μm, or about 10 μm. In some embodiments, the enucleated cell of the enucleated cell fraction has a diameter of about 8 μm.

### Modifying Cells of the Disclosure

As shown in FIG. 1, a nucleated (“parent”) cell may be engineered prior to enucleation to express one or more exogenous agents, or after enucleation, or a combination thereof. In some embodiments, the one or more exogenous biomolecule comprises a targeting moiety, a transmembrane moiety, or a therapeutic agent, or a combination thereof. In some embodiments, the targeting moiety comprises an adhesion molecule, chemokine or retention receptors or both. In some embodiments, the targeting moiety is engineered to target a target tissue, cell or environment disclosed herein (e.g., the lymph tissue in a subject). In addition, or alternatively, the resulting enucleated cell is engineered to express and, in some cases, to secrete the therapeutic agent. In some embodiments, the therapeutic agent comprises an antibody or an antigen-binding fragment thereof (e.g., single-domain antibody). In some embodiments, the enucleated cell may be administered to a subject in need thereof to treat a disease or a condition in the subject.

Various methods may be used to introduce a biomolecule (e.g., the therapeutic agent, transmembrane moiety, immune-evading moiety, and/or targeting moiety described herein) into the parent cell or the enucleated cell described herein. Non-limiting examples of methods that may be used to introduce a biomolecule into the parent cell or the enucleated cell include: liposome mediated transfer, an adenovirus, an adeno-associated virus, a herpes virus, a retroviral based vector, a lentiviral vector, electroporation, microinjection, lipofection, transfection, calcium phosphate transfection, dendrimer-based transfection, cationic polymer transfection, cell squeezing, sonoporation, optical transfection, impalection, hydrodynamic delivery, magnetofection, nanoparticle transfection, or combinations thereof. In some aspects of any of the compositions and methods provided herein, a therapeutic agent, a virus, an antibody, or a nanoparticle may be introduced into the enucleated cells.

In some embodiments, the enucleated cell is preserved via cryopreservation, cryohibernation, or lyophilization. Cryopreservation comprises freezing the enucleated cell, while cryohibernation comprises storing the enucleated cell at a temperature that is below room temperature but without freezing the enucleated cell. In some embodiments, the enucleated cell is lyophilized. In some embodiments, the lyophilized enucleated cell can be reconstituted, and the reconstituted enucleated cell exhibits comparable viability to the enucleated cell that has not been lyophilized. In some embodiments, the lyophilization comprises components: freezing the cell; subjecting the cell to drying under a very low pressure (e.g., <3000 mTorr) using vacuum. The drying component can lead to sublimation and dehydrate the cell while maintaining cellular viability and biologic function. In some embodiments, the freezing phase comprises balancing the duration and temperature of the freezing to for maintaining cell viability and stability, appropriate crystal formation, and the speed of reconstitution. The triple point of a substance is the temperature and the pressure at which the sublimation curve, fusion curve, and vaporization curve meet. Achievement of the triple point which varies for different substances ensures that sublimation rather than melting will occur in the following drying steps. To facilitate faster and more efficient freeze-drying, larger ice crystals are preferred, because they form a network within the product that promotes faster removal of water vapor during sublimation. To produce larger crystals, the product should be frozen slowly or the temperature can be cycled up and down in a process called annealing. Fresh or frozen living tissue or cells do not have a single homogeneous melting point (eutectic point) and consequently the freezing stage of the material (cells or tissue) is cooled below its triple point which represents the temperature and pressure at which the solid, liquid and gas phases of the material can coexist. Living cells do have a critical point on a phase diagram at which both the liquid and the gas phase of an object or substance have the same density and are therefore indistinguishable. The product critical point temperature must be maintained to prevent melt-back or cake collapse occurring during primary and secondary drying which reflects incomplete sublimation. In the case of substances where preservation of structure is required like living cells, large ice crystals maybe detrimental and may break the cell walls which can result in increasingly poor texture and loss of nutritive content. In this case, the freezing should be done rapidly, in order to lower the material to below its critical point quickly, thus avoiding the formation of large ice crystals. The freezing temperatures for cells or tissue can vary but ranges in general between −50° C. (−58° F.) and −80° C. (−112° F.).

During the drying phase, the ambient pressure is lowered to the range of a few millibars, and then heat is supplied by conduction or radiation to the material for the ice to sublime. The amount of heat necessary can be calculated using the sublimating molecules' latent heat of sublimation. In this initial drying phase, about 95% of the water in the material or substance is sublimated. This phase is often slow and can even last for several days depending on the substance and technology employed but if too much heat is added to quickly the material's structure could be altered. In this phase, pressure is controlled through the application of a partial vacuum. The vacuum speeds up the sublimation, making it useful as a deliberate drying process. A cold condenser chamber and/or condenser plates are used as a surface(s) for the water vapor to re-liquify and solidify on. It is important to note that in this range of pressure, the heat cannot be provided by a convection effect because of the low air density. The drying phase also aims to remove remaining unfrozen water molecules since the ice induced with freezing should be removed during the primary drying phase. This part of the freeze-drying process is governed by the material's adsorption isotherms. In this phase, the temperature is raised higher than in the primary drying phase and can even be above 0° C. (32° F.), to break any physico-chemical interactions that have formed between the water molecules and the frozen material. Usually, during this phase the pressure is also lowered in this stage to encourage desorption. However, there are products that benefit from increased pressure as well. After the freeze-drying process is complete, the vacuum is usually broken with an inert gas, such as nitrogen, before the material is sealed. At the end of the operation, the residual water content in the product is extremely low and should range from <1% to 4% of the original concentration.

In some embodiments, the lyophilization of the enucleated cell comprises the use of lyoprotectants for retaining cell viability and biologic function. Lyoprotectant comprises addition of reagents, salts, or additives that protects cell during the desiccation process. Common lyoprotectants include trehalose, DMSO, methylcellulose, sucrose, antioxidants, human or animal serum proteins, and cellular stress proteins. Additionally, methods for increasing the transport of lyoprotectants inside the cells in suspension can be utilized as a way of improving the viability and function of cells after lyophilization. These methods include electroporation, addition of reagents that enhance intracellular transport, genetic modification of cells to upregulate the expression of pores on cell membranes, and mechanical microfluidic devices that partially disrupt cell membrane integrity and potentially promote intracellular transport of lyoprotectants.

In some embodiments, the nucleated cell described herein can be modified to express a targeting moiety (e.g., an antibody or antigen binding fragment thereof), a therapeutic agent, a transmembrane moiety, a heterologous gene product, or a combination thereof. In some embodiments, the nucleated cell can be modified to express at least one heterologous polynucleotide, where the at least one heterologous polynucleotide encodes a targeting moiety, a therapeutic agent, a transmembrane moiety, heterologous gene product, or a combination thereof.

In one aspect, disclosed herein are methods for modifying a cell by introducing at least one heterologous polynucleotide into the cell. In some embodiments, the heterologous polynucleotide encodes promoter, a heterologous gene product, or a combination thereof. In some embodiments, the methods comprise providing a composition comprising a first subset of nucleated cells and enucleated cells derived from a second subset of the nucleated cells. In some embodiments, the first subset of the nucleated cells comprises a heterologous polynucleotide encoding a heterologous gene product. The heterologous polynucleotide can be introduced into any types of cells that could be enucleated, for instance, but not limited to, hTERT-immobilized Mesenchymal stem cells, by transfection of plasmids, transposons, zinc finger nucleases (ZFNs), transcription activator-like effector nucleases (TALENs), CRISPR-Cas technologies, viral transduction, etc.

In some embodiments, the heterologous polynucleotide comprises a promoter. In some embodiments, the promoter comprises an inducible promoter. In some embodiments, the promoter should be compatible with mammalian gene expression, provide rapid, strong gene expression, only in the presence of its induction stimulus. In some embodiments, the inducible promoter is hypothermic. In some embodiments, the inducible promoter is induced by contacting the nucleated cells with a temperature that is below about 40° C. In some embodiments, the inducible promoter is induced by contacting the nucleated cells with a temperature that is below about 39° C. In some embodiments, the inducible promoter is induced by contacting the nucleated cells with a temperature that is below about 38° C. In some embodiments, the inducible promoter is induced by contacting the nucleated cells with a temperature that is below about 37° C. In some embodiments, the inducible promoter is induced by contacting the nucleated cells with a temperature that is below about 36° C. In some embodiments, the inducible promoter is induced by contacting the nucleated cells with a temperature that is below about 35° C. In some embodiments, examples of the inducible promoter include, but not limited to, comprises dsrA or CIRP.

In some embodiments, the inducible promoter is hyperthermic. In some embodiments, the inducible promoter is induced by contacting the nucleated cells with a temperature that is above 35° C. In some embodiments, the inducible promoter is induced by contacting the nucleated cells with a temperature that is above 36° C. In some embodiments, the inducible promoter is induced by contacting the nucleated cells with a temperature that is above 37° C. In some embodiments, the inducible promoter is induced by contacting the nucleated cells with a temperature that is above 38° C. In some embodiments, the inducible promoter is induced by contacting the nucleated cells with a temperature that is above 39° C. In some embodiments, the inducible promoter is induced by contacting the nucleated cells with a temperature that is above 40° C. In some embodiments, examples of the inducible promoter include, but not limited to, heat shock protein 70 (HSP70), heat shock protein 90 (HSP90), growth arrest- and DNA damage-inducible gene 153 (GADD153), multidrug resistance mutation 1 (MDR1), or cytomegalovirus (HSE-CMV).

In some embodiments, the inducible promoter is induced by contacting the nucleated cells with a molecule. In some embodiments, examples of the molecule include, but not limited to, rtTA, TRE, TetR, Cumate, Rapamycin, Abscisic acid, IPTG, or Methallothionein.

In some embodiments, the inducible promoter is induced by contacting the nucleated cells with light. In some embodiments, examples of the inducible promoter include, but not limited to, CIB1-CRY2 or GAL4-VVD.

In some embodiments, the inducible promoter is induced by contacting with the nucleated cells with a hormone. In some embodiments, examples of the inducible promoter include, but not limited to, Estradiol-Gal4.

In some embodiments, the promoter comprises a constitutively active promoter. The promoter will be continually active, but suicide will be induced under certain circumstances. In some embodiments, the constitutively active promoter is configured to activate transcription of the heterologous polynucleotide under conditions sufficient to express the heterologous gene product. In some embodiments, the enucleated cell described herein can be cryopreserved, cryohibernated, lyophilized, or a combination thereof. In some embodiments, the cryopreserved enucleated cell, following thawing, the enucleated cell is as viable as an otherwise comparable enucleated cell that is not cryopreserved. In some embodiments, the lyophilized enucleated cell is as viable as an otherwise comparable enucleated cell that is not lyophilized. In some embodiments, the cryohibernated enucleated cell is as viable as an otherwise comparable enucleated cell that is not cryohibernated.

### Method of Treatment

Disclosed herein, in some embodiments, are methods of using the enucleated cell, the composition, or the pharmaceutical composition described herein for treating a disease or condition. In some embodiments, the methods include treating the disease or condition of the subject by administering a composition described herein (e.g., a pharmaceutical composition containing enucleated cells engineered to express a therapeutic agent) to the subject.

The present disclosure also provides methods for the use of enucleated cells (natural or enucleated) as fusion partners to other cells (therapeutic or natural) to enhance and/or transfer biomolecules described herein, such as for example, a therapeutic agent. In some embodiments, the biomolecules include, DNA/genes, RNA (mRNA, shRNA, siRNA, miRNA), nanoparticles, peptides, proteins, and plasmids, bacteria, viruses, small molecule drugs, ions, cytokines, growth factors, and hormones. In some embodiments, the enucleated cell is engineered to express a fusogenic moiety. The fusogenic moiety can be any biomolecule (e.g., sugar, lipid, or protein) that promotes fusion of the membrane. In some embodiments, the fusogenic moiety is a fusogenic protein. A fusogenic protein allows the enucleated cell expressing the fusogenic protein to fuse with a target cell. In some embodiments, the fusogenic protein facilitates the merging of an enucleated cell expressing the fusogenic protein with a target cell, allowing the contents of the enucleated cell to enter into the target cell. In some embodiments, the fusogenic protein is heterotypic such as viral classes I-III or HAP2/GCS1 or SNARE. In some embodiments, the fusogenic protein is homoleptic such as EFF-1/AFF-1. Other non-limiting examples of the fusogenic protein is Izumo1 or Syncytin. In some embodiments, the fusogenic protein is a viral protein. In some embodiments, the fusogenic protein from a virus is VSV-g, hERV-W-ENV (Syncytin), or MV-Ed-F+MV-Ed-H (Hemagglutinin). Unlike nucleated cells, the fusion of enucleated cells to the same or another cell type of similar or different origin generates a unique cell hybrid that lacks problematic nuclear transfer, while maintaining desirable therapeutic attributes including, but not limited to, cell surface proteins, signal transduction molecules, secreted proteins, and epigenetic changes.

### Subject

In practicing the methods of treatment or use provided herein, therapeutically effective amounts of pharmaceutical compositions described herein are administered to a mammal having a disease, disorder, or condition to be treated, e.g., cancer. In some embodiments, the mammal is a human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

### Disease or Condition

Provided herein are methods of treating a disease or a condition in a subject by administering a composition described herein (e.g., a pharmaceutical composition containing enucleated cells engineered to express a therapeutic agent) to the subject. Provided herein are methods of treating cancer in a subject by administering a composition described herein (e.g., a pharmaceutical composition containing enucleated cells engineered to express a therapeutic agent) to the subject. Provided herein are methods of treating a lung disease in a subject by administering a composition described herein (e.g., a pharmaceutical composition containing enucleated cells engineered to express a therapeutic agent) to the subject. In some embodiments, administration is by any suitable mode of administration, including systemic administration (e.g., intravenous, inhalation, etc.). In some embodiments, the subject is human.

In some embodiments, the disease or the condition comprises an infection (e.g., human immunodeficiency virus (HIV)-infection, Chagas disease, tuberculosis), a neurological disease (e.g., Parkinson's Disease, Huntington's Disease, Alzheimer's Disease) an autoimmune disease (e.g., diabetes, Crohn's disease, multiple sclerosis, sickle cell anemia), a cardiovascular disease (e.g., acute myocardial infarction, heart failure, refractory angina), a ophthalmologic disease, a skeletal disease, a metabolic disease (e.g., phenylketonuria, glycogen storage deficiency type 1A, Gaucher disease), an inflammatory disease (e.g., cancer, inflammatory bowel disease), or a disease caused by external pathogen or toxin in a subject. In some embodiments, the disease or the condition comprises idiopathic pulmonary fibrosis. In some embodiments, the subject is in need of, or has been determined to be in need of, such an enucleated cell treatment.

In some embodiments, the cancer may be lung cancer, including non-small cell lung cancer (NSCLC), small cell lung cancer (SCLC), or any other lung cancer type. For example, the lung cancer may include adenocarcinoma, squamous carcinoma, large cell (undifferentiated) carcinoma, large cell neuroendocrine carcinoma, adenosquamous carcinoma, sarcomatoid carcinoma, lung carcinoid tumor, or adenoid cystic carcinoma. Other non-limiting example of lung cancer includes lymphoma, sarcoma, benign lung tumor, or hamartoma. In some embodiments, the cancer is metastatic cancer. In some embodiments, the cancer metastasized to the lung from a different tissue or source. For example, the metastatic cancer that may be found in the lung may include breast cancer, colon cancer, prostate cancer, sarcoma, bladder cancer, neuroblastoma, and Wilm's tumor.

In some embodiments, the enucleated cell described herein comprises a targeting moiety described herein for binding to an epitope expressed by a cancer cell or an epitope associated with a tumor microenvironment. In some embodiments, the targeting moiety comprises an antibody or antigen-binding fragment thereof described herein. In some embodiments, the antibody or antigen-binding fragment thereof comprises a single-domain antibody. In some embodiments, the antibody or antigen-binding fragment binds to an epitope expressed by a cancer cell or an epitope associated with a tumor microenvironment. In some embodiments, the binding of the targeting moiety (e.g., the antibody or the antigen-binding fragment thereof) to the epitope provides a therapeutic effect to treat cancer in a subject. In some embodiments, the binding of the targeting moiety (e.g., antibody or the antigen-binding fragment thereof) to the epitope recruits immune cells to activate immune response against the cancer.

In some embodiments, described herein are enucleated cells and methods of using these enucleated cells to treat a disease or condition associated with abnormal vasculature in a subject. Abnormal vasculature can be associated with disease or condition such as inflammation and cancer (e.g., any one of the cancers described herein). In some embodiments, the enucleated cells described herein, when contacted with the abnormal vasculature, increases the normalization of the abnormal vasculature, where the adhesion between endothelial cells is increased to prevent leakage of intravascular factors out of the vasculature. In some embodiments, the normalization of the abnormal vasculature includes decreasing of damages such as cell dead of the endothelial cells of the vasculature. In some embodiments, the normalization of the abnormal vasculature includes angiogenesis of immature or leaky vessels. In some embodiments, the normalization exerted by the enucleated cells can include normalization of blood vessel, lymphatic vessel, or a combination thereof.

In some embodiments, the disease or condition may be caused by a pathogen. In some embodiments, the enucleated cell described herein comprises an antibody or an antigen-binding fragment thereof or single-domain antibody that binds to an epitope expressed by the pathogen or an epitope associated with a microenvironment associated with the pathogen. In some cases, the binding of the antibody or the antigen-binding fragment thereof or single-domain antibody to the epitope confers therapeutic property against the pathogen. In some embodiments, the binding of the antibody or the antigen-binding fragment thereof or single-domain antibody to the epitope recruits immune cells to activate immune response to confer therapeutic property against the pathogen. For example, the disease or condition may be caused by virus, bacterium, fungus, parasite, or molecule resulted from detoxification. In some embodiments, the pathogens: may be disseminated or transmitted from person to person; result in high mortality rates and have the potential for major public health impact; may cause public panic and social disruption; and require special action for public health preparedness. Example of these pathogens may include Anthrax (Bacillus anthracis), Botulism (Clostridium botulinum toxin), Plague (Yersinia pestis), Smallpox (variola major), Tularemia (Francisella tularensis), or Viral hemorrhagic fevers, including Filoviruses (Ebola, Marburg) and Arenaviruses (Lassa, Machupo).

In some embodiments, the pathogen: may be disseminated; resulting in moderate morbidity rates and low mortality rates; and require specific enhancements of diagnostic capacity and enhanced disease surveillance. Example of these pathogens may include Brucellosis (Brucella species), Epsilon toxin of Clostridium perfringens, Food safety threats (e.g., Salmonella species, Escherichia coli 0157:H7, or Shigella), Glanders (Burkholderia mallei), Melioidosis (Burkholderia pseudomallei), Psittacosis (Chlamydia psittaci), Q fever (Coxiella burnetii), Ricin toxin from Ricinus communis (castor beans), Staphylococcal enterotoxin B, Typhus fever (Rickettsia prowazekii), Viral encephalitis (alphaviruses, such as eastern equine encephalitis, Venezuelan equine encephalitis, and western equine encephalitis), or Water safety threats (e.g., Vibrio cholerae and Cryptosporidium parvum).

In some embodiments, the pathogen may include emerging pathogen that has a high potential for mortality and morbidity, but the extend of which is not fully understood. Non-limiting examples of these pathogens may include Nipah virus and hantavirus.

The enucleated cells described herein, or the composition containing such enucleated cells (referred to in this section as “composition”) may be administered to a subject in a suitable dose, mod of administration, and frequency, which depends on the intended effect.

In some embodiments, the composition is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, or 100 times) during a period of time.

In some embodiments, the composition is administered in a therapeutically-effective amount by various forms and routes including, for example, oral, or topical administration. In some embodiments, a composition may be administered by parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the composition is delivered via multiple administration routes.

In some embodiments, the composition is administered by intravenous infusion. In some embodiments, the composition is administered by slow continuous infusion over a long period, such as more than 24 hours. In some embodiments, the composition is administered as an intravenous injection or a short infusion.

A composition may be administered in a local manner, for example, via injection of the agent directly into an organ, optionally in a depot or sustained release formulation or implant. A composition may be provided in the form of a rapid release formulation, in the form of an extended release formulation, or in the form of an intermediate release formulation. A rapid release form may provide an immediate release. An extended release formulation may provide a controlled release or a sustained delayed release. In some embodiments, a pump may be used for delivery of the composition. In some embodiments, a pen delivery device may be used, for example, for subcutaneous delivery of a composition of the disclosure. The composition provided herein may be administered in conjunction with other therapies, for example, an antiviral therapy, a chemotherapy, an antibiotic, a cell therapy, a cytokine therapy, or an anti-inflammatory agent.

The compositions (e.g., enucleated cells or pharmaceutical composition comprising the enucleated cell described herein) may be administered before, during, or after the occurrence of a disease or condition, and the timing of administering the composition containing a therapeutic agent may vary. In some cases, the composition may be used as a prophylactic and may be administered continuously to subjects (e.g., the subject for immunization or the subject for treatment) with a susceptibility to a coronavirus or a propensity to a condition or disease associated with a coronavirus. Prophylactic administration may lessen a likelihood of the occurrence of the infection, disease or condition, or may reduce the severity of the infection, disease or condition.

The composition may be administered to a subject before the onset of the symptoms. The composition may be administered to a subject (e.g., the subject for immunization or the subject for treatment) after (e.g., as soon as possible after) a test result, for example, a test result that provides a diagnosis, a test that shows the presence of a coronavirus in a subject (e.g., the subject for immunization or the subject for treatment), or a test showing progress of a condition, e.g., a decreased blood oxygen level. A composition may be administered after (e.g., as soon as is practicable after) the onset of a disease or condition is detected or suspected. A composition may be administered after (e.g., as soon as is practicable after) a potential exposure to a coronavirus, for example, after a subject (e.g., the subject for immunization or the subject for treatment) has contact with an infected subject or learns they had contact with an infected subject that may be contagious.

Actual dosage levels of an agent of the disclosure (e.g., antibody or antigen-binding fragment thereof, or therapeutic agent) may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions employed herein, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

A composition described herein may be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

A dose may be based on the amount of the agent per kilogram of body weight of a subject (e.g., the subject for immunization or the subject for treatment). A dose of an agent (e.g., antibody) is in the range of 10-3000 mg/kg, e.g., 100-2000 mg/kg, e.g., 300-500 mg/kg/day for 1-10 or 1-5 days; e.g., 400 mg/kg/day for 3-6 days; e.g., 1 g/kg/d for 2-3 days In some embodiments, a dose may be based on the number of the enucleated cells per kilogram of body weight of a subject. In some embodiments, a dose may be is administered in a dosage amount of between about 1,000 cells/kg body weight to about 1,000,000,000,000 cells/kg body weight. In some embodiments, a dose may be is administered in a dosage amount of between about 1000 cells/kg body weight to about 1,000,000,000,000 cells/kg body weight. about 1,000 cells/kg body weight to about 10,000 cells/kg body weight, about 1,000 cells/kg body weight to about 100,000 cells/kg body weight, about 1,000 cells/kg body weight to about 1,000,000 cells/kg body weight, about 1,000 cells/kg body weight to about 10,000,000 cells/kg body weight, about 1,000 cells/kg body weight to about 100,000,000 cells/kg body weight, about 1,000 cells/kg body weight to about 1,000,000,000 cells/kg body weight, about 1,000 cells/kg body weight to about 10,000,000,000 cells/kg body weight, about 1,000 cells/kg body weight to about 100,000,000,000 cells/kg body weight, about 1,000 cells/kg body weight to about 1,000,000,000,000 cells/kg body weight, about 10,000 cells/kg body weight to about 100,000 cells/kg body weight, about 10,000 cells/kg body weight to about 1,000,000 cells/kg body weight, about 10,000 cells/kg body weight to about 10,000,000 cells/kg body weight, about 10,000 cells/kg body weight to about 100,000,000 cells/kg body weight, about 10,000 cells/kg body weight to about 1,000,000,000 cells/kg body weight, about 10,000 cells/kg body weight to about 10,000,000,000 cells/kg body weight, about 10,000 cells/kg body weight to about 100,000,000,000 cells/kg body weight, about 10,000 cells/kg body weight to about 1,000,000,000,000 cells/kg body weight, about 100,000 cells/kg body weight to about 1,000,000 cells/kg body weight, about 100,000 cells/kg body weight to about 10,000,000 cells/kg body weight, about 100,000 cells/kg body weight to about 100,000,000 cells/kg body weight, about 100,000 cells/kg body weight to about 1,000,000,000 cells/kg body weight, about 100,000 cells/kg body weight to about 10,000,000,000 cells/kg body weight, about 100,000 cells/kg body weight to about 100,000,000,000 cells/kg body weight, about 100,000 cells/kg body weight to about 1,000,000,000,000 cells/kg body weight, about 1,000,000 cells/kg body weight to about 10,000,000 cells/kg body weight, about 1,000,000 cells/kg body weight to about 100,000,000 cells/kg body weight, about 1,000,000 cells/kg body weight to about 1,000,000,000 cells/kg body weight, about 1,000,000 cells/kg body weight to about 10,000,000,000 cells/kg body weight, about 1,000,000 cells/kg body weight to about 100,000,000,000 cells/kg body weight, about 1,000,000 cells/kg body weight to about 1,000,000,000,000 cells/kg body weight, about 10,000,000 cells/kg body weight to about 100,000,000 cells/kg body weight, about 10,000,000 cells/kg body weight to about 1,000,000,000 cells/kg body weight, about 10,000,000 cells/kg body weight to about 10,000,000,000 cells/kg body weight, about 10,000,000 cells/kg body weight to about 100,000,000,000 cells/kg body weight, about 10,000,000 cells/kg body weight to about 1,000,000,000,000 cells/kg body weight, about 100,000,000 cells/kg body weight to about 1,000,000,000 cells/kg body weight, about 100,000,000 cells/kg body weight to about 10,000,000,000 cells/kg body weight, about 100,000,000 cells/kg body weight to about 100,000,000,000 cells/kg body weight, about 100,000,000 cells/kg body weight to about 1,000,000,000,000 cells/kg body weight, about 1,000,000,000 cells/kg body weight to about 10,000,000,000 cells/kg body weight, about 1,000,000,000 cells/kg body weight to about 100,000,000,000 cells/kg body weight, about 1,000,000,000 cells/kg body weight to about 1,000,000,000,000 cells/kg body weight, about 10,000,000,000 cells/kg body weight to about 100,000,000,000 cells/kg body weight, about 10,000,000,000 cells/kg body weight to about 1,000,000,000,000 cells/kg body weight, or about 100,000,000,000 cells/kg body weight to about 1,000,000,000,000 cells/kg body weight. In some embodiments, a dose may be is administered in a dosage amount of between about 1000 cells/kg body weight to about 1000000000000 cells/kg body weight. about 1,000 cells/kg body weight, about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, about 100,000,000,000 cells/kg body weight, or about 1,000,000,000,000 cells/kg body weight. In some embodiments, a dose may be is administered in a dosage amount of between about 1000 cells/kg body weight to about 1000000000000 cells/kg body weight. at least about 1,000 cells/kg body weight, about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, or about 100,000,000,000 cells/kg body weight. In some embodiments, a dose may be is administered in a dosage amount of between about 1000 cells/kg body weight to about 1000000000000 cells/kg body weight. at most about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, about 100,000,000,000 cells/kg body weight, or about 1,000,000,000,000 cells/kg body weight. In some embodiments, the cell without the nucleus is administered to the subject twice within at least an hour, 2 hours, 4 hours, 6 hours, 8 hours, 12 hours, 1 day, 2 days, a week, 2 weeks, 3 weeks, a month, 2 months, 3 months, 4 months, 5 months, 6 months, 7 months, 8 months, 9 months, 10 months, 11 months, a year, 2 years, 3 years, or 4 years.

Disclosed herein are methods of repeatedly administering the composition or the pharmaceutical composition to the subject in need thereof. In some embodiments, the first administrations of the composition or pharmaceutical composition comprising the enucleated cell normalizes blood vessel or lymphatic vessel. In some embodiments, the composition or pharmaceutical composition comprising the same enucleated cell can be subsequently administered to the subject for: maintaining the normalization of the blood vessel or lymphatic vessel; and delivering the exogenous agent for treating a disease or condition described herein.

### Kits

Disclosed herein, in some aspects, are kits for using the compositions described herein. In some embodiments, the kits disclosed herein may be used to treat a disease or condition in a subject. In some embodiments, the kits comprise an assemblage of materials or components apart from the composition. In some embodiments, the kit comprises nucleated cell described herein (e.g., nucleated cell engineered to express a targeting moiety (e.g., antibody or antigen-binding fragment thereof), a therapeutic agent, a transmembrane moiety, an immune-evading moiety, a heterologous gene produce, or a combination thereof. In some embodiments, the kit can include the population of cells as disclosed herein. In some embodiments, the kit can include enucleated cells obtained from the nucleated cells. In some embodiments, the kit can include a mixed population of nucleated cells and enucleated cells obtained from the nucleated cells. In some embodiments, the kit can include a substantially pure population of enucleated cells. In some embodiments, the kit comprises nucleated cells, enucleated cells, or a combination thereof suspended in at least one density gradient.

In some embodiments, the kit comprises a pharmaceutical formulation disclosed herein, comprising the enucleated cells engineered to express (and in some cases secrete) a targeting moiety (e.g., antibody or antigen-binding fragment thereof), a therapeutic agent, a transmembrane moiety, an immune-evading moiety, a heterologous gene produce, or a combination thereof. In some embodiments, the enucleated cell expresses or secretes the therapeutic agent such as an immune checkpoint molecule or an immune checkpoint inhibitor for treating a disease or condition in a subject. In some embodiments, the enucleated cells are further engineered to express a targeting moiety, such as a chemokine receptor, an integrin signaling molecule or an antibody or antigen-binding fragment thereof that enables the enucleated cells to efficiently migrate to the target tissue in a subject, once administered. In some embodiments, the kits further comprise an additional therapeutic agent, such as those disclosed herein. In some embodiments, the kit further comprises instructions for administering the pharmaceutical formulation and/or additional therapeutic agent to the subject to treat a disease or a condition in the subject such as cancer. In some embodiments, the cancer comprises cancer of the lung tissue. In some embodiments, the cancer is lung cancer.

In some embodiments, the kit comprises components for purifying enucleated cells from nucleated cells or other cellular debris. For example, the kit can include filter membranes with different pore sizes to isolate and purify the enucleated cells. In some embodiments, the kit comprises components for staining and selecting for enucleated cells. For example, the kit can include florescent dye for staining nucleus, where the nucleated cell can be stained and selectively removed, leaving a population of enucleated cells.

In some embodiments, the kit described herein comprises components for selecting for a homogenous population of the enucleated cells. In some embodiments, the kit described herein comprises components for selecting for a heterogenous population of the enucleated cells. In some embodiments, the kit comprises the components for assaying the number of units of a biomolecule (e.g., a therapeutic agent) synthesized, and/or released or expressed on the surface by the enucleated cell. In some embodiments, the kit comprises components for performing assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., cancer) in a subject. In some embodiments, the kit is configured particularly for the purpose of treating mammalian subjects. In some embodiments, the kit is configured particularly for the purpose of treating human subjects.

A container may be included in the kit. Instructions for use may be included in the kit. In some embodiments, the kit comprises instructions for administering the composition to a subject in need thereof. In some embodiments, the kit comprises instructions for further engineering the composition to express a biomolecule (e.g., a therapeutic agent). In some embodiments, the kit comprises instructions thawing or otherwise restoring biological activity of the composition, which may have been cryopreserved, lyophilized, or cryo-hibernated during storage or transportation. In some embodiments, the kit comprises instructions for measure viability of the restored compositions, to ensure efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components may be contained in suitable packaging material(s).

### Definitions

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, platforms, compositions, pharmaceutical compositions, and kits described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease may be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level.

The terms “individual” or “subject” are used interchangeably and encompass mammals. Non-limiting examples of mammal include any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The mammal may be a human. The term “animal” as used herein comprises human beings and non-human animals. In one embodiment, a “non-human animal” is a mammal, for example a rodent such as rat or a mouse. A “patient,” as used herein refers to a subject that has, or has been diagnosed with, a disease or a condition described herein.

The term “immune-evading moiety,” as used herein, refers to a signaling peptide, or portion thereof, that reduces cellular phagocytosis through its interaction with a signal receptor protein expressed by phagocytic cells, such as macrophages and dendritic cells. In some embodiments, the immune-evading moiety blocks immune cell recognition or immune cell activation.

The term “targeting moiety,” as used herein, refers to an entity that guides a cell, such as for e.g., an enucleated cell, to a target tissue or cell. The targeting moiety can be virtually any biomolecule, including a protein, polypeptide, a sugar, a nucleic acid, or a small molecule, or portions thereof.

The term “transmembrane moiety,” as used herein, refers to an entity that spans (at least partially) the cell membrane of a cell (e.g., enucleated cell).

The terms “expression” or “expressing” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state.

As used herein, a “cell” generally refers to a biological cell.

As used herein, “enucleation” is the rendering of a cell to a non-replicative state, such as, for example, through removal of the nucleus.

As used herein, the term “cytoplast,” “cell without a nucleus,” or “enucleated cell” are used interchangeably to refer to a nucleus-free cell that was obtained from a previously nucleated cell (e.g., any cell described herein). In some embodiments, the nucleated cell comprises cell organelles and the cytoplast derived from the nucleated cell retains such organelles, which in some cases, enables cellular functions such as cell motility, protein synthesis, protein secretion, and the like. In some embodiments, “obtaining” does not involve differentiating the nucleated cell into an enucleated cell using natural processes or otherwise.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. The term “gene” is to be interpreted broadly, and may encompass mRNA, cDNA, cRNA and genomic DNA forms of a gene.

In some uses, the term “gene” encompasses the transcribed sequences, including 5′ and 3′ untranslated regions (5′-UTR and 3′-UTR), exons and introns. In some genes, the transcribed region may contain “open reading frames” that encode polypeptides. In some uses of the term, a “gene” comprises only the coding sequences (e.g., an “open reading frame” or “coding region”) necessary for encoding a polypeptide. In some embodiments, genes do not encode a polypeptide, for example, ribosomal RNA genes (rRNA) and transfer RNA (tRNA) genes. In some embodiments, the term “gene” includes not only the transcribed sequences, but in addition, also includes non-transcribed regions including upstream and downstream regulatory regions, enhancers and promoters. The term “gene” may encompass mRNA, cDNA and genomic forms of a gene.

The term “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments.

As used herein, the term “package” refers to a suitable solid matrix or material such as glass, plastic, paper, foil, and the like, capable of holding the individual kit components. For example, a package may be a glass vial or prefilled syringes used to contain suitable quantities of the pharmaceutical. The packaging material has an external label which indicates the contents and/or purpose of the kit and its components.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure, and may perform any function, known or unknown. A polynucleotide comprises one or more analogs (e.g., altered backbone, sugar, or nucleobase). Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

As used herein, the terms “polypeptide,” “peptide” and “protein” may be used interchangeably herein in reference to a polymer of amino acid residues. A protein may refer to a full-length polypeptide as translated from a coding open reading frame, or as processed to its mature form, while a polypeptide or peptide may refer to a degradation fragment or a processing fragment of a protein that nonetheless uniquely or identifiably maps to a particular protein. A polypeptide may be a single linear polymer chain of amino acids bonded together by peptide bonds between the carboxyl and amino groups of adjacent amino acid residues. Polypeptides may be modified, for example, by the addition of carbohydrate, phosphorylation, etc.

As used herein, the terms “fragment,” or “portion,” or equivalent terms may refer to a portion of an entity that has less than the full length of the entity and optionally maintains the function of the entity. In some embodiments, the entity is a protein.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions, such as stringent conditions used in the relevant field) to hybridization with non-target sequences during a hybridization reaction. Typically, hybridizable sequences share a degree of sequence complementarity over all or a portion of their respective lengths, such as between 25%-100% complementarity, including greater than or equal to about 25%, 30%, 35%, 40%, 45%, 50%, 55%, 60%, 65%, 70%, 75%, 80%, 85%0, 90%, 91%, 92%, 93%, 94%, 95%, 96%, 97%, 98%, 99%, and 100% sequence complementarity. Sequence identity, such as for the purpose of assessing percent complementarity, may be measured by any suitable alignment algorithm, including but not limited to the Needleman-Wunsch algorithm (see e.g., the EMBOSS Needle aligner available at www.ebi.ac.uk/Tools/psa/emboss_needle/nucleotide.html, optionally with default settings), the BLAST algorithm. Optimal alignment may be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (e.g., gaps may be introduced in one or both of the candidate and reference sequences for optimal alignment and non-homologous sequences may be disregarded for comparison purposes). Alignment, for purposes of determining percent identity, may be achieved in various ways that are known in the relevant field. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “in vivo” may be used to describe an event that takes place in an organism, such as a subject's body.

As used herein, the term “ex vivo” may be used to describe an event that takes place outside of an organism, such as subject's body. An “ex vivo” assay cannot be performed on a subject. Rather, it may be performed upon a sample separate from a subject. Ex vivo may be used to describe an event occurring in an intact cell outside a subject's body.

As used herein, the term “in vitro” may be used to describe an event that takes places contained in a container for holding laboratory reagent such that it is separated from the living biological source organism from which the material is obtained. In vitro assays may encompass cell-based assays in which cells alive or dead are employed. In vitro assays may also encompass a cell-free assay in which no intact cells are employed.

“Treat, “treating,” or “treatment,” as used herein, refers to alleviating or abrogating a disorder, disease, or condition; or one or more of the symptoms associated with the disorder, disease, or condition; or alleviating or eradicating a cause of the disorder, disease, or condition itself. Desirable effects of treatment may include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “effective amount” and “therapeutically effective amount,” as used interchangeably herein, generally refer to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component may be “pharmaceutically acceptable” in the sense of being compatible with the other ingredients of a pharmaceutical formulation. It may also be suitable for use in contact with the tissue or organ of humans and animals without excessive toxicity, irritation, allergic response, immunogenicity, or other problems or complications, commensurate with a reasonable benefit/risk ratio.

As used herein, the term “administration,” “administering” and variants thereof means introducing a composition or agent into a subject and includes concurrent and sequential introduction of a composition or agent. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and administration by another. A suitable route of administration allows the composition or the agent to perform its intended function. For example, if a suitable route is intravenous, the composition is administered by introducing the composition or agent into a vein of the subject. Administration may be carried out by any suitable route. In some embodiments, the administering is intravenous administration. In some embodiments, the administering is pulmonary administration. In some embodiments, the administering is inhalation.

The term “pharmaceutical composition” refers to a mixture of a composition disclosed herein with other chemical components, such as diluents or carriers (e.g., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition may facilitate administration of the composition to an organism. Multiple techniques of administering a compound exist in the art including, but not limited to, oral, injection, aerosol, parenteral, and topical administration.

The term “fusogenic protein”, as used herein, refers to a polypeptide that, when expressed on the surface of cell, such as the enucleated cell, facilitates fusion of cell-to-cell membranes of the cell expressing the fusogenic protein and a target cell.

While preferred embodiments of the present inventive concepts have been shown and described herein, it will be obvious to those skilled in the art that such embodiments are provided by way of example only. It is not intended that the inventive concepts be limited by the specific examples provided within the specification. While the inventive concepts have been described with reference to the aforementioned specification, the descriptions and illustrations of the embodiments herein are not meant to be construed in a limiting sense. Numerous variations, changes, and substitutions will now occur to those skilled in the art without departing from the inventive concepts. Furthermore, it shall be understood that all aspects of the inventive concepts are not limited to the specific depictions, configurations or relative proportions set forth herein which depend upon a variety of conditions and variables. It should be understood that various alternatives to the embodiments of the inventive concepts described herein may be employed in practicing the inventive concepts. It is therefore contemplated that the inventive concepts shall also cover any such alternatives, modifications, variations or equivalents. It is intended that the following claims define the scope of the inventive concepts and that methods and structures within the scope of these claims and their equivalents be covered thereby.

### EMBODIMENTS

Embodiment 1. A method for obtaining a population of cells from a plurality of cells, comprising: contacting the plurality of cells with a polysaccharide density gradient generated by fixed angle centrifugation, wherein the population of cells accumulates at a density range in the polysaccharide density gradient.

Embodiment 2. A method for enucleating a population of cells from a plurality of cells, comprising: contacting the plurality of cells with a toxin, said toxin inhibits cytoskeletal formation of the plurality of cells; contacting the plurality of cells with a polysaccharide density gradient generated by fixed angle centrifugation, wherein the population of cells are enucleated by the fixed angle centrifugation and accumulates at a density range in the polysaccharide density gradient.

Embodiment 3. The method of Embodiment 1 or Embodiment 2, further comprising removing the population of cells from the polysaccharide density gradient.

Embodiment 4. The method of Embodiment 2, wherein the toxin comprises a mycotoxin.

Embodiment 5. The method of Embodiment 4, wherein the mycotoxin comprises a cytochalasin.

Embodiment 6. The method of Embodiment 5, wherein the cytochalasin comprises a cytochalasin B.

Embodiment 7. The method of Embodiment 2, wherein the population of cells, after enucleation, comprises a decreased density compared to the plurality of cells having nucleus.

Embodiment 8. The method of any one of Embodiments 1-7, wherein the polysaccharide density gradient, prior to the fixed angle centrifugation, comprises a plurality of polysaccharide solutions comprising different polysaccharide concentrations.

Embodiment 9. The method of any one of Embodiments 1-8, wherein the fixed angle centrifugation comprises centrifugating the plurality of cells in the polysaccharide density gradient at a 23-degree angle, at a 24-degree angle, at a 25-degree angle, at a 45-degree angle, at a 90-degree angle, or at a 180-degree angle.

Embodiment 10. The method of any one of Embodiments 1-9, wherein the fixed angle centrifugation comprises centrifugation at about 126000 RCF.

Embodiment 11. The method of any one of Embodiments 1-9, wherein the fixed angle centrifugation comprises centrifugation at an r average of about 85,000 RCF.

Embodiment 12. The method of any one of Embodiments 1-9, wherein the fixed angle centrifugation comprises centrifugation at an r max of about 127,000 RCF.

Embodiment 13. The method of any one of Embodiments 1-12, wherein the fixed angle centrifugation comprises acceleration comprising about 5.2 minutes to reach RCF for centrifugation.

Embodiment 14. The method of any one of Embodiments 1-13, wherein the fixed angle centrifugation comprises deceleration comprising about 4.0 minutes to reach a stoppage of the fixed angle centrifugation.

Embodiment 15. The method of any one of Embodiments 1-14, wherein the fixed angle centrifugation comprises centrifugation for about 60 minutes.

Embodiment 16. The method of any one of Embodiments 1-15, wherein the polysaccharide density gradient comprises a plurality of polysaccharide solutions.

Embodiment 17. The method of Embodiment 16, wherein the plurality of polysaccharide solutions comprises at least three polysaccharide solutions.

Embodiment 18. The method of Embodiment 17, wherein the plurality of polysaccharide solutions comprises: a 12.5% polysaccharide solution, a 15% polysaccharide solution, a 16% polysaccharide solution, a 17% polysaccharide solution, a 25% polysaccharide solution, or a combination thereof.

Embodiment 19. The method of any one of Embodiments 1-18, wherein the population of cells accumulates at the density range of about 15% polysaccharide.

Embodiment 20. The method of any one of Embodiments 1-19, wherein the polysaccharide density gradient comprises a Ficoll density gradient.

Embodiment 21. The method of any one of Embodiments 1-20, wherein the plurality of cells comprises a heterologous polynucleotide.

Embodiment 22. The method of any one of previous Embodiments, further comprising cryopreserving the population of cells.

Embodiment 23. The method of Embodiment 22, further comprising thawing the population of cells, wherein, following the thawing, an enucleated cell of the population of cells is as viable as an otherwise comparable enucleated cell that is not cryopreserved.

Embodiment 24. The method of any one of previous Embodiments, wherein the population of cells comprises stem cells.

Embodiment 25. The method of Embodiment 24, wherein the stem cells comprise induced pluripotent stem cells (iPSCs), adult stem cells, mesenchymal stromal cells, embryonic stem cells, fibroblasts, or immortalized cells from a cell line, or a combination thereof.

Embodiment 26. The method of Embodiment 25, wherein the stem cells comprise the mesenchymal stromal cells.

Embodiment 27. The method of any one of Embodiments 1-23, wherein the population of cells comprises immune cells.

Embodiment 28. The method of Embodiment 25, wherein the immune cells comprise lymphocytes or natural killer cells.

Embodiment 29. The method of any one of Embodiments 1-26, wherein the population of cells comprise one or more intracellular organelles for synthesis or secretion of an exogenous polypeptide in absence of a nucleus.

Embodiment 30. The method of Embodiment 29, wherein the exogenous polypeptide is encoded by the heterologous polynucleotide.

Embodiment 31. The method of Embodiment 29, wherein the exogenous polypeptide comprises a therapeutic agent.

Embodiment 32. The method of any one of Embodiments 1-31, wherein the population of cells comprise at least one targeting moiety.

Embodiment 33. The method of any one of Embodiments 1-32, wherein the population of cells comprise at least one fusogenic moiety.

Embodiment 34. The method of any one of Embodiments 1-33, wherein the population of cells comprise at least one immune evasion moiety.

Embodiment 35. The method of any one of Embodiments 1-34, wherein an enucleated cell of the population of cells has a diameter comprising less than or equal to about 10%, about 20%, about 30%, about 40%, about 50%, about 60%, about 70%, about 80%, about 90%, about 95%, or about 99% of an average diameter of the nucleated cells.

Embodiment 36. The method of any one of Embodiments 1-34, wherein an enucleated cell of the population of cells has a diameter comprising more than or equal to about 5 μm, about 10 μm, about 20 μm, about 30 μm, about 40 μm, about 50 μm, about 60 μm, about 70 μm, about 80 μm, or about 90 μm.

Embodiment 37. The method of Embodiment 36, wherein the diameter comprises about 8 μm.

Embodiment 38. A pharmaceutical composition comprising: the population of cells of any one of Embodiments 1-37; and a pharmaceutically acceptable: excipient, carrier, or diluent.

Embodiment 39. The pharmaceutical composition of Embodiment 38, wherein the pharmaceutical composition is in a unit dose form.

Embodiment 40. The pharmaceutical composition of Embodiment 38 or Embodiment 39, wherein the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof, to a subject.

Embodiment 41. The pharmaceutical composition of Embodiment 40, wherein the pharmaceutical composition is formulated for administering intravenously.

Embodiment 42. The pharmaceutical composition of any one of Embodiments 38-41, further comprising at least one additional active agent.

Embodiment 43. The pharmaceutical composition of Embodiment 42, wherein the at least one additional active agent comprises a cytokine, a growth factor, a hormone, an enzyme, a small molecule, a compound, or a combination thereof.

Embodiment 44. A kit comprising: the population of cells of any one of Embodiments 1-37 or the pharmaceutical composition of any one of Embodiments 38-43; and a container.

Embodiment 45. A method of treating a disease or condition of a subject comprising: administering to the subject a therapeutically effective amount of the population of cells of any one of Embodiments 1-37 or the pharmaceutical composition of any one of Embodiments 38-43.

Embodiment 46. A method of treating cancer in a subject, the method comprising: administering to the subject with cancer a therapeutically effective amount of the population of cells of any one of Embodiments 1-37 or the pharmaceutical composition of any one of Embodiments 38-43.

Embodiment 47. A method of treating a lung disease in a subject, the method comprising: administering to the subject with the lung disease a therapeutically effective amount of the population of cells of any one of Embodiments 1-37 or the pharmaceutical composition of any one of Embodiments 38-43.

### EXAMPLES

The following illustrative examples are representative of embodiments of the stimulation, systems, compositions, and methods described herein and are not meant to be limiting in any way.

### Example 1. Manufacturing of Enucleated Cells with Fixed Angle Centrifugation

### Material:

aMSCs hTERT CCP cell;
Ficoll PM400;
Ultrapure water (Invitrogen; 10977-015);
Refractometer (Reichert; 13940000);
10×MEM (Gibco; 11430-030);
7.5% Sodium Bicarbonate (Gibco; 25080-094);
100×Pen-Strep (Gibco; 15140-122);
0.22 μm membrane filter (Olympus; 25-227);
Alpha MEM (Gibco; 12561-056);
Premium FBS (Atlanta Biologics; S1150);
1M HEPES (Gibco; 15630-80);
100× Anti-Anti (Gibco; 15240-062);
100× GlutaMAX (Gibco; 35050-061);
Cytochalasin B (Cyto B) from Drechslera dematioidea;
94 ml ultra-clear tubes (Beckman);
PBS (Gibco; 14190-144);
40 μm cell strainer (Falcon; 352340);
Type 45Ti Rotor;
Beckman L8M ultracentrifuge;
Stemfull low cell adhesion tubes (Sbio; MS90150Z);
0.4% Trypan blue (Invitrogen; T10282); and
Hoechst 33342 (ThermoFisher; H3570) [10 mg/mL stock; 2 μg/mL final; 1:5,000 final]

### Protocol:

### Generate a Stock of 2×MEM

1. For 50 mL created, added 10 mL of 10×MEM, exactly 2.94 mL NaHCO₃, 1 mL 100× Pen-Strep, and 36 mL of ultrapure water to each 50 ml tube
2. Filtered through a 0.22 μm filter, then made 50 mL aliquots and stored at 4° C. for 1 month or until sedimentation.

### Prepare Cells for Enucleation

1. Seeded 12-14 roller bottles to be ready on the day of enucleation.

### Day 0—Prepare the Enucleation Working Solutions/Gradient

1. Added 100 μL of [10 mg/mL] Cyto B to 400 μL of DMSO to generate a [2 mg/mL] working solution (Store leftover at 4° C. for up to 2 weeks)
2. Added 10 μL of 2 mg/mL Cyto B for every 1 mL of 2×MEM [Final: 20 μg/mL Cyto B]1 tube: 500 μL of 2 mg/mL Cyto B to 50 mL of 2×MEM (Table 1)

TABLE 1
Enucleation working solution

| Number of buckets | 1 |
|---|---|
| Volume of 2X MEM needed [ml] | 50 |
| Volume of 50% Ficoll needed [ml] | 26.2 |
| Volume of 1X to prepare | 47 |

3. Added equal amount of 2×MEM+Cyto B and Ultrapure water to generate a 1×MEM+Cyto B working solution [Final: 10 μg/mL Cyto B]
1 tube: 23.5 mL of 2×MEM+Cyto B to 23.5 mL of Ultrapure water
4. Added equal amount of 2×MEM+Cyto B and 50% Ficoll to generate a 25% Ficoll working solution [Final: 10 μg/mL Cyto B] in a 50 mL conical
1 tube: 26.2 mL of 2×MEM+Cyto B to 26.2 mL of 50% Ficoll
5. Vortexed the 25% Ficoll for 1 min, then let rest to allow bubbles to dissipate
6. While the bubbles were dissipating, retrieved 15/50 mL conical tubes (17%, 16%, 15%, and 12.5%), then added 1×MEM+Cyto B as below (Table 2):

TABLE 2
Ficoll layer solutions for enucleation
1 Bucket

| Ficoll layer | 1x MEM (ml) |
|---|---|
| 12.5% | 20 |
| 15% | 2 |
| 16% | 1.8 |
| 17% | 4.8 |
| 25% | 0 |
| Total | 28.6 |

7. Used 300 μL of the 25% Ficoll to measure the refractive index (range: 1.3790-1.3810). Added 2×MEM+Cyto B or 50% Ficoll+cyto B to achieve the optimal refractive index
8. Added 25% Ficoll+Cyto B to the 15/50 mL conical tubes as below to generate the respective Ficoll layers (Table 3)

TABLE 3
Ficoll layer solutions for enucleation
1 Bucket

| Ficoll layer | 25% Ficoll Volume [ml] |
|---|---|
| 12.5% | 20 |
| 15% | 3 |
| 16% | 3.2 |
| 17% | 10.2 |
| 25% | 16 |
| Total | 52.4 |

9. Vortexed the Ficoll gradient tubes then incubate at 37° C.+5% CO₂ for 1 h with loosened caps
10. Using a 3 mL syringe with a 21 G 1.5″ needle, carefully added the Ficoll gradients to a 94 mL ultra-clear tube as below:
Breaking the vacuum in the syringe before collecting the 25% Ficoll layer, then while adding, tilting the tube at a 450 angle away, and pointing the bevel towards the tube wall ~1 cm above the surface of the liquid. Keeping the tube on the foam stand and not lifting it. Do not check the band intensity after each layer.
11. Marked the lines for each interface on the outside of the tubes for:
1^{st} layer: 14.25 mL of 25% Ficoll (using serological pipette)
2^{nd} layer: 14.25 mL of 17% Ficoll (6 mL with syringe, 8 mL with serological pipette and 250 μL with pipette)
3^{rd} layer: 3.56 mL of 16% Ficoll (3 mL with syringe and 560 μL with pipette)
4^{th} layer: 3.56 mL of 15% Ficoll (3 mL with syringe and 560 μL with pipette)
5^{th} layer: 14.25 mL of 12.5% Ficoll (6 mL with syringe, 8 ml with serological pipette and 250 μL with pipette)
12. Covered the Ficoll gradients with parafilm and incubated at 37° C. with 5% CO₂ overnight
13. Incubated the leftover 12.5% Ficoll and 1×MEM solutions at 37° C. with 5% CO₂ overnight with loosened caps
14. Placed the Type 45Ti Beckman buckets in the incubator overnight to pre-warm

### Day 1

1. Harvested MSCs (12 bottles), then resuspended the pellets in 30 mL of PBS before counting: number of cells: 273 million (M); viability %: 95%; average size: 20.1 μm
2. Pipetted 142M cells/tube into a fresh 15/50 mL conical tube then centrifuged at 200×g for 5 minutes
3. Used a P1000 to resuspend 142M cells in 1 mL of 12.5% Ficoll, before adding an additional 21.78 mL of 12.5% Ficoll. Resuspended the cells at a concentration of 20 million cells per 22.78 mL of 12.5% Ficoll
4. Passed the cell-Ficoll mixture through a 40 μm cell strainer into a clean 50 mL tube ensuring that the media stuck to the underside of the filter was also collected
5. Using a 3 mL syringe with a 21 gauge 1.5″ needle, loaded the 22.78 mL cell suspension to the top of the Ficoll gradient (3 ml with syringe and rest with serological pipette)
6. Using a 3 mL syringe with a 21 gauge 1.5″ needle, added 16.35 mL of 1×MEM to the top of the Ficoll gradient (6 ml with syringe and 10.35 ml with serological pipette)
7. Added 89 mL of PBS to a 94 mL ultra-clear tube as a balance as needed. When balancing, ensured that the tube caps were tightened (with torque wrench on 11 N*m) and the tubes were dry. The liquid should be as full as possible. Ensured that the balance tube was not dented or weak. Balancing tubes should be used 2 or 3 times before discarding.
8. Loaded the tubes into the paired rotor buckets (1&4, 2&5, 3&6), screwed on the lids, and weighed. Added 1×MEM or add/remove PBS to balance the buckets within +0.05 g
9. Loosened the bucket caps and incubated the buckets for 35 minutes at 37° C. with 5% CO₂
10. While the tubes/buckets were incubating, pre-warmed the ultracentrifuge by running the Type 45Ti Beckman rotor at 5,000 rpm for 35 minutes at 35° C. empty.
11. Once pre-warmed, added the tubes to the Type 45 rotor after tightening the bucket screws using a torque wrench (11 N*m)
12. Ran the centrifuge at 33,000 rpm for 1 hour at 31° C. with the Acceleration and Deceleration each set to 7
13. Prepared the hood by obtaining 15 mL Stemfull low cell adhesion tubes and serum free complete alpha MEM (containing HEPES, GlutaMAX, and anti-anti)
14. At the end of the run, released the vacuum, carried the buckets to the hood, and removed the Ficoll gradient tube from the bucket using clean tweezers
15. Using a serological pipette/P1000 collected the layers into Stemfull low cell adhesion tubes as below:
   Top of the gradient through the top half of the 12.5% layer [Top]
   Bottom half of 12.5% layer through the top half of the 17% layer [Middle]
   Bottom half of the 17% layer and the 25% layer [Bottom]
16. Diluted the collected layers in more than 4 volumes of serum-free complete media (typically up to 15 mL) then centrifuged at 200×g for 10 minutes
17. Aspirated the supernatants to 4 mL then filled the tube with serum-free complete media before centrifuging at 200×g for 5 min
18. Aspirated the supernatants then resuspended the pellets in an appropriate amount of serum-free complete media (5 mL for larger pellets, 1 mL for small pellets)
19. Using trypan blue exclusion (10 μL of cells+10 μL trypan blue), count the number of cells in each layer
20. Added about 20,000 cells from each layer to a 96-well plate normalizing the volume to 90 μL using serum free media
21. Added 10 μL of 1:500 diluted Hoechst [20 μg/mL] to the cells [2 μg/mL final]
22. Incubated the cells at 37° C. with 5% CO₂ for 10 minutes
23. Imaged the cells using a fluorescence microscope in order to determine the enucleation efficiency (% of cells without Hoechst staining)

The centrifugation was performed in a Type 45 Ti fixed angle rotor. Following centrifugation, the enucleated cells were located in the 15% Ficoll layer of the Ficoll gradient (FIG. 3A). The number of cells isolated from the 15% Ficoll layer was 1.82×10⁶ cells, resulting in a 90% yield of enucleated cells following enucleation. The enucleated cells were imaged subsequent to fixed angle centrifugation. The enucleated cells were imaged with bright-field microscopy to show the yield of cells following enucleation (FIG. 3B, center image). The cells were subsequently stained with 4′, 6-diamidino-2-phenylindole (DAPI), which stains the nucleus of cells. The fluorescent microscopy images of DAPI-stained enucleated cells showed that greater than 95% of the cells isolated from the 15% Ficoll layer following centrifugation did not have a nucleus (FIG. 3B, right image).

### Example 2. Comparison Between Fixed Angle Centrifugation and Swinging Bucket Centrifugation

An example of swinging bucket centrifugation enucleation is described in U.S. patent application Ser. No. 16/636,249, filed on Aug. 7, 2018, which is hereby incorporated by reference in its entirety. For fixed angle centrifugation enucleation, the cells were gown to 80-90% confluency based on the cell type. Then, Ficoll gradients were prepared from 50% Ficoll as shown in Table 4. The 50% Ficoll, frozen or cryopreserved, was thawed in a heated 25° C. water bath for at least 10 minutes until the 50% Ficoll was completely thawed.

TABLE 4
Ficoll gradient composition

| | Volume of | Volume of | Volume of | Volume of |
|---|---|---|---|---|
| Number | 50% Ficoll | 2X MEM | 1X MEM | 25% Ficoll |
| of tubes | [ml] | [ml] | [ml] | [ml] |
| 1 | 30 | 55 | 50 | 60 |
| 2 | 60 | 120 | 100 | 120 |
| 3 | 84 | 160 | 140 | 168 |
| 4 | 108 | 210 | 180 | 216 |
| 5 | 132 | 250 | 220 | 264 |
| 6 | 156 | 300 | 260 | 312 |

2×MEM from the 4° C. refrigerator was removed and placed in 25° C. water-bath for at least 15 minutes. Cytochalasin B aliquots (10 mg/mL) were also thawed at room temperature. 120 μl Cytochalasin B (10 mg/mL) was transferred into a fresh Eppendorf with 480 μl of DMSO. The Cytochalasin B solution was added to the 2×MEM for a final concentration of 20 μg/mL (100 μl of 2 mg/mL solution per 9.9 mL of 2×MEM).

To prepare 1×MEM, equal volumes of ultra-pure water and 2×MEM were combined. To prepare 25% Ficoll, equal volume of 2×MEM With Cytochalasin B and 50% Ficoll were mixed by vortex for 30 seconds at 3000 rpm. To prepare additional Ficoll gradient, the 2×MEM and 50% Ficoll were mixed to generate the gradients as shown in Table 4. The quantity of the Ficoll gradient was assessed by refraction index to determine the correction centration of the Ficoll gradient. The Ficoll gradient could then be overlaid with the cells for enucleation. Table 5 illustrates an example of the number of tubes (e.g., Ultraclear tube) and their respective Ficoll gradients subjected to the centrifugation. First layer of the Ficoll gradient loaded in the tube was 25% Ficoll at 14.25 mL. Second layer of the Ficoll gradient in the tube was 17% Ficoll at 14.25 mL. Third layer of the Ficoll gradient in the tube was 16% Ficoll at 3.56 ml. Fourth layer of the Ficoll gradient in the tube was 15% Ficoll at 3.56 mL. Fifth layer of the Ficoll gradient in the tube was 12.5% Ficoll at 14.25 mL.

TABLE 5
Ficoll gradient layer

| Number of tubes | 25% | 17% | 16% | 15% | 12.5% |
|---|---|---|---|---|---|
| 1 | 16 | 10.2 | 3.2 | 3 | 20 |
| 2 | 32 | 20.4 | 6.4 | 6 | 40 |
| 3 | 46 | 29.3 | 9.2 | 8.6 | 57.5 |
| 4 | 60 | 38.25 | 12 | 11.25 | 75 |
| 5 | 75 | 47.81 | 15 | 14.06 | 93.75 |
| 6 | 90 | 57.375 | 18 | 16.875 | 112.5 |

After the Ficoll layers were loaded, the top of each ultraclear tube was covered with parafilm. Each Ficoll layer was marked with a marker and incubated overnight at 37° C. in CO₂ incubator together with the 1×MEM, 12.5% Ficoll, any excess 25% Ficoll, and 2×MEM with loosened caps. Centrifuge rotor (e.g., Type 45Ti rotor and buckets) were placed in the 37° C. incubator without CO₂.

Cells (84-177×10⁶ MSC's per Ficoll Gradient Tube) were collected and resuspended with PBS to bring the concentration of the cells between 1-5×10⁶ ml for determining the number and viability of the cells with Trypan blue assay. About 84-177×10⁶ cells were loaded into a 50 mL conical tube. The cells were washed and resuspended with 12.5% Ficoll. The cells were then strained drop-wise through a strainer into a new 50 mL conical tube. The number and viability of the cells that passed through the straining was determined again with Trypan blue assay.

Using a 3.2 mL syringe with 21 gauge size needle, 3 mL of 12.5% Ficoll with cells was collected and overlaid on top of the Ficoll in the Ultraclear tube with the bevel against the side of the tube while the needle was about 1 cm above the liquid surface. Then, slowly release the 12.5% Ficoll with cells was released at a rate of 1 mL per 15 seconds. For each tube, a total of 6 ml of 12.5% Ficoll with cells was added). After loading the cells, 10.35 mL to a of 16.35 of 1×MEM was added to each tube to balance the weight of each tube to within +/−0.05 g for the centrifugation. The tubes containing the cells were then centrifuged in the ultracentrifuge for 33,000 rpm (or 85,000 RCF) for 60 minutes, acceleration to 7 and deceleration to 7.

Once the ultracentrifuge run was completed, each tube four layers: top of the 12.5% having some cells and a lot of cell debris; 12.5/15% interface having high number of enucleated cells; 17%—high or low number of enucleated cells; and bottom of 25% having nucleated cells and free nuclei. The layers having the enucleated cells were collected, washed, and pelleted in a conical tube. The enucleated cells were then resuspended for additional assay and for Hoechst assay to determine the enucleation efficiency. The enucleated cells were also cultured and allowed to attach to cell culture dish or plates for additional assays.

The enucleated cells were then either: directly assessed for their capability for secreting IL-12 to exert therapeutic efficacy; or first cryopreserved, thawed, and then assessed for the IL-12 secretion for exerting therapeutic efficacy. For in vitro IL-12 secretion assay, nucleated and enucleated MSCs (human Wharton's Jelly or human umbilical cord cells) were resuspended in antibiotic free media to 1×10⁶ cells/ml. Then, a total of 1 μg of mouse IL-12 mRNA was mixed together with MessengerMAX in OptiMEM according to manufacturer's protocol and incubated for five minutes at room temperature. The mRNA-MessengerMAX mixture was added to the cells and incubated for an additional 30 minutes in 37° C., 5% CO₂ incubator. Cells were washed once and seeded at 25,000 cells per well in a 24-well. The conditioned media was collected every 24 hours and analyzed.

Table 6 illustrates comparison of human adipose mesenchymal stem cells (MSCs) enucleation using two different rotors: swing bucket (SW41) and fixed angle (Type45Ti). The average data was collected from at least three runs. As shown in Table 6, the fixed angle centrifugation allowed more cells to be enucleated per run (852×10⁶ cells compared to 120×10⁶ cells for swinging bucket centrifugation). For both the fixed angle centrifugation and the swinging bucket centrifugation, the RCF (average) value was 85,000. For fixed angle centrifugation, the maximum RCF was 126,847. For swinging bucket centrifugation, the maximum RCF was 115,839. The fixed angle centrifugation also yielded increased percentage of enucleated cell per run (65% compared to 50% obtained from swinging bucket centrifugation). Both fixed angle centrifugation and swinging bucket centrifugation yielded similar enucleation efficiency.

TABLE 6
Comparison of human adipose tissue MSCs enucleation

| | SW41 | Type45Ti |
|---|---|---|
| Cell loaded per rotor [10\hat{ }6] | 120 | 852 |
| Centrifugation | 26,000 rpm | 33,000 rpm |
| | 60′ | 60′ |
| Average enucleation efficiency | 95% | 95% |
| Average enucleation yield | 50% | 65% |
| Average enucleated cell size | 15.5 | 15.3 |
| [μm] | | |

While the foregoing disclosure has been described in some detail for purposes of clarity and understanding, it will be clear to one skilled in the art from a reading of this disclosure that various changes in form and detail may be made without departing from the true scope of the disclosure. For example, all the techniques and apparatus described above may be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A method for obtaining a population of enucleated cells, the method comprising:
contacting a plurality of cells with a particle density gradient generated by fixed angle centrifugation to produce the population of enucleated cells,
wherein the population of enucleated cells accumulates at a density range in the particle density gradient.

2. The method of claim 1, further comprising:
contacting the plurality of cells with a toxin, wherein the toxin inhibits cytoskeletal formation of the plurality of cells.

3. The method of claim 1 or claim 2, further comprising removing the population of enucleated cells from the particle density gradient.

4. The method of claim 2, wherein the toxin comprises a mycotoxin.

5. The method of claim 4, wherein the mycotoxin comprises a cytochalasin.

6. The method of claim 5, wherein the cytochalasin comprises a cytochalasin B.

7. The method of claim 2, wherein the population of cells, after enucleation, comprises a decreased density compared to the plurality of cells having a nucleus.

8. The method of any one of claims 1-7, wherein the particle density gradient, prior to the fixed angle centrifugation, comprises a plurality of particle solutions comprising different particle concentrations.

9. The method of any one of claims 1-8, wherein the fixed angle centrifugation comprises centrifugating the plurality of cells in the particle density gradient at a 23-degree angle, at a 24-degree angle, at a 25-degree angle, at a 45-degree angle, at a 90-degree angle, or at a 180-degree angle.

10. The method of any one of claims 1-9, wherein the fixed angle centrifugation comprises centrifugation at about 80,000 RCF, at about 83,000 RCF, at about 85,000 RCF, at about 87,000 RCF, at about 90,000 RCF, at about 93,000 RCF, at about 95,000 RCF, at about 97,000 RCF, at about 100,000 RCF, at about 103,000 RCF, at about 105,000 RCF, at about 107,000 RCF, at about 110,000 RCF, at about 113,000 RCF, at about 115,000 RCF, at about 117,000 RCF, at about 120,000 RCF, at about 123,000 RCF, at about 125,000 RCF, at about 127,000 RCF, at about 130,000 RCF, at about 133,000 RCF, at about 135,000 RCF, or at about 137,000 RCF,

11. The method of claim 10, wherein the fixed angle centrifugation comprises centrifugation at about 85,000 RCF.

12. The method of claim 10, wherein the fixed angle centrifugation comprises centrifugation at about 127,000 RCF.

13. The method of any one of claims 1-12, wherein the fixed angle centrifugation comprises centrifugation at an r average of about 85,000 RCF.

14. The method of any one of claims 1-13, wherein the fixed angle centrifugation comprises centrifugation at an r max of about 127,000 RCF.

15. The method of any one of claims 1-14, wherein the fixed angle centrifugation comprises acceleration comprising about 5.2 min to reach RCF for centrifugation.

16. The method of any one of claims 1-15, wherein the fixed angle centrifugation comprises deceleration comprising about 4.0 min to reach a stoppage of the fixed angle centrifugation.

17. The method of any one of claims 1-16, wherein the fixed angle centrifugation comprises centrifugation for about 60 minutes.

18. The method of any one of claims 1-17, wherein the particle density gradient comprises a plurality of particle solutions.

19. The method of claim 18, wherein the plurality of particle solutions comprises at least three particle solutions.

20. The method of any one of claims 1-19, wherein the particle density gradient comprises a protein density gradient.

21. The method of claim 20, wherein the protein density gradient comprises a serum albumin gradient.

22. The method of any one of claims 1-19, wherein the particle density gradient comprises a polysaccharide density gradient.

23. The method of claim 22, wherein the polysaccharide density gradient comprises a plurality of polysaccharide solutions.

24. The method of claim 23, wherein the plurality of polysaccharide solutions comprises: a 12.5% polysaccharide solution, a 15% polysaccharide solution, a 16% polysaccharide solution, a 17% polysaccharide solution, a 25% polysaccharide solution, or any combination thereof.

25. The method of claim 24, wherein the population of enucleated cells accumulates at a density range of about 15% polysaccharide.

26. The method of claim 22, wherein the polysaccharide density gradient comprises a Ficoll density gradient.

27. The method of any one of claims 1-26, wherein the plurality of cells comprises a heterologous polynucleotide.

28. The method of any one of claims 1-27, further comprising cryopreserving the population of cells.

29. The method of any one of claims 1-28, wherein the fixed angle centrifugation comprises ultracentrifugation.

30. The method of any one of claims 1-29, wherein the plurality of cells comprises stem cells.

31. The method of claim 30, wherein the stem cells comprise induced pluripotent stem cells (iPSCs), adult stem cells, mesenchymal stromal cells, embryonic stem cells, fibroblasts, immortalized cells from a cell line, or any combination thereof.

32. The method of claim 31, wherein the stem cells comprise the mesenchymal stromal cells.

33. The method of any one of claims 1-32, wherein the plurality of cells comprises immune cells.

34. The method of claim 33, wherein the immune cells comprise lymphocytes or natural killer cells.

35. The method of any one of claims 1-34, wherein the plurality of cells comprises one or more intracellular organelles for synthesis or secretion of an exogenous polypeptide, wherein the one or more intracellular organelles is retained in the population of enucleated cells, and wherein the one or more intracellular organelles synthesizes or secretes the exogenous polypeptide in an absence of a nucleus.

36. The method of claim 35, wherein the plurality of cells comprises a heterologous polynucleotide encoding the exogenous polypeptide.

37. The method of claim 35, wherein the population of enucleated cells comprises a heterologous polynucleotide encoding the exogenous polypeptide, wherein the heterologous polynucleotide is introduced into the population of enucleated cells and the one or more intracellular organelles synthesizes or secretes the exogenous polypeptide in an absence of a nucleus.

38. The method of any one of claims 35-37, wherein the exogenous polypeptide comprises a therapeutic agent.

39. The method of any one of claims 1-38, wherein the population of enucleated cells comprise at least one targeting moiety.

40. The method of any one of claims 1-39, wherein the population of enucleated cells comprise at least one fusogenic moiety.

41. The method of any one of claims 1-40, wherein the population of enucleated cells comprise at least one immune evasion moiety.

42. The method of any one of claims 1-41, wherein an enucleated cell of the population of enucleated cells has a diameter comprising less than or equal to about 10%, about 20%, about 30%, about 40%, about 50%, about 60%, about 70%, about 80%, about 90%, about 95%, or about 99% of an average diameter of the nucleated cells.

43. The method of any one of claims 1-42, wherein an enucleated cell of the population of enucleated cells has a diameter comprising more than or equal to about 5 μm, about 10 μm, about 20 μm, about 30 μm, about 40 μm, about 50 μm, about 60 μm, about 70 μm, about 80 μm, or about 90 μm.

44. The method of claim 43, wherein the diameter comprises about 8 μm.

45. A pharmaceutical composition comprising:
(a) the population of enucleated cells produced by a method of any one of claims 1-40; and
(b) a pharmaceutically acceptable: excipient, carrier, or diluent.

46. The pharmaceutical composition of claim 45, wherein the pharmaceutical composition is in a unit dose form.

47. The pharmaceutical composition of claim 45 or 46, wherein the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or any combination thereof, to a subject.

48. The pharmaceutical composition of claim 47, wherein the pharmaceutical composition is formulated for administering intravenously.

49. The pharmaceutical composition of any one of claims 45-48, further comprising at least one additional active agent.

50. The pharmaceutical composition of claim 49, wherein the at least one additional active agent comprises a cytokine, a growth factor, a hormone, an enzyme, a small molecule, a compound, or any combination thereof.

51. A kit comprising:
a) the population of cells produced by a method of any one of claims 1-44 or the pharmaceutical composition of any one of claims 45-50; and
b) a container.

52. A kit comprising:
a) instructions for enucleating the plurality of cells according to a method of any one of claims 1-44 to produce the population of enucleated cells; and
b) one or more components of the polysaccharide density gradient.

53. A method of treating a disease or condition of a subject comprising: administering to the subject a therapeutically effective amount of the population of enucleated cells produced by a method of any one of claims 1-44 or the pharmaceutical composition of any one of claims 45-50.

54. A method of treating cancer in a subject, the method comprising: administering to the subject with cancer a therapeutically effective amount of the population of enucleated cells produced by a method of any one of claims 1-44 or the pharmaceutical composition of any one of claims 45-50.

55. A method of treating a lung disease in a subject, the method comprising: administering to the subject with the lung disease a therapeutically effective amount of the population of enucleated cells produced by a method of any one of claims 1-44 or the pharmaceutical composition of any one of claims 45-50.
